(19) United States
(12) Patent Application Publication
(10) Pub. No.: US 2019/0031624 A1
(43) Pub. Date: Jan. 31, 2019

(54) THERAPEUTIC COMPOUNDS AND METHODS TO TREAT INFECTION

(71) Applicants: RUTGERS, THE STATE UNIVERSITY OF NEW JERSEY, New Brunswick, NJ (US); TAXIS PHARMACEUTICALS, INC., Monmouth Junction, NJ (US)

(72) Inventors: Edmond J. LaVoie, New Brunswick, NJ (US); Ajit K. Parhi, New Brunswick, NJ (US); Yangsheng Sun, Monmouth Junction, NJ (US)

(21) Appl. No.: 16/046,696

(22) Filed: Jul. 26, 2018

Related U.S. Application Data
(60) Provisional application No. 62/538,547, filed on Jul. 28, 2017.

(51) Int. Cl. C07D 251/08; C07C 335/32; C07D 213/70; C07D 209/08; C07D 215/12; C07D 307/79; C07D 333/54; A61P 31/04

(52) U.S. Cl. CPC C07D 251/08; C07C 335/32; C07D 213/70; A61P 31/04; C07D 215/12; C07D 307/79; C07D 333/54; C07D 209/08

(57) ABSTRACT

Disclosed herein are compounds of formula I:

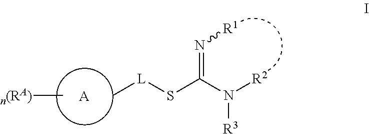

or a salt thereof and compositions comprising a compound of formula I or a pharmaceutically acceptable salt thereof. Also disclosed herein are methods for treating or preventing a bacterial infection in an animal comprising administering to the animal a compound of formula I or a pharmaceutically acceptable salt thereof, alone or in combination with a bacterial efflux pump inhibitor.

### PRIORITY APPLICATION

This application claims priority to U.S. Provisional Application No. 62/538,547 that was filed on Jul. 28, 2017. The entire content of the application referenced above is hereby incorporated by reference herein.

### BACKGROUND OF THE INVENTION

Antibiotics have been effective tools in the treatment of infectious diseases. However, bacteria have developed several different mechanisms to overcome the action of antibiotics. The emergence of Multidrug Resistant (MDR) bacterial pathogens has increased concerns as to the adequacy of current antimicrobials and pathogen treatment methods. The lethality of pathogens, such as Pseudomonas aeruginosa, has often led to treatment methods that are experimental or would otherwise normally be avoided in standard clinical practice. The growing threat from MDR pathogens highlights a critical need for additional antimicrobials. Thus, there is a pressing need for new antibiotics that exhibit novel mechanisms of action or circumvent conventional mechanisms of resistance.

Mechanisms of resistance can be specific such as for a molecule or a family of antibiotics, or the mechanisms can be non-specific. Several mechanisms of resistance can exist in a single bacterial strain, and those mechanisms may act independently or they may act synergistically to overcome the action of an antibiotic or a combination of antibiotics. Specific mechanisms include, for example, degradation of the drug, inactivation of the drug by enzymatic modification, and alteration of the drug target. Additional mechanisms of drug resistance include mechanisms in which access of the antibiotic to the target is prevented or reduced by decreasing the transport of the antibiotic into the cell or by increasing the efflux of the drug from the cell to the outside medium. Both of these mechanisms can lower the concentration of drug at the target site and allow bacterial survival in the presence of one or more antibiotics that would otherwise inhibit or kill the bacterial cells. Some bacteria utilize both mechanisms, combining low permeability of the cell wall (including membranes) with an active efflux of antibiotics. It has been shown that efflux of antibiotics can be mediated by more than one pump in a single organism and that almost all antibiotics are subject to resistance by this mechanism. For example, Pseudomonas aeruginosa expresses numerous efflux pumps including MexAB-OprM, MexCD-OprJ, MexEF-OprN, and MexXY-OprA(OprM) which actively efflux various antibacterial agents.

These multiple resistance mechanisms have become widespread and threaten the clinical utility of antibacterial therapy. The increase in antibiotic resistant strains has been particularly noted in major hospitals and care centers. The consequences of the increase in resistant strains include, for example higher morbidity and mortality, longer patient hospitalization, and an increase in treatment costs. Accordingly, there is a need for novel antibiotics and agents that can be used in combination to inhibit one or more of these mechanisms of bacterial resistance.

MreB is a well conserved and essential cytoskeleton-like protein, which represents a bacterial homolog of actin. Studies with various MreB homologs have established that this protein forms dynamic, actin-like helical filaments in an ATP- or GTP-dependent fashion. The filaments are localized within the bacterial cell on the inner surface of the cytoplasmic membrane. MreB protein provides a critical role in the maintenance of cell shape, polar protein localization, and/or chromosome segregation. Because of its essential role in bacterial growth and function, MreB inhibitors represent a novel class of antibacterial agents.

The MreB homolog of P. aeruginosa is essential for cell viability as well as maintenance of rod-like cell morphology. A22 (S-(3,4-Dichlorobenzyl)isothiourea) was identified as an effective MreB inhibitor in P. aeruginosa while screening libraries of compounds against a P. aeruginosa strains with defective efflux transporters.

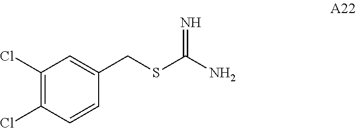 [SMILES: N=C(N)SCc1ccc(Cl)c(Cl)c1]

A22 is a substrate for the MexAB-OprM efflux transporter present in wild-type P. aeruginosa, which limited its efficacy and clinical promise. Thus, there is an ongoing need for antibacterial agents with novel mechanisms of action (e.g., MreB inhibitors). There is also a need for antibacterial agents (e.g., MreB inhibitors) that have improved properties such as improved formulation characteristics or lowered toxicity.

### SUMMARY OF THE INVENTION

Compounds disclose herein, when tested alone or in combination with a bacterial efflux pump inhibitor, exhibit antibacterial activities.

Accordingly, one embodiment provides a method of treating or preventing a bacterial infection in an animal (e.g., a mammal such as a human) comprising administering to the animal a bacterial efflux pump inhibitor and a compound of formula I:

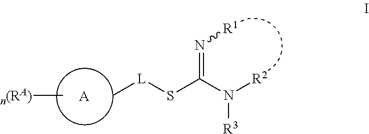

or a pharmaceutically acceptable salt thereof, wherein:

R¹ is hydrogen, (C₁-C₄)alkyl, or (C₁-C₄)haloalkyl, and R² is hydrogen, (C₁-C₄)alkyl, or (C₁-C₄)haloalkyl; or R¹ and R² taken together with the atoms to which they are attached form a tetrahydro-1,3,5-triazinyl which is optionally substituted with one or more R⁴ groups;

R³ is hydrogen, (C₁-C₄)alkyl or (C₁-C₄)haloalkyl;

each R⁴ is independently hydrogen, halo, hydroxyl, nitro, cyano, (C₁-C₆)alkyl, aryl or heteroaryl, wherein the (C₁-C₆)alkyl is optionally substituted with one or more groups selected from halo, hydroxy, nitro, cyano, (C₁-C₄)alkoxy, —NR^{X}R^{Y}, aryl, heteroaryl, aryloxy, or heteroaryloxy, wherein any ary, heteroaryl, aryloxy and heteroaryloxy is optionally substituted with one or more groups selected from halo, hydroxy, (C₁-C₄)alkyl, (C₁-C₄)haloalkyl, (C₁-C₄)alkoxy, nitro, cyano, or —NR^{X}R^{Y};

L is (C₁-C₅)alkylene that is optionally substituted with one or more R^{L} groups;

each R^{L} is independently selected from hydrogen, halo, hydroxy, nitro, cyano, or (C₁-C₄)alkoxy; or any two R^{L} groups that are attached to the same carbon taken together form a (C₃-C₆)carbocycle;

A is aryl or heteroaryl;

each R^{A} is independently selected from the group consisting of halo, cyano, nitro, (C₁-C₆)alkyl, (C₁-C₆)alkoxy, —NR^{X}R^{Y}, aryl or heteroaryl; wherein the (C₁-C₆)alkyl and (C₁-C₆)alkoxy is optionally substituted with one or more groups selected from oxo, halo, hydroxy, (C₁-C₄)alkoxy, nitro, cyano, or —NR^{X}R^{Y}; wherein the aryl and heteroaryl is optionally substituted with one or more groups selected from halo, hydroxy, (C₁-C₄)alkyl, (C₁-C₄)haloalkyl, (C₁-C₄)alkoxy, nitro, cyano, or —NR^{X}R^{Y};

each R^{X} and R^{Y} are independently hydrogen or (C₁-C₄)alkyl; or R^{X} and R^{Y} taken together with the nitrogen to which they are attached form pyrrolidinyl, piperidinyl, piperazinyl, or morpholinyl; and

n is 0, 1, 2, 3, or 4.

One embodiment provides a method of treating or preventing a bacterial infection in an animal (e.g., a mammal such as a human) comprising administering to the animal in need thereof a bacterial efflux pump inhibitor and a compound of formula I, or a pharmaceutically acceptable salt thereof, as described herein.

One embodiment provides a method of treating or preventing a bacterial infection in an animal (e.g., a mammal such as a human) comprising administering to the animal a compound of formula I, or a pharmaceutically acceptable salt thereof, as described herein.

One embodiment provides a method of treating or preventing a bacterial infection in an animal (e.g., a mammal such as a human) comprising administering to the animal in need thereof a compound of formula I, or a pharmaceutically acceptable salt thereof, as described herein.

One embodiment provides a method of treating or preventing a bacterial infection in an animal (e.g., a mammal such as a human) infected with bacteria comprising administering to the animal a bacterial efflux pump inhibitor and a compound of formula I, or a pharmaceutically acceptable salt thereof, as described herein and one or more antibacterial agents.

One embodiment provides a compound of formula Ia or Ib;

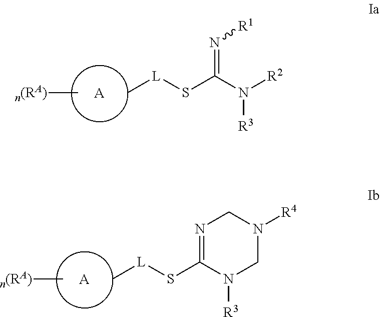

or a salt thereof, wherein:

R¹ is hydrogen, (C₁-C₄)alkyl, or (C₁-C₄)haloalkyl;

R² is hydrogen, (C₁-C₄)alkyl, or (C₁-C₄)haloalkyl;

R³ is hydrogen, (C₁-C₄)alkyl, or (C₁-C₄)haloalkyl;

R⁴ is hydrogen, (C₁-C₆)alkyl, aryl or heteroaryl, wherein the (C₁-C₆)alkyl is optionally substituted with one or more groups selected from halo, hydroxy, nitro, cyano, (C₁-C₄)alkoxy, —NR^{X}R^{Y}, aryl, heteroaryl, aryloxy, or heteroaryloxy, wherein any ary, heteroaryl, aryloxy and heteroaryloxy is optionally substituted with one or more groups selected from halo, hydroxy, (C₁-C₄)alkyl, (C₁-C₄)haloalkyl, (C₁-C₄)alkoxy, nitro, cyano, or —NR^{X}R^{Y};

L is (C₁-C₅)alkylene that is optionally substituted with one or more R^{L} groups;

each R^{L} is independently selected from hydrogen, halo, hydroxy, nitro, cyano, or (C₁-C₄)alkoxy; or any two R^{L} groups that are attached to the same carbon taken together form a (C₃-C₆)carbocycle;

A is aryl or heteroaryl;

each R^{A} is independently selected from the group consisting of halo, cyano, nitro, (C₁-C₆)alkyl, (C₁-C₆)alkoxy, —NR^{X}R^{Y}, aryl or heteroaryl; wherein the (C₁-C₆)alkyl and (C₁-C₆)alkoxy is optionally substituted with one or more groups selected from oxo, halo, hydroxy, (C₁-C₄)alkoxy, nitro, cyano, or —NR^{X}R^{Y}; wherein the aryl and heteroaryl is optionally substituted with one or more groups selected from halo, hydroxy, (C₁-C₄)alkyl, (C₁-C₄)haloalkyl, (C₁-C₄)alkoxy, nitro, cyano, or —NR^{X}R^{Y};

each R^{X} and R^{Y} are independently hydrogen or (C₁-C₄)alkyl; or R^{X} and R^{Y} taken together with the nitrogen to which they are attached form pyrrolidinyl, piperidinyl, piperazinyl, or morpholinyl; and

n is 0, 1, 2, 3, or 4;

provided that when the compound is a compound of formula Ia and A is phenyl, then A is substituted with at least one phenyl group; and

provided that the compound of formula Ib is not

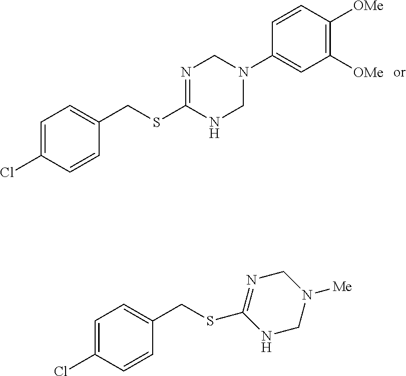 [SMILES: COc1ccc(N2CN=C(SCc3ccc(Cl)cc3)NC2)cc1OC ; CN1CN=C(SCc2ccc(Cl)cc2)NC1]

or a salt thereof.

One embodiment provides a pharmaceutical composition comprising a compound of formula Ia or Ib, or a pharmaceutically acceptable salt thereof as described herein, and a pharmaceutically acceptable vehicle.

One embodiment provides pharmaceutical composition comprising a compound of formula I, or a pharmaceutically acceptable salt thereof, as described herein, a bacterial efflux pump inhibitor and a pharmaceutically acceptable vehicle.

One embodiment provides a method of treating or preventing a bacterial infection in an animal (e.g., a mammal such as a human) comprising administering to the animal a compound of formula Ia or Ib, or a pharmaceutically acceptable salt thereof, as described herein.

One embodiment provides a method of treating or preventing a bacterial infection in an animal (e.g., a mammal such as a human) comprising administering to the animal a compound of formula Ia or Ib, or a pharmaceutically acceptable salt thereof, as described herein and a bacterial efflux pump inhibitor.

One embodiment provides a method of treating or preventing a bacterial infection in an animal (e.g., a mammal such as a human) comprising administering to the animal in need thereof a compound of formula Ia or Ib, or a pharmaceutically acceptable salt thereof, as described herein and a bacterial efflux pump inhibitor.

One embodiment provides a method of inhibiting bacterial protein MreB in a cell in vitro or in vivo comprising contacting the cell with a compound of formula Ia or Ib, or a pharmaceutically acceptable salt thereof, as described herein.

One embodiment provides a compound of formula I, or a pharmaceutically acceptable salt thereof, as described herein, combined with a bacterial efflux pump inhibitor, for use in medical therapy.

One embodiment provides a compound of formula I, or a pharmaceutically acceptable salt thereof, as described herein, combined with a bacterial efflux pump inhibitor, for the prophylactic or therapeutic treatment of a bacterial infection.

One embodiment provides the use of a compound of formula I, or a pharmaceutically acceptable salt thereof, as described herein, combined with a bacterial efflux pump inhibitor, for the preparation of a medicament for treating a bacterial infection in an animal (e.g., a mammal such as a human).

One embodiment provides a compound of formula Ia or Ib, or a pharmaceutically acceptable salt thereof, as described herein, for the prophylactic or therapeutic treatment of a bacterial infection.

One embodiment provides the use of a compound of formula Ia or Ib, or a pharmaceutically acceptable salt thereof, as described herein, for the preparation of a medicament for treating a bacterial infection in an animal (e.g., a mammal such as a human).

One embodiment provides processes and intermediates disclosed herein that are useful for preparing compounds of formula I, or salts thereof.

### DETAILED DESCRIPTION

The following definitions are used, unless otherwise described: halo or halogen is fluoro, chloro, bromo, or iodo. Alkyl and alkoxy, etc. denote both straight and branched groups but reference to an individual radical such as propyl embraces only the straight chain radical (a branched chain isomer such as isopropyl being specifically referred to).

As used herein, the term “(Cₐ-C_{b})alkyl” wherein a and b are integers refers to a straight or branched chain alkyl radical having from a to b carbon atoms. Thus when a is 1 and b is 6, for example, the term includes methyl, ethyl, n-propyl, isopropyl, n-butyl, isobutyl, sec-butyl, t-butyl, n-pentyl and n-hexyl.

The term “alkylene” by itself or as part of another substituent means a divalent radical derived from an alkane (including straight and branched alkanes), as exemplified by —CH₂—, —CH(CH₃)—, —C(CH₃)₂—, —CH[CH(CH₃)₂]—, —CH(CH₂CH₃)—, —CH₂CH₂CH₂CH₂— and —CH(CH₃)CH₂CH₂—.

The term “aryl” as used herein refers to a single aromatic ring or a multiple condensed ring system wherein the ring atoms are carbon. For example, an aryl group can have 6 to 10 carbon atoms, or 6 to 12 carbon atoms. Aryl includes a phenyl radical. Aryl also includes multiple condensed ring systems (e.g., ring systems comprising 2 rings) having about 9 to 12 carbon atoms or 9 to 10 carbon atoms in which at least one ring is aromatic. Such multiple condensed ring systems may be optionally substituted with one or more (e.g., 1 or 2) oxo groups on any cycloalkyl portion of the multiple condensed ring system. It is to be understood that the point of attachment of a multiple condensed ring system, as defined above, can be at any position of the ring system including an aryl or a cycloalkyl portion of the ring. Typical aryl groups include, but are not limited to, phenyl, indenyl, naphthyl, 1, 2, 3, 4-tetrahydronaphthyl, anthracenyl, and the like.

The term “heteroaryl” as used herein refers to a single aromatic ring or a multiple condensed ring system. The term includes single aromatic rings of from about 1 to 6 carbon atoms and about 1-4 heteroatoms selected from the group consisting of oxygen, nitrogen and sulfur in the rings. The sulfur and nitrogen atoms may also be present in an oxidized form provided the ring is aromatic. Such rings include but are not limited to pyridyl, pyrimidinyl, oxazolyl or furyl. The term also includes multiple condensed ring systems (e.g. ring systems comprising 2 rings) wherein a heteroaryl group, as defined above, can be condensed with one or more heteroaryls (e.g., naphthyridinyl), heterocycles, (e.g., 1, 2, 3, 4-tetrahydronaphthyridinyl), cycloalkyls (e.g., 5,6,7,8-tetrahydroquinolyl) or aryls (e.g. indazolyl) to form a multiple condensed ring system. Such multiple condensed ring systems may be optionally substituted with one or more (e.g., 1 or 2) oxo groups on the cycloalkyl or heterocycle portions of the condensed ring. In one embodiment a monocyclic or bicyclic heteroaryl has 5 to 10 ring atoms comprising 1 to 9 carbon atoms and 1 to 4 heteroatoms. It is to be understood that the point of attachment of a multiple condensed ring system (as defined above for a heteroaryl) can be at any position of the multiple condensed ring system including a heteroaryl, heterocycle, aryl or cycloalkyl portion of the multiple condensed ring system and at any suitable atom of the multiple condensed ring system including a carbon atom and heteroatom (e.g., a nitrogen). Exemplary heteroaryls include but are not limited to pyridyl, pyrrolyl, pyrazinyl, pyrimidinyl, pyridazinyl, pyrazolyl, thienyl, indolyl, imidazolyl, oxazolyl, thiazolyl, furyl, oxadiazolyl, thiadiazolyl, quinolyl, isoquinolyl, benzothiazolyl, benzoxazolyl, indazolyl, quinoxalyl, quinazolyl, 5,6,7,8-tetrahydroisoquinolinyl, benzofuranyl, benzimidazolyl and thianaphthenyl.

The term cycloalkyl, carbocycle, or carbocyclyl includes saturated and partially unsaturated carbocyclic ring systems. In one embodiment the cycloalkyl is a monocyclic carbocyclic ring. Such cycloalkyls include “(C₃-C₇)carbocyclyl” and “(C₃-C₅)cycloalkyl”.

The term “heterocyclyl” or “heterocycle” as used herein refers to a single saturated or partially unsaturated ring or a multiple condensed ring system. The term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen and sulfur in the ring. The ring may be substituted with one or more (e.g., 1, 2 or 3) oxo groups and the sulfur and nitrogen atoms may also be present in their oxidized forms. Such rings include but are not limited to azetidinyl, tetrahydrofuranyl or piperidinyl. It is to be understood that the point of attachment for a heterocycle can be at any suitable atom of the heterocycle Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl and tetrahydrothiopyranyl.

The term “haloalkyl” includes an alkyl group as defined herein that is substituted with one or more (e.g., 1, 2, 3, or 4) halo groups. One specific halo alkyl is a “(C₁-C₆)haloalkyl”.

The term “alkoxy” refers to —O(alkyl) and the term “haloalkoxy” refers to an alkoxy that is substituted with one or more (e.g., 1, 2, 3, or 4) halo.

The term “aryloxy” refers to —O-aryl and the term “heteroaryloxy” refers to —O— heteroaryl.

Specific values listed below for radicals, substituents, and ranges, are for illustration only; they do not exclude other defined values or other values within defined ranges for the radicals and substituents.

Specifically, (C₁-C₆)alkyl can be methyl, ethyl, propyl, isopropyl, butyl, iso-butyl, sec-butyl, pentyl, 3-pentyl, or hexyl; (C₁-C₆)alkoxy can be methoxy, ethoxy, propoxy, isopropoxy, butoxy, iso-butoxy, sec-butoxy, pentoxy, 3-pentoxy, or hexyloxy; (C₃-C₅)cycloalkyl can be cyclopropyl, cyclobutyl, cyclopentyl, or cyclohexyl; and heteroaryl can be furyl, imidazolyl, triazolyl, triazinyl, oxazolyl, isoxazolyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide).

### Bacterial Efflux Pump Inhibitor

An efflux pump inhibitor is a compound that interferes with the ability of an efflux pump to export a substrate. The inhibitor may have intrinsic antibacterial properties of its own.

In one embodiment, the bacterial efflux pump inhibitor is a compound of formula II:

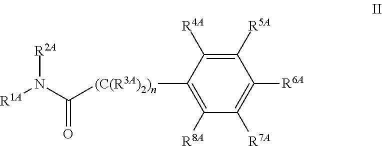

or a pharmaceutically acceptable salt thereof, wherein:

R^{1A} is (C₃-C₈)alkyl substituted with two or more groups selected from —NR^{b1}R^{c1}, —NHNH₂, —C(═NR^{a1})(NR^{b1}R^{c1}), —NR^{a1}C(═NR^{a1})(R^{d1}) and —NR^{a1}C(═NR^{a1})(NR^{b1}R^{c1});

R^{2A} is hydrogen or (C₁-C₃)alkyl;

each R^{3A} is independently hydrogen, halo or (C₁-C₄)alkyl;

R^{4A} is hydrogen, halo, (C₁-C₆)alkyl, (C₁-C₆)haloalkyl, (C₁-C₆)alkoxy, (C₁-C₄)haloalkoxy, aryl or heteroaryl wherein the aryl or heteroaryl is optionally substituted with one or more groups independently selected from halo, (C₁-C₄)alkyl, (C₁-C₄)haloalkyl, (C₁-C₄)alkoxy and (C₁-C₄)haloalkoxy;

R^{5A} is hydrogen, halo, (C₁-C₆)alkyl, (C₁-C₆)haloalkyl, (C₁-C₆)alkoxy, (C₁-C₄)haloalkoxy, aryl or heteroaryl wherein the aryl or heteroaryl is optionally substituted with one or more groups independently selected from halo, (C₁-C₄)alkyl, (C₁-C₄)haloalkyl, (C₁-C₄)alkoxy and (C₁-C₄)haloalkoxy;

R^{6A} is hydrogen, halo, (C₁-C₆)alkyl, (C₁-C₆)haloalkyl, (C₁-C₆)alkoxy, (C₁-C₄)haloalkoxy, aryl or heteroaryl wherein the aryl or heteroaryl is optionally substituted with one or more groups independently selected from halo, (C₁-C₄)alkyl, (C₁-C₄)haloalkyl, (C₁-C₄)alkoxy and (C₁-C₄)haloalkoxy;

R^{7A} is hydrogen, halo, (C₁-C₆)alkyl, (C₁-C₆)haloalkyl, (C₁-C₆)alkoxy, (C₁-C₄)haloalkoxy, aryl or heteroaryl wherein the aryl or heteroaryl is optionally substituted with one or more groups independently selected from halo, (C₁-C₄)alkyl, (C₁-C₄)haloalkyl, (C₁-C₄)alkoxy and (C₁-C₄)haloalkoxy;

R^{8A} is hydrogen, halo, (C₁-C₆)alkyl, (C₁-C₆)haloalkyl, (C₁-C₆)alkoxy, (C₁-C₄)haloalkoxy, aryl or heteroaryl wherein the aryl or heteroaryl is optionally substituted with one or more groups independently selected from halo, (C₁-C₄)alkyl, (C₁-C₄)haloalkyl, (C₁-C₄)alkoxy and (C₁-C₄)haloalkoxy;

each R^{a1} is independently hydrogen or (C₁-C₄)alkyl;

each R^{b1} and R^{C1} is independently hydrogen or (C₁-C₄)alkyl;

R^{d1} is (C₁-C₃)alkyl and

n is 0 or 1.

It is to be understood that the embodiments provided below are for compounds of formula II and all sub-formulas thereof (e.g., formulas IIa, IIb, IIc, IId, IIe, IIf, IIg). It is to be understood the two or more embodiments may be combined.

In one embodiment R^{2A} is hydrogen.

In one embodiment R^{3A} is hydrogen.

One embodiment provides a bacterial efflux pump inhibitor which is a compound of formula IIa:

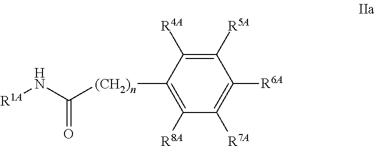

or a pharmaceutically acceptable salt thereof.

In one embodiment R^{8A} is hydrogen.

In one embodiment R^{4A} is hydrogen, (C₁-C₆)haloalkyl or aryl wherein the aryl is optionally substituted with one or more (e.g., 1, 2, 3, 4 or 5) groups independently selected from halo, (C₁-C₄)alkyl, (C₁-C₄)haloalkyl, (C₁-C₄)alkoxy and (C₁-C₄)haloalkoxy.

In one embodiment R^{4A} is hydrogen, —CF₃ or phenyl.

In one embodiment R^{4A} is hydrogen.

One embodiment provides a bacterial efflux pump inhibitor which is a compound of formula IIb:

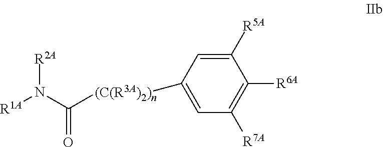

or a pharmaceutically acceptable salt thereof.

In one embodiment R^{7A} is hydrogen.

One embodiment provides a bacterial efflux pump inhibitor which is a compound of formula IIc:

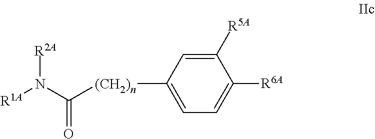

or a pharmaceutically acceptable salt thereof.

In one embodiment R^{5A} is halo, (C₁-C₆)alkyl, (C₁-C₆)haloalkyl, (C₁-C₆)alkoxy, (C₁-C₄)haloalkoxy, aryl or heteroaryl wherein the aryl or heteroaryl is optionally substituted with one or more (e.g., 1, 2, 3, 4 or 5) groups independently selected from halo, (C₁-C₄)alkyl, (C₁-C₄)haloalkyl, (C₁-C₄)alkoxy and (C₁-C₄)haloalkoxy;

In one embodiment R^{5A} is (C₁-C₆)haloalkyl.

In one embodiment R^{5A} —CF₃.

In one embodiment R^{6A} is halo, (C₁-C₆)alkyl, (C₁-C₆)haloalkyl, (C₁-C₆)alkoxy, (C₁-C₄)haloalkoxy, aryl or heteroaryl wherein the aryl or heteroaryl is optionally substituted with one or more (e.g., 1, 2, 3, 4 or 5) groups independently selected from halo, (C₁-C₄)alkyl, (C₁-C₄)haloalkyl, (C₁-C₄)alkoxy and (C₁-C₄)haloalkoxy;

In one embodiment R^{6A} is (C₁-C₆)haloalkyl.

In one embodiment R^{6A} —CF₃.

In one embodiment R^{6A} is hydrogen.

One embodiment provides a bacterial efflux pump inhibitor which is a compound formula IId:

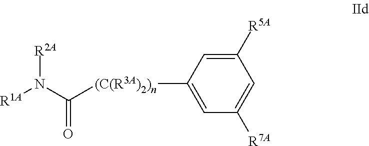

or a pharmaceutically acceptable salt thereof.

One embodiment provides a bacterial efflux pump inhibitor which is a compound of formula IIe:

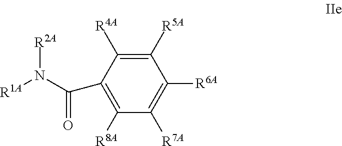

or a pharmaceutically acceptable salt thereof.

One embodiment provides a bacterial efflux pump inhibitor which is a compound of formula IIf:

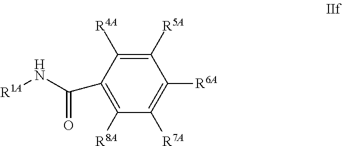

or a pharmaceutically acceptable salt thereof.

One embodiment provides a bacterial efflux pump inhibitor which is a compound of formula IIg:

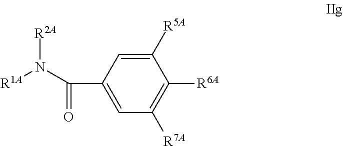

or a pharmaceutically acceptable salt thereof.

In one embodiment R^{5A} is halo, (C₁-C₆)alkyl, (C₁-C₆)haloalkyl, (C₁-C₆)alkoxy, (C₁-C₄)haloalkoxy, aryl or heteroaryl wherein the aryl or heteroaryl is optionally substituted with one or more (e.g., 1, 2, 3, 4 or 5) groups independently selected from halo, (C₁-C₄)alkyl, (C₁-C₄)haloalkyl, (C₁-C₄)alkoxy and (C₁-C₄)haloalkoxy.

In one embodiment R^{5A} is (C₁-C₆)alkyl, (C₁-C₆)haloalkyl, or aryl wherein the aryl is optionally substituted with one or more (e.g., 1, 2, 3, 4 or 5) groups independently selected from halo, (C₁-C₄)alkyl, (C₁-C₄)haloalkyl, (C₁-C₄)alkoxy and (C₁-C₄)haloalkoxy.

In one embodiment R^{5A} is (C₁-C₆)alkyl, (C₁-C₆)haloalkyl, or phenyl wherein phenyl is optionally substituted with one or more (e.g., 1, 2, 3, 4 or 5) groups independently selected from halo, (C₁-C₄)alkyl, (C₁-C₄)haloalkyl, (C₁-C₄)alkoxy and (C₁-C₄)haloalkoxy.

In one embodiment R^{5A} is tert-butyl, —CF₃, phenyl or 4-fluorophenyl.

In one embodiment R^{7A} is halo, (C₁-C₆)alkyl, (C₁-C₆)haloalkyl, (C₁-C₆)alkoxy, (C₁-C₄)haloalkoxy, aryl or heteroaryl wherein the aryl or heteroaryl is optionally substituted with one or more (e.g., 1, 2, 3, 4 or 5) groups independently selected from halo, (C₁-C₄)alkyl, (C₁-C₄)haloalkyl, (C₁-C₄)alkoxy and (C₁-C₄)haloalkoxy.

In one embodiment R^{7A} is (C₁-C₆)alkyl, (C₁-C₆)haloalkyl or aryl wherein the aryl is optionally substituted with one or more (e.g., 1, 2, 3, 4 or 5) groups independently selected from halo, (C₁-C₄)alkyl, (C₁-C₄)haloalkyl, (C₁-C₄)alkoxy and (C₁-C₄)haloalkoxy.

In one embodiment R^{7A} is (C₁-C₆)alkyl, (C₁-C₆)haloalkyl, or phenyl wherein phenyl is optionally substituted with one or more (e.g., 1, 2, 3, 4 or 5) groups independently selected from halo, (C₁-C₄)alkyl, (C₁-C₄)haloalkyl, (C₁-C₄)alkoxy and (C₁-C₄)haloalkoxy.

In one embodiment R^{7A} is tert-butyl, —CF₃, phenyl or 4-fluorophenyl.

In one embodiment the moiety:

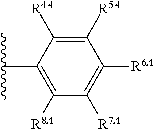

of the compound of formula II is:

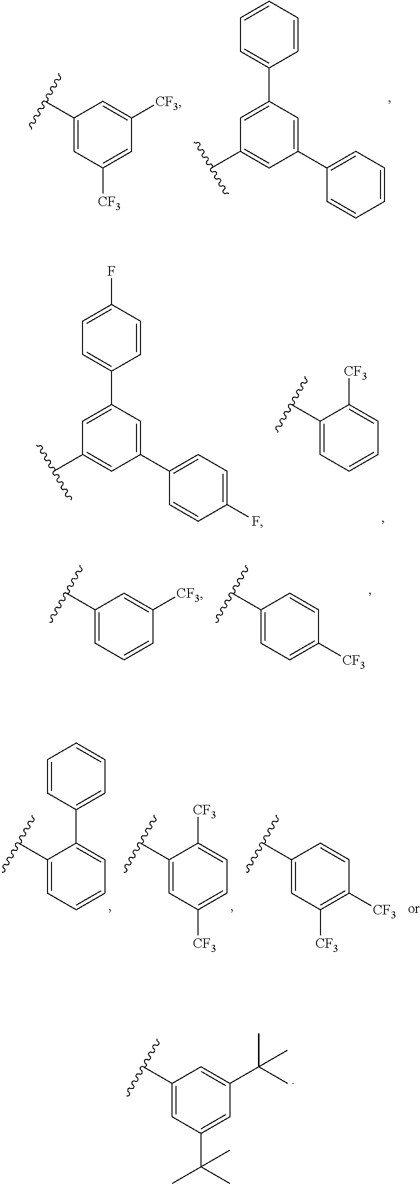

In one embodiment R^{1A} is (C₃-C₈)alkyl substituted with two or more groups independently selected from —NR^{b1}R^{c1}.

In one embodiment R^{1A} is (C₃-C₈)alkyl substituted with two groups independently selected from —NR^{b1}R^{c1}.

In one embodiment R^{1A} is (C₄-C₅)alkyl substituted with two groups independently selected from —NR^{b1}R^{c1}.

In one embodiment R^{b1} and R^{c1} are each hydrogen.

In one embodiment R^{1A} is:

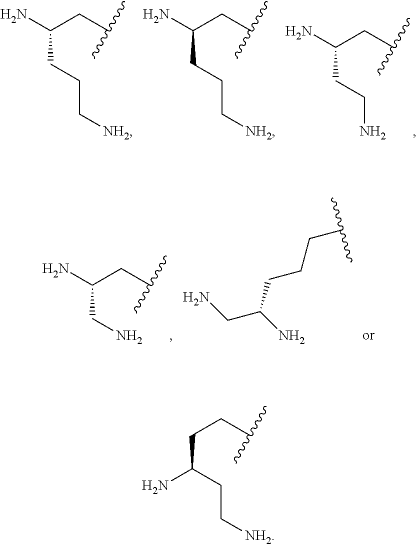 [SMILES: *C[C@@H](N)CCCN ; *C[C@H](N)CCCN ; *C[C@@H](N)CCN ; *C[C@@H](N)CN ; *CCC[C@H](N)CN ; *CC[C@H](N)CCN]

In one embodiment R^{1A} is:

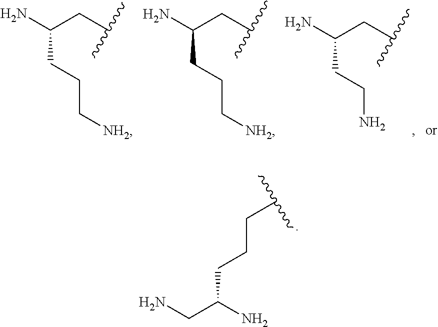 [SMILES: *C[C@@H](N)CCCN ; *C[C@H](N)CCCN ; *C[C@@H](N)CCN ; *CCC[C@H](N)CN]

In one embodiment n is 0.

In one embodiment, the bacterial efflux pump inhibitor is a compound of formula III:

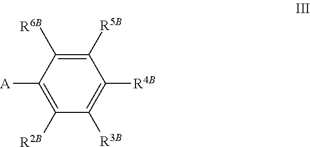

or a pharmaceutically acceptable salt thereof, wherein:

A is —C(═O)N(R^{a1})—R^{1B}, —(C₁-C₃)alkyl-C(═O)N(R^{a1})R^{1B}, —(C₁-C₃)alkyl-O—R^{1B}, —O—R^{1B}, —(C₁-C₃)alkyl-N(R^{a1})—R^{1B}, or —N(R^{a1})—R^{1B};

each R^{1B} is independently a (C₃-C₇)carbocyclyl, (C₃-C₇)carbocyclyl-(C₁-C₄)alkyl-, 4-7 membered monocyclic heterocyclyl, or 4-7 membered monocyclic heterocyclyl-(C₁-C₄)alkyl-, wherein each (C₃-C₇)carbocyclyl or (C₃-C₇)carbocyclyl-(C₁-C₄)alkyl- is independently substituted with one or more groups selected from the group consisting of Z and —(C₁-C₆)alkyl substituted with one or more Z, and wherein each 4-7 membered monocyclic heterocyclyl or 4-7 membered monocyclic heterocyclyl-(C₁-C₄)alkyl- is independently optionally substituted with one or more groups selected from the group consisting of Z and —(C₁-C₆)alkyl substituted with one or more Z, wherein each Z is independently selected from the group consisting of NR^{b2}R^{c2}, —NHNH₂, —C(═NR^{a2})(NR^{b2}R^{c2}), —NR^{a2}C(═NR^{a2})(R^{d2}), and —NR^{a2}C(═NR^{a2})(NR^{b2}R^{c2}) and wherein each (C₃-C₇)carbocyclyl, (C₃-C₇)carbocyclyl-(C₁-C₄)alkyl-, 4-7 membered monocyclic heterocyclyl, or 4-7 membered monocyclic heterocyclyl-(C₁-C₄)alkyl-, is independently optionally substituted independently with one or more (C₁-C₄)alkyl;

R^{2B} is hydrogen, halo, (C₁-C₄)alkyl, (C₁-C₄)haloalkyl, (C₁-C₄)alkoxy, (C₁-C₄)haloalkoxy, aryl or heteroaryl wherein the aryl or heteroaryl is optionally substituted with one or more groups independently selected from halo, —NO₂, —CN, (C₁-C₄)alkyl, (C₁-C₄)haloalkyl, (C₁-C₄)alkoxy and (C₁-C₄)haloalkoxy;

R^{3B} is hydrogen, halo, (C₁-C₆)alkyl, (C₁-C₄)haloalkyl, (C₁-C₆)alkoxy, (C₁-C₄)haloalkoxy, aryl or heteroaryl wherein the aryl or heteroaryl is optionally substituted with one or more groups independently selected from halo, —NO₂, —CN, (C₁-C₆)alkyl, (C₁-C₄)haloalkyl, (C₁-C₆)alkoxy and (C₁-C₄)haloalkoxy;

R^{4B} is hydrogen, halo, (C₁-C₆)alkyl, (C₁-C₄)haloalkyl, (C₁-C₆)alkoxy, (C₁-C₄)haloalkoxy, aryl or heteroaryl wherein the aryl or heteroaryl is optionally substituted with one or more groups independently selected from halo, —NO₂, —CN, (C₁-C₆)alkyl, (C₁-C₄)haloalkyl, (C₁-C₆)alkoxy and (C₁-C₄)haloalkoxy;

R^{5B} is hydrogen, halo, (C₁-C₆)alkyl, (C₁-C₄)haloalkyl, (C₁-C₆)alkoxy, (C₁-C₄)haloalkoxy, aryl or heteroaryl wherein the aryl or heteroaryl is optionally substituted with one or more groups independently selected from halo, —NO₂, —CN, (C₁-C₆)alkyl, (C₁-C₄)haloalkyl, (C₁-C₆)alkoxy and (C₁-C₄)haloalkoxy;

R^{6B} is hydrogen, halo, (C₁-C₄)alkyl, (C₁-C₄)haloalkyl, (C₁-C₄)alkoxy, (C₁-C₄)haloalkoxy, aryl or heteroaryl wherein the aryl or heteroaryl is optionally substituted with one or more groups independently selected from halo, —NO₂, —CN, (C₁-C₄)alkyl, (C₁-C₄)haloalkyl, (C₁-C₄)alkoxy and C₁-C₄)haloalkoxy;

each R^{a1} is independently hydrogen, (C₁-C₆)alkyl or (C₃-C₇)carbocyclyl;

each R^{a2} is independently hydrogen, (C₁-C₆)alkyl or (C₃-C₇)carbocyclyl;

each R^{b2} and R^{c2} is independently hydrogen, (C₁-C₆)alkyl or (C₃-C₇)carbocyclyl; and

R^{d2} is (C₁-C₆)alkyl or (C₃-C₇)carbocyclyl.

It is to understood that the embodiments provided below are for compounds of formula III and all sub-formulas thereof. It is to be understood the two or more embodiments may be combined.

In one embodiment A is —C(═O)N(R^{a1})—R^{1B}, —(C₁-C₃)alkyl-C(═O)N(R^{a1})R^{1B}, —(C₁-C₃)alkyl-O—R^{1B}, or —O—R^{1B}.

In one embodiment A is —C(═O)N(R^{a1})—R^{1B}.

In one embodiment A is —(C₁-C₃)alkyl-C(═O)N(R^{a1})R^{1B}.

In one embodiment R^{a1} is hydrogen.

In one embodiment A is —O—R^{1B}.

In one embodiment R^{2B} is hydrogen, halo, (C₁-C₄)alkyl, (C₁-C₄)haloalkyl, (C₁-C₄)alkoxy, or (C₁-C₄)haloalkoxy.

In one embodiment R^{2B} is hydrogen.

In one embodiment R^{3B} is aryl or heteroaryl wherein the aryl or heteroaryl is optionally substituted with one or more groups independently selected from halo, —NO₂, —CN, (C₁-C₆)alkyl, (C₁-C₄)haloalkyl, (C₁-C₆)alkoxy and (C₁-C₄)haloalkoxy.

In one embodiment R^{3B} is phenyl wherein the phenyl is optionally substituted with one or more groups independently selected from halo, —NO₂, —CN, (C₁-C₆)alkyl, (C₁-C₄)haloalkyl, (C₁-C₆)alkoxy and (C₁-C₄)haloalkoxy.

In one embodiment R^{3B} is 4-fluorophenyl.

In one embodiment R^{4B} is hydrogen, halo, (C₁-C₆)alkyl, (C₁-C₄)haloalkyl, (C₁-C₆)alkoxy, (C₁-C₄)haloalkoxy.

In one embodiment R^{4B} is hydrogen.

In one embodiment R^{5B} is aryl or heteroaryl wherein the aryl or heteroaryl is optionally substituted with one or more groups independently selected from halo, —NO₂, —CN, (C₁-C₆)alkyl, (C₁-C₄)haloalkyl, (C₁-C₆)alkoxy and (C₁-C₄)haloalkoxy.

In one embodiment R^{5B} is phenyl wherein the phenyl is optionally substituted with one or more groups independently selected from halo, —NO₂, —CN, (C₁-C₆)alkyl, (C₁-C₄)haloalkyl, (C₁-C₆)alkoxy and (C₁-C₄)haloalkoxy.

In one embodiment R^{5B} is 4-fluorophenyl.

In one embodiment R^{6B} is hydrogen, halo, (C₁-C₄)alkyl, (C₁-C₄)haloalkyl, (C₁-C₄)alkoxy, or (C₁-C₄)haloalkoxy.

In one embodiment R^{6B} is hydrogen.

In one embodiment the moiety:

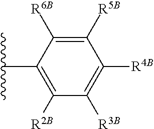

of the compound of formula III is:

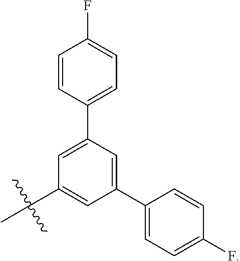 [SMILES: *c1cc(-c2ccc(F)cc2)cc(-c2ccc(F)cc2)c1]

In one embodiment R^{1B} is a 4-7 membered monocyclic heterocyclyl or 4-7 membered monocyclic heterocyclyl-(C₁-C₄)alkyl-, wherein the 4-7 membered monocyclic heterocyclyl or 4-7 membered monocyclic heterocyclyl-(C₁-C₄)alkyl- is optionally substituted independently with one or more groups selected from the group consisting of Z and —(C₁-C₆)alkyl substituted with one or more Z, wherein each Z is NR^{b2}R^{c2}nd wherein the 4-7 membered monocyclic heterocyclyl or 4-7 membered monocyclic heterocyclyl-(C₁-C₄)alkyl- is optionally substituted with one or more (C₁-C₄)alkyl.

In one embodiment R^{1B} is a 4-7 membered monocyclic N-heterocyclyl or 4-7 membered monocyclic N-heterocyclyl-(C₁-C₄)alkyl-, wherein the 4-7 membered monocyclic N-heterocyclyl or 4-7 membered monocyclic N-heterocyclyl-(C₁-C₄)alkyl- is optionally substituted independently with one or more groups selected from the group consisting of Z and —(C₁-C₆)alkyl substituted with one or more Z, wherein each Z is NR^{b2}R^{c2}nd wherein the 4-7 membered monocyclic N-heterocyclyl or 4-7 membered monocyclic N-heterocyclyl-(C₁-C₄)alkyl- is optionally substituted with one or more (C₁-C₄)alkyl.

In one embodiment R^{1B} is a pyrrolidinyl or pyrrolidinyl-(C₁-C₄)alkyl-, wherein the pyrrolidinyl or pyrrolidinyl-(C₁-C₄)alkyl- is optionally substituted independently with one or more groups selected from the group consisting of Z and —(C₁-C₆)alkyl substituted with one or more Z, wherein each Z is NR^{b2}R^{c2}nd wherein the pyrrolidinyl or pyrrolidinyl-(C₁-C₄)alkyl- is optionally substituted with one or more (C₁-C₄)alkyl.

In one embodiment R^{1B} is a pyrrolidinyl or pyrrolidinyl-(CH₂)—, wherein the pyrrolidinyl-(CH₂)— is optionally substituted independently with one or more groups selected from the group consisting of Z and —(C₁-C₆)alkyl substituted with one or more Z, wherein each Z is NR^{b2}R^{c2} and wherein the pyrrolidinyl or pyrrolidinyl-(C₁-C₄)alkyl- is optionally substituted with one or more (C₁-C₄)alkyl.

In one embodiment R^{1B} is a pyrrolidinyl or pyrrolidinyl-(CH₂)—, wherein the pyrrolidinyl-(CH₂)— is optionally substituted independently with one or more groups selected from the group consisting of Z and —(C₁-C₄)alkyl substituted with one or more Z, wherein each Z is NR^{b2}R^{c2}.

In one embodiment each R^{b2} and R^{c2} is hydrogen

In one embodiment R^{1B} is:

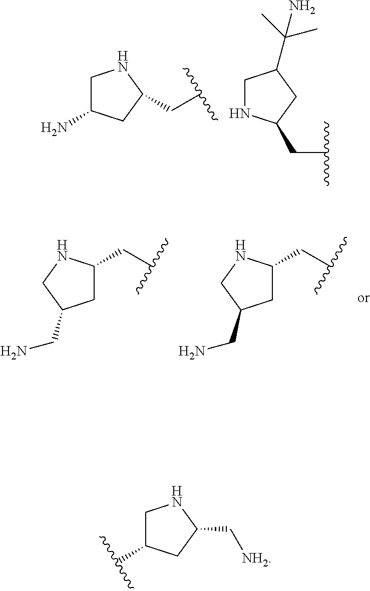

In one embodiment A is:

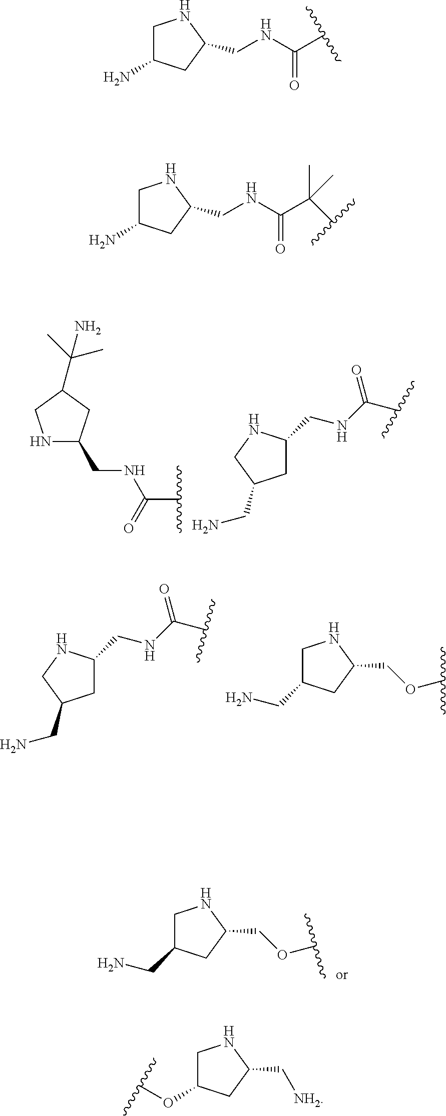

In one embodiment, the bacterial efflux pump inhibitor is a compound of formula IV:

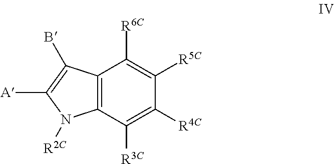

or a pharmaceutically acceptable salt thereof, wherein:

one of A′ or B′ is —C(═O)N(R^{a1})—R^{1C}, —(C₁-C₃)alkyl-C(═O)N(R^{a1})R^{1C}, —(C₁-C₃)alkyl-O—R^{1C}, —O—R^{1C}, —(C₁-C₃)alkyl-N(R^{a1})—R^{1C}, —N(R^{a1})—R^{1C}, or R^{1C} and the other of A′ or B′ is H, halogen, or (C₁-C₄)alkyl;

each R^{1C} is independently:

(a) (C₁-C₁₄)alkyl substituted with one or more groups selected from the group consisting of —NR^{b2}R^{c2}, —NHNH₂, —C(═NR^{a2})(NR^{b2}R^{c2}), —NR^{a2}C(═NR^{a2})(R^{d2}), and —NR^{a2}C(═NR^{a2})(NR^{b2}R^{c2}); and wherein (C₁-C₁₄)alkyl is optionally substituted independently with one or more halo, (C₁-C₄)alkyl or (C₃-C₇)carbocyclyl; or

(b) (C₃-C₇)carbocyclyl, (C₃-C₇)carbocyclyl-(C₁-C₄)alkyl-, 4-7 membered monocyclic heterocyclyl, 4-7 membered monocyclic heterocyclyl-(C₁-C₄)alkyl-, (C₃-C₇)carbocyclyl-NR^{e}—(C₁-C₄)alkyl- or 4-7 membered monocyclic heterocyclyl-NR^{e}—(C₁-C₄)alkyl- wherein each (C₃-C₇)carbocyclyl, (C₃-C₇)carbocyclyl-(C₁-C₄)alkyl- or —(C₃-C₇)carbocyclyl-NR^{e}—(C₁-C₄)alkyl- is independently substituted with one or more Z¹ or Z², and wherein each 4-7 membered monocyclic heterocyclyl, 4-7 membered monocyclic heterocyclyl-(C₁-C₄)alkyl- or 4-7 membered monocyclic heterocyclyl-NR^{e}—(C₁-C₄)alkyl- is independently optionally substituted with one or more Z¹ or Z², and wherein any (C₃-C₇)carbocyclyl, (C₃-C₇)carbocyclyl-(C₁-C₄)alkyl-, 4-7 membered monocyclic heterocyclyl, 4-7 membered monocyclic heterocyclyl-(C₁-C₄)alkyl-, (C₃-C₇)carbocyclyl NR^{e}—(C₁-C₄)alkyl- or 4-7 membered monocyclic heterocyclyl-NR^{e}—(C₁-C₄)alkyl- of R¹ is independently optionally substituted with one or more halo, (C₁-C₄)alkyl, (C₃-C₇)carbocyclyl, —C(═O)NH₂, —C(═O)NH(C₁-C₄)alkyl, —C(═O)N((C₁-C₄)alkyl)₂, —NHC(═O)(C₁-C₄)alkyl-NH₂, or 3-7 membered monocyclic heterocyclyl wherein (C₁-C₄)alkyl, (C₃-C₇)carbocyclyl or 3-7 membered monocyclic heterocyclyl is optionally substituted with one or more halogen, (C₁-C₄)alkyl, —NH₂, —NH(C₁-C₄)alkyl or —N((C₁-C₄)alkyl)₂;

R^{2C} is hydrogen, (C₁-C₄)alkyl or phenyl(C₁-C₃)alkyl-, wherein the phenyl is optionally substituted with one or more (C₁-C₄)alkyl, —O(C₁-C₄)alkyl, halogen, or —NO₂;

R^{3C} is hydrogen, halo, (C₁-C₄)alkyl, (C₁-C₄)haloalkyl, (C₁-C₄)alkoxy, (C₁-C₄)haloalkoxy, aryl, or heteroaryl wherein the aryl or heteroaryl is optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy;

R^{4C} is hydrogen, halo, (C₁-C₄)alkyl, (C₁-C₄)haloalkyl, (C₁-C₄)alkoxy, (C₁-C₄)haloalkoxy, aryl, heteroaryl, aryl(C₁-C₄)alkyl-, heteroaryl(C₁-C₄)alkyl-, (C₃-C₇)carbocyclyl(C₁-C₄)alkyl-, (C₃-C₇)carbocyclyl(C₂-C₄)alkynyl-, phenoxy or heteroaryloxy, wherein the aryl, heteroaryl, aryl(C₁-C₄)alkyl-, heteroaryl(C₁-C₄)alkyl-, (C₃-C₇)carbocyclyl(C₁-C₄)alkyl-, (C₃-C₇)carbocyclyl(C₂-C₄)alkynyl-, phenoxy or heteroaryloxy, is optionally substituted with one or more groups independently selected from the group consisting of halo, —OH, —NO₂, —CN, (C₁-C₄)alkyl, (C₁-C₄)haloalkyl, (C₁-C₄)alkoxy, (C₁-C₄)haloalkoxy, methylenedioxy (—OCH₂O—), and (C₃-C₇)carbocyclyl; R^{5C} is hydrogen, halo, (C₁-C₄)alkyl, (C₁-C₄)haloalkyl, (C₁-C₄)alkoxy, (C₁-C₄)haloalkoxy, aryl, heteroaryl aryl(C₁-C₄)alkyl-, heteroaryl(C₁-C₄)alkyl-, (C₃-C₇)carbocyclyl(C₁-C₄)alkyl-, (C₃-C₇)carbocyclyl(C₂-C₄)alkynyl-, phenoxy or heteroaryloxy, wherein the aryl, heteroaryl, aryl(C₁-C₄)alkyl-, heteroaryl(C₁-C₄)alkyl-, (C₃-C₇)carbocyclyl(C₁-C₄)alkyl-, (C₃-C₇)carbocyclyl(C₂-C₄)alkynyl-, phenoxy or heteroaryloxy, is optionally substituted with one or more groups independently selected from the group consisting of halo, —OH, —NO₂, —CN, (C₁-C₄)alkyl, (C₁-C₄)haloalkyl, (C₁-C₄)alkoxy, (C₁-C₄)haloalkoxy, methylenedioxy (—OCH₂O—), and (C₃-C₇)carbocyclyl;

R^{6C} is hydrogen, halo, (C₁-C₄)alkyl, (C₁-C₄)haloalkyl, (C₁-C₄)alkoxy, (C₁-C₄)haloalkoxy, aryl, or heteroaryl wherein the aryl or heteroaryl is optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy;

each Z¹ is independently selected from the group consisting of —NR^{b3}R^{c3}, —NHNH₂, —C(═NR^{a3})(NR^{b3}R^{c3}), —NR^{a3}C(═NR^{a3})(R^{d3}), and —NR^{a3}C(═NR^{a3})(NR^{b3}R^{c3});

each Z² is independently —(C₁-C₆)alkyl substituted with one or more Z¹ and optionally substituted with one or more Z³;

each Z³ is independently halo or (C₃-C₇)carbocyclyl;

each R^{a1} is independently hydrogen, (C₁-C₄)alkyl, (C₃-C₇)carbocyclyl or 3-7 membered monocyclic heterocycly optionally substituted with one or more halogen or (C₁-C₄)alkyl;

each R^{a2} is independently hydrogen, (C₁-C₄)alkyl or (C₃-C₇)carbocyclyl;

each R^{b2} and R^{c2} is independently hydrogen, (C₁-C₄)alkyl or (C₃-C₇)carbocyclyl;

R^{d2} is (C₁-C₄)alkyl or (C₃-C₇)carbocyclyl;

each R^{a3} is independently hydrogen (C₁-C₄)alkyl or (C₃-C₇)carbocyclyl;

each R^{b3} and R³ is independently hydrogen (C₁-C₄)alkyl or (C₃-C₇)carbocyclyl;

R^{d3} is (C₁-C₄)alkyl or (C₃-C₇)carbocyclyl; and

each R^{e} is independently hydrogen, (C₁-C₄)alkyl or (C₃-C₇)carbocyclyl.

In one embodiment, the bacterial efflux pump inhibitor is a compound of formula IV:

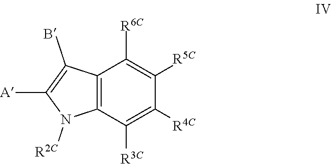

or a pharmaceutically acceptable salt thereof, wherein:

one of A′ or B′ is —C(═O)N(R^{a1})—R^{1c}, —(C₁-C₃)alkyl-C(═O)N(R^{a1})R^{1C}, —(C₁-C₃)alkyl-O—R^{1C}, —O—R^{1C}, —(C₁-C₃)alkyl-N(R^{a1})—R^{C}, —N(R^{a1})—R^{1C}, or R^{1C} and the other of A′ or B′ is H, halogen, or (C₁-C₄)alkyl;

each R^{1C} is independently:

(a) (C₁-C₁₄)alkyl substituted with one or more groups selected from the group consisting of —NR^{b2}R^{c2}, —NHNH₂, —C(═NR^{a2})(NR^{b2}R^{c2}), —NR^{a2}C(═NR^{a2})(R^{d2}), and —NR^{a2}C(═NR^{a2})(NR^{b2}R^{c2}); and wherein (C₁-C₁₄)alkyl is optionally substituted independently with one or more halo, (C₁-C₄)alkyl or (C₃-C₇)carbocyclyl; or

(b) (C₃-C₇)carbocyclyl, (C₃-C₇)carbocyclyl-(C₁-C₄)alkyl-, 4-7 membered monocyclic heterocyclyl, or 4-7 membered monocyclic heterocyclyl-(C₁-C₄)alkyl-, wherein each (C₃-C₇)carbocyclyl or (C₃-C₇)carbocyclyl-(C₁-C₄)alkyl- is independently substituted with one or more Z¹ or Z², and wherein each 4-7 membered monocyclic heterocyclyl or 4-7 membered monocyclic heterocyclyl-(C₁-C₄)alkyl- is independently optionally substituted with one or more Z¹ or Z², and wherein any (C₃-C₇)carbocyclyl, (C₃-C₇)carbocyclyl-(C₁-C₄)alkyl-, 4-7 membered monocyclic heterocyclyl, or 4-7 membered monocyclic heterocyclyl-(C₁-C₄)alkyl- of R¹ is independently optionally substituted independently with one or more halo, (C₁-C₄)alkyl or (C₃-C₇)carbocyclyl;

R^{2C} is hydrogen, (C₁-C₄)alkyl or phenyl(C₁-C₃)alkyl-, wherein the phenyl is optionally substituted with one or more (C₁-C₄)alkyl, —O(C₁-C₄)alkyl, halogen, or —NO₂;

R^{3C} is hydrogen, halo, (C₁-C₄)alkyl, (C₁-C₄)haloalkyl, (C₁-C₄)alkoxy, (C₁-C₄)haloalkoxy, aryl, or heteroaryl wherein the aryl or heteroaryl is optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy;

R^{4C} is hydrogen, halo, (C₁-C₄)alkyl, (C₁-C₄)haloalkyl, (C₁-C₄)alkoxy, (C₁-C₄)haloalkoxy, aryl, or heteroaryl wherein the aryl or heteroaryl is optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy;

R^{5C} is hydrogen, halo, (C₁-C₄)alkyl, (C₁-C₄)haloalkyl, (C₁-C₄)alkoxy, (C₁-C₄)haloalkoxy, aryl, or heteroaryl wherein the aryl or heteroaryl is optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy;

R^{6C} is hydrogen, halo, (C₁-C₄)alkyl, (C₁-C₄)haloalkyl, (C₁-C₄)alkoxy, (C₁-C₄)haloalkoxy, aryl, or heteroaryl wherein the aryl or heteroaryl is optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy;

each Z¹ is independently selected from the group consisting of —NR^{b3}R^{c3}, —NHNH₂, —C(═NR^{a3})(NR^{b3}R^{c3}), —NR^{a3}C(═NR^{a3})(R^{d3}), and —NR^{a3}C(═NR^{a3})(NR^{b3}R^{c3});

each Z² is independently —(C₁-C₆)alkyl substituted with one or more Z¹ and optionally substituted with one or more Z³;

each Z³ is independently halo or (C₃-C₇)carbocyclyl;

each R^{a1} is independently hydrogen, (C₁-C₄)alkyl or (C₃-C₇)carbocyclyl;

each R^{a2} is independently hydrogen, (C₁-C₄)alkyl or (C₃-C₇)carbocyclyl;

each R^{b2} and R^{c2} is independently hydrogen, (C₁-C₄)alkyl or (C₃-C₇)carbocyclyl;

R^{d2} is (C₁-C₄)alkyl or (C₃-C₇)carbocyclyl;

each R^{a3} is independently hydrogen (C₁-C₄)alkyl or (C₃-C₇)carbocyclyl;

each R^{b3} and R³ is independently hydrogen (C₁-C₄)alkyl or (C₃-C₇)carbocyclyl; and

R^{d3} is (C₁-C₄)alkyl or (C₃-C₇)carbocyclyl.

It is to understood that the embodiments provided below are for compounds of formula IV and all sub-formulas thereof (e.g., formulas IVa, IVb). It is to be understood the two or more embodiments may be combined.

In one embodiment one of A′ or B′ is —C(═O)N(R^{a1})—R^{1C} or —(C₁-C₃)alkyl-C(═O)N(R^{a1})R^{1C}, and the other of A′ or B′ is H, halogen, or (C₁-C₆)alkyl.

In one embodiment one of A′ or B′ is —C(═O)N(R^{a1})—R^{1C}, and the other of A′ or B′ is H, halogen, or (C₁-C₆)alkyl.

In one embodiment A′ is —C(═O)N(R^{a1})—R^{1C}, and B′ is H.

In one embodiment B′ is —C(═O)N(R^{a1})—R^{1C}, and A′ is H.

In one embodiment one of A′ or B′ is —C(═O)N(R^{a1})—R^{1C} or —(C₁-C₃)alkyl-C(═O)N(R^{a1})R^{1C}, and the other of A′ or B′ is H.

In one embodiment one of A′ or B′ is —C(═O)N(R^{a1})—R^{1C}, and the other of A′ or B′ is H.

In one embodiment R^{2C} is hydrogen, (C₁-C₄)alkyl or benzyl, wherein benzyl is optionally substituted with one or more (C₁-C₄)alkyl, —O(C₁-C₄)alkyl, halogen or —NO₂.

In one embodiment R^{a1} is hydrogen.

In one embodiment R^{2C} is hydrogen or (C₁-C₄)alkyl.

In one embodiment R^{2C} is hydrogen.

In one embodiment R^{2C} is hydrogen, methyl, or 4-fluorobenzyl.

In one embodiment a compound of formula IV is a compound formula IVa or IVb:

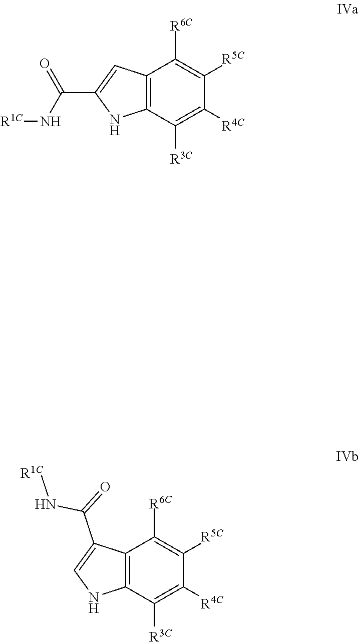

or a pharmaceutically acceptable salt thereof.

In one embodiment R^{3C} is hydrogen or aryl wherein the aryl is optionally substituted with one or more groups independently selected from the group consisting of halo, —OH, —NO₂, —CN, (C₁-C₄)alkyl, (C₁-C₄)haloalkyl, (C₁-C₄)alkoxy and (C₁-C₄)haloalkoxy.

In one embodiment R^{3C} is hydrogen or phenyl wherein the phenyl is optionally substituted with one or more groups independently selected from the group consisting of halo, —OH, —NO₂, —CN, (C₁-C₄)alkyl, (C₁-C₄)haloalkyl, (C₁-C₄)alkoxy and (C₁-C₄)haloalkoxy.

In one embodiment R^{3C} is hydrogen or 4-fluorophenyl.

In one embodiment R^{4C} is hydrogen, aryl, or heteroaryl wherein the aryl or heteroaryl is optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy.

In one embodiment R^{4C} is hydrogen, phenyl, or pyridinyl wherein the phenyl or pyridinyl is optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy.

In one embodiment R^{4C} is hydrogen, 4-nitrophenyl, 4-fluorophenyl, 3,4-difluorophenyl, 4-t-butylphenyl, 4-methoxyphenyl, pyridin-4-yl, 4-hydroxyphenyl, 4-chlorophenyl, or 4-cyanophenyl.

In one embodiment R^{5C} is hydrogen or aryl wherein the aryl is optionally substituted with one or more groups independently selected from the group consisting of halo, —OH, —NO₂, —CN, (C₁-C₄)alkyl, (C₁-C₄)haloalkyl, (C₁-C₄)alkoxy and (C₁-C₄)haloalkoxy.

In one embodiment R^{5C} is hydrogen or phenyl wherein the phenyl is optionally substituted with one or more groups independently selected from the group consisting of halo, —OH, —NO₂, —CN, (C₁-C₄)alkyl, (C₁-C₄)haloalkyl, (C₁-C₄)alkoxy and (C₁-C₄)haloalkoxy.

In one embodiment R^{5C} is hydrogen or 4-fluorophenyl.

In one embodiment R^{6C} is hydrogen or aryl wherein the aryl is optionally substituted with one or more groups independently selected from the group consisting of halo, —OH, —NO₂, —CN, (C₁-C₄)alkyl, (C₁-C₄)haloalkyl, (C₁-C₄)alkoxy and (C₁-C₄)haloalkoxy.

In one embodiment R^{6C} is hydrogen or phenyl wherein the phenyl is optionally substituted with one or more groups independently selected from the group consisting of halo, —OH, —NO₂, —CN, (C₁-C₄)alkyl, (C₁-C₄)haloalkyl, (C₁-C₄)alkoxy and (C₁-C₄)haloalkoxy.

In one embodiment R^{6C} is hydrogen or 4-fluorophenyl.

In one embodiment R^{1C} is (C₁-C₁₄)alkyl substituted with one or more groups independently selected from —NR^{b2}R^{c2}.

In one embodiment R^{1C} is (C₂-C₁₀)alkyl substituted with one or more groups independently selected from —NR^{b2}R^{c2}.

In one embodiment R^{1C} is (C₁-C₁₄)alkyl substituted with one or more groups independently selected from —NR^{b2}R^{c2}.

In one embodiment R^{1C} is (C₂-C₈)alkyl substituted with two or more groups independently selected from —NR^{b2}R^{c2}.

In one embodiment R^{1C} is (C₄-C₅)alkyl substituted with two or more groups independently selected from —NR^{b2}R^{c2}.

In one embodiment R^{b2} and R^{c2} are each hydrogen.

In one embodiment R^{1C} is a 4-7 membered monocyclic heterocyclyl-(C₁-C₄)alkyl-, wherein the 4-7 membered monocyclic heterocyclyl-(C₁-C₄)alkyl- is substituted with one or more groups independently selected from the group consisting of Z and —(C₁-C₆)alkyl substituted with one or more Z, wherein each Z is independently selected from the group consisting of —NR^{b3}R^{c3}, —NHNH₂, —C(═NR^{a3})(NR^{b3}R^{c3}), —NR^{a3}C(═NR^{a3})(R^{d3}), and NR^{a3}C(═NR^{a3})(NR^{b3}R^{c3}) and wherein the 4-7 membered monocyclic heterocyclyl-(C₁-C₄)alkyl- is optionally substituted with one or more (C₁-C₆)alkyl.

In one embodiment R^{1C} is a 4-7 membered monocyclic heterocyclyl-(C₁-C₄)alkyl-, wherein the 4-7 membered monocyclic heterocyclyl-(C₁-C₄)alkyl- is substituted with one or more groups independently selected from the group consisting of Z and (C₁-C₆)alkyl substituted with one or more Z, wherein each Z is independently —NR^{b3}R^{c3} and wherein the 4-7 membered monocyclic heterocyclyl-(C₁-C₄)alkyl- is optionally substituted with one or more (C₁-C₆)alkyl.

In one embodiment R^{1C} is pyrrolidinyl-(C₁-C₄)alkyl-, wherein the pyrrolidinyl-(C₁-C₄)alkyl- is substituted with one or more groups independently selected from the group consisting of Z and —(C₁-C₆)alkyl substituted with one or more Z, wherein each Z is independently —NR^{b3}R^{c3} and wherein is pyrrolidinyl-(C₁-C₄)alkyl- is optionally substituted independently with one or more (C₁-C₆)alkyl.

In one embodiment R^{1C} is pyrrolidinyl-(CH₂)—, wherein the pyrrolidinyl-(CH₂)— is substituted with one or more groups independently selected from the group consisting of Z and —(C₁-C₆)alkyl substituted with one or more Z, wherein each Z is independently —NR^{b3}R^{c3} and wherein the pyrrolidinyl-(CH₂)— is optionally substituted independently with one or more (C₁-C₆)alkyl.

In one embodiment R^{1C} is pyrrolidinyl-(CH₂)—, wherein the pyrrolidinyl-(CH₂)— is substituted on the pyrrolidinyl with an —(C₁-C₆)alkyl substituted with one or more —NR^{b3}R^{c3}.

In one embodiment R^{b3} and R³ are each hydrogen.

In one embodiment R^{1C} is:

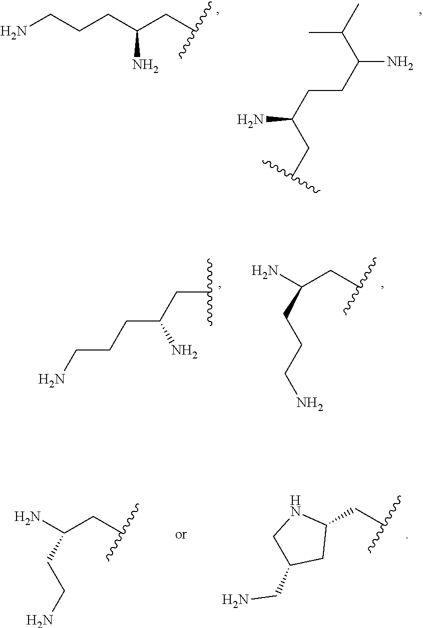 [SMILES: *C[C@@H](N)CCCN ; *C[C@@H](N)CCC(N)C(C)C ; *C[C@H](N)CCCN ; *C[C@H](N)CCCN ; *C[C@@H](N)CCN ; *C[C@@H]1C[C@H](CN)CN1]

In one embodiment one of A′ or B′ is:

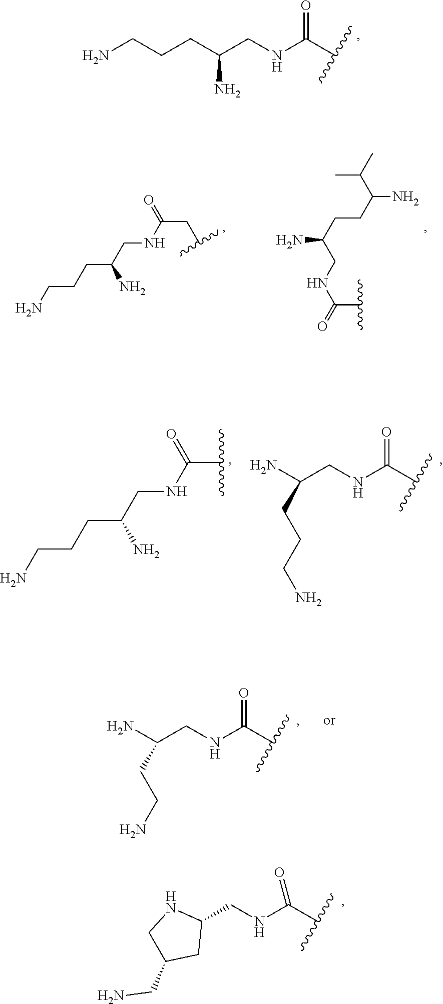

and the other of A′ or B′ is H.

In one embodiment, the bacterial efflux pump inhibitor is a compound of formula V:

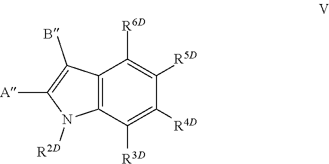

or a pharmaceutically acceptable salt thereof, wherein:

A″ is —C(═O)N(R^{a1})—R^{1D}, —(C₁-C₃)alkyl-C(═O)N(R^{a1})R^{1D}, —(C₁-C₃)alkyl-O—R^{1D}, —O—R^{1D}, —(C₁-C₃)alkyl-N(R^{a1})—R^{1D}, —N(R^{a1})—R^{1D}, or R_{1D};

B″ is (C₂-C₈)alkenyl, (C₂-C₈)alkynyl, (C₁-C₄)haloalkyl, (C₁-C₄)alkoxy, (C₁-C₄)haloalkoxy, (C₃-C₇)carbocyclyl, (C₃-C₇)carbocyclyl-(C₁-C₄)alkyl-, aryl, aryl-(C₁-C₄)alkyl-, heteroaryl, heteroaryl-(C₁-C₄)alkyl-, 3-7 membered-monocyclic-heterocycle, or 3-7 membered-monocyclic-heterocycle-(C₁-C₄)alkyl- wherein any (C₃-C₇)carbocyclyl, (C₃-C₇)carbocyclyl-(C₁-C₄)alkyl-, aryl, aryl-(C₁-C₄)alkyl-, heteroaryl, heteroaryl-(C₁-C₄)alkyl-, 3-7 membered-monocyclic-heterocycle, or 3-7 membered-monocyclic-heterocycle-(C₁-C₄)alkyl- of B″ is optionally substituted with one or more Z¹ groups;

each R^{1D} is independently:

(a) (C₁-C₁₄)alkyl substituted with one or more groups selected from the group consisting of —NR^{b2}R^{c2}, —NHNH₂, —C(═NR^{a2})(NR^{b2}R^{c2}), —NR^{a2}C(═NR^{a2})(R^{d2}), and —NR^{a2}C(═NR^{a2})(NR^{b2}R^{c2}) and wherein (C₁-C₁₄)alkyl is optionally substituted independently with one or more halo, (C₁-C₄)alkyl or (C₃-C₇)carbocyclyl; or

(b) (C₃-C₇)carbocyclyl, (C₃-C₇)carbocyclyl-(C₁-C₄)alkyl-, 4-7 membered monocyclic heterocyclyl, or 4-7 membered monocyclic heterocyclyl-(C₁-C₄)alkyl-, wherein each (C₃-C₇)carbocyclyl or (C₃-C₇)carbocyclyl-(C₁-C₄)alkyl- is independently substituted with one or more Z² or Z³, and wherein each 4-7 membered monocyclic heterocyclyl or 4-7 membered monocyclic heterocyclyl-(C₁-C₄)alkyl- is independently optionally substituted with one or more Z² or Z³, and wherein any (C₃-C₇)carbocyclyl, (C₃-C₇)carbocyclyl-(C₁-C₄)alkyl-, 4-7 membered monocyclic heterocyclyl, or 4-7 membered monocyclic heterocyclyl-(C₁-C₄)alkyl- of R¹ is optionally substituted independently with one or more halo, (C₁-C₄)alkyl or (C₃-C₇)carbocyclyl;

R^{2D} is hydrogen, (C₁-C₄)alkyl or phenyl(C₁-C₃)alkyl-, wherein the phenyl is optionally substituted with one or more (C₁-C₄)alkyl, —O(C₁-C₄)alkyl, halogen, or —NO₂;

R^{3D} is hydrogen, halo, (C₁-C₄)alkyl, (C₁-C₄)haloalkyl, (C₁-C₄)alkoxy, (C₁-C₄)haloalkoxy, aryl, or heteroaryl wherein the aryl or heteroaryl is optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy;

R^{4D} is hydrogen, halo, (C₁-C₄)alkyl, (C₁-C₄)haloalkyl, (C₁-C₄)alkoxy, (C₁-C₄)haloalkoxy, aryl, or heteroaryl wherein the aryl or heteroaryl is optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy;

R^{5D} is hydrogen, halo, (C₁-C₄)alkyl, (C₁-C₄)haloalkyl, (C₁-C₄)alkoxy, (C₁-C₄)haloalkoxy, aryl, or heteroaryl wherein the aryl or heteroaryl is optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy;

R^{6D} is hydrogen, halo, (C₁-C₄)alkyl, (C₁-C₄)haloalkyl, (C₁-C₄)alkoxy, (C₁-C₄)haloalkoxy, aryl, or heteroaryl wherein the aryl or heteroaryl is optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy;

each Z¹ is independently halo, —OH, —NO₂, —CN, (C₁-C₄)alkyl, (C₁-C₄)haloalkyl, (C₁-C₄)alkoxy, or (C₁-C₄)haloalkoxy;

each Z² is independently selected from the group consisting of —NR^{b3}R^{c3}, —NHNH₂, —C(═NR^{a3})(NR^{b3}R^{c3}), —NR^{a3}C(═NR^{a3})(R^{d3}), and —NR^{a3}C(═NR^{a3})(NR^{b3}R^{c3})

each Z³ is independently —(C₁-C₆)alkyl substituted with one or more Z² and optionally substituted with one or more Z⁴;

each Z⁴ is independently halo or (C₃-C₇)carbocyclyl;

each R^{a1} is independently hydrogen, (C₁-C₄)alkyl or (C₃-C₇)carbocyclyl;

each R^{a2} is independently hydrogen, (C₁-C₄)alkyl or (C₃-C₇)carbocyclyl;

each R^{b2} and R^{c2} is independently hydrogen, (C₁-C₄)alkyl or (C₃-C₇)carbocyclyl;

R^{d2} is (C₁-C₄)alkyl or (C₃-C₇)carbocyclyl;

each R^{a3} is independently hydrogen (C₁-C₄)alkyl or (C₃-C₇)carbocyclyl;

each R^{b3} and R³ is independently hydrogen (C₁-C₄)alkyl or (C₃-C₇)carbocyclyl; and

R^{d3} is (C₁-C₄)alkyl or (C₃-C₇)carbocyclyl.

It is understood that the embodiments provided below are for compounds of formula V and all sub-formulas thereof (e.g., formulas Va). It is to be understood the two or more embodiments may be combined.

In one embodiment A″ is —C(═O)N(R^{a1})—R^{1D}.

In one embodiment R^{a1} is hydrogen.

In one embodiment R^{2D} is hydrogen or (C₁-C₆)alkyl.

In one embodiment R^{2D} is hydrogen.

In one embodiment a compound of formula I is a compound of formula Va:

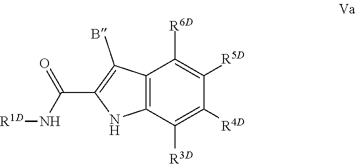

or a pharmaceutically acceptable salt thereof.

In one embodiment R^{3D} is hydrogen, halo, (C₁-C₄)alkyl, (C₁-C₄)haloalkyl, (C₁-C₄)alkoxy, or (C₁-C₄)haloalkoxy.

In one embodiment R^{3D} is hydrogen.

In one embodiment R^{4D} is hydrogen, aryl, or heteroaryl wherein the aryl or heteroaryl is optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy.

In one embodiment R^{4D} is phenyl wherein the phenyl is optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy.

In one embodiment R^{4D} is phenyl wherein the phenyl is optionally substituted with one or more halo.

In one embodiment R^{4D} is 4-fluorophenyl.

In one embodiment R^{5D} is hydrogen, halo, (C₁-C₄)alkyl, (C₁-C₄)haloalkyl, (C₁-C₄)alkoxy, or (C₁-C₄)haloalkoxy.

In one embodiment R^{5D} is hydrogen.

In one embodiment R^{6D} is hydrogen, halo, (C₁-C₄)alkyl, (C₁-C₄)haloalkyl, (C₁-C₄)alkoxy, or (C₁-C₄)haloalkoxy.

In one embodiment R^{6D} is hydrogen.

In one embodiment B″ is (C₃-C₇)carbocyclyl, (C₃-C₇)carbocyclyl-(C₁-C₄)alkyl-, aryl, aryl-(C₁-C₄)alkyl-, heteroaryl, or heteroaryl-(C₁-C₄)alkyl-, wherein any C₃-C₇)carbocyclyl, (C₃—C₇)carbocyclyl-(C₁-C₄)alkyl-, aryl, aryl-(C₁-C₄)alkyl-, heteroaryl, or heteroaryl-(C₁-C₄)alkyl- of B″ is optionally substituted with one or more Z¹ groups.

In one embodiment B″ is (C₃-C₇)carbocyclyl, aryl, aryl-(C₁-C₄)alkyl-, or heteroaryl wherein any (C₃-C₇)carbocyclyl, aryl, aryl-(C₁-C₄)alkyl-, or heteroaryl, of B″ is optionally substituted with one or more Z¹ groups.

In one embodiment B″ is (C₃-C₇)carbocyclyl, phenyl, phenyl-(C₁-C₄)alkyl-, or 5-6 membered heteroaryl wherein any (C₃-C₇)carbocyclyl, phenyl, phenyl-(C₁-C₄)alkyl-, or 5-6 membered heteroaryl of B″ is optionally substituted with one or more Z¹ groups.

In one embodiment B″ is (C₃-C₇)carbocyclyl, phenyl, phenyl-(C₁-C₄)alkyl-, or 5-6 membered heteroaryl wherein any (C₃-C₇)carbocyclyl, phenyl, phenyl-(C₁-C₄)alkyl-, or 5-6 membered heteroaryl of B″ is optionally substituted with one or more Z¹ groups.

In one embodiment B″ is (C₃-C₇)carbocyclyl, phenyl, phenyl-(C₁-C₄)alkyl-, or 6 membered heteroaryl wherein any (C₃-C₇)carbocyclyl, phenyl, phenyl-(C₁-C₄)alkyl-, or 6 membered heteroaryl of B″ is optionally substituted with one or more Z¹ groups.

In one embodiment B″ is (C₃-C₇)carbocyclyl, phenyl, phenyl-(CH₂)—, or pyridinyl wherein any phenyl, phenyl-(CH₂)—, or pyridinyl of B″ is optionally substituted with one or more Z¹ groups.

In one embodiment each Z¹ is independently halo, —OH, or (C₁-C₄)haloalkyl.

In one embodiment B″ is 4-fluorophenyl, cyclopropyl, benzyl, pyrdin-4-yl, 4-hydroxyphenyl, or 4-trifluoromethylphenyl.

In one embodiment R^{1D} is (C₁-C₁₄)alkyl substituted with one or more groups independently selected from —NR^{b2}R^{c2} and wherein the (C₁-C₁₄)alkyl is optionally substituted with one or more (C₃-C₇)carbocyclyl.

In one embodiment R^{1D} is (C₂-C₁₀)alkyl substituted with one or more groups independently selected from —NR^{b2}R^{c2} and wherein the (C₂-C₁₀)alkyl is optionally substituted with one or more (C₃-C₇)carbocyclyl.

In one embodiment R^{1D} is (C₄-C₅)alkyl substituted with two or more groups independently selected from —NR^{b2}R^{c2}.

In one embodiment R^{b2} and R^{c2} are each hydrogen.

In one embodiment R^{1D} is a 4-7 membered monocyclic heterocyclyl-(C₁-C₄)alkyl-, wherein the 4-7 membered monocyclic heterocyclyl-(C₁-C₄)alkyl- is substituted with one or more groups independently selected from the group consisting of Z and —(C₁-C₆)alkyl substituted with one or more Z, wherein each Z is independently selected from the group consisting of —NR^{b3}R^{c3}, —NHNH₂, —C(═NR^{a3})(NR^{b3}R^{c3})_—NR^{a3}C(═NR^{a3})(R^{d3}), and —NR^{a3}C(═NR^{a3})(NR^{b3}R^{c3}) and wherein the 4-7 membered monocyclic heterocyclyl-(C₁-C₄)alkyl- is optionally substituted with one or more (C₁-C₆)alkyl.

In one embodiment R^{1D} is a 4-7 membered monocyclic heterocyclyl-(C₁-C₄)alkyl-, wherein the 4-7 membered monocyclic heterocyclyl-(C₁-C₄)alkyl- is substituted with one or more groups independently selected from the group consisting of Z and (C₁-C₆)alkyl substituted with one or more Z, wherein each Z is independently —NR^{b3}R^{c3} and wherein the 4-7 membered monocyclic heterocyclyl-(C₁-C₄)alkyl- is optionally substituted with one or more (C₁-C₆)alkyl.

In one embodiment R^{1D} is pyrrolidinyl-(C₁-C₄)alkyl-, wherein the pyrrolidinyl-(C₁-C₄)alkyl- is substituted with one or more groups independently selected from the group consisting of Z and —(C₁-C₆)alkyl substituted with one or more Z, wherein each Z is independently —NR^{b3}R^{c3} and wherein is pyrrolidinyl-(C₁-C₄)alkyl- is optionally substituted independently with one or more (C₁-C₆)alkyl.

In one embodiment R^{1D} is pyrrolidinyl-(CH₂)—, wherein the pyrrolidinyl-(CH₂)— is substituted with one or more groups independently selected from the group consisting of Z and —(C₁-C₆)alkyl substituted with one or more Z, wherein each Z is independently —NR^{b3}R^{c3} and wherein the pyrrolidinyl-(CH₂)— is optionally substituted independently with one or more (C₁-C₆)alkyl.

In one embodiment R^{1D} is pyrrolidinyl-(CH₂)—, wherein the pyrrolidinyl-(CH₂)— is substituted on the pyrrolidinyl with —(C₁-C₆)alkyl substituted with one or more —NR^{b3}R^{c3}.

In one embodiment R^{b3} and R³ are each hydrogen.

In one embodiment R^{1D} is:

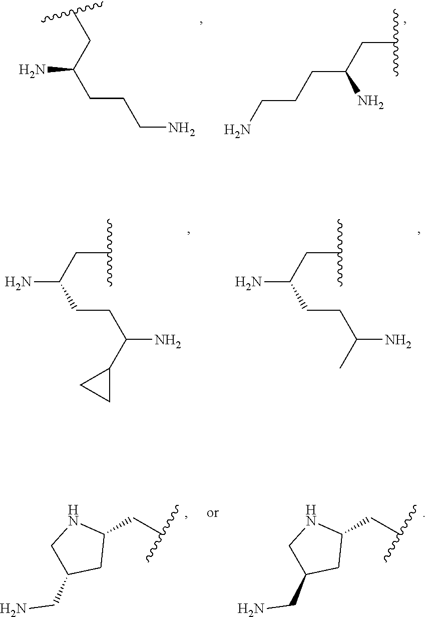

In one embodiment A″ is:

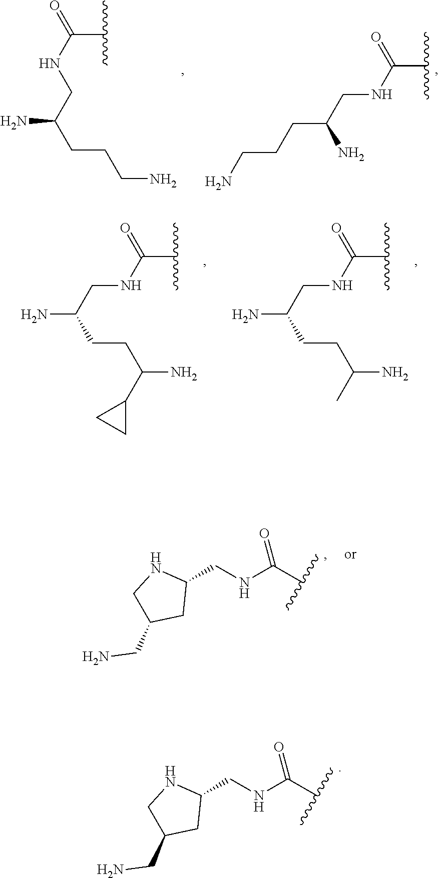 [SMILES: *C(=O)NC[C@H](N)CCCN ; *C(=O)NC[C@@H](N)CCCN ; *C(=O)NC[C@@H](N)CCC(N)C1CC1 ; *C(=O)NC[C@@H](N)CCC(C)N ; *C(=O)NC[C@@H]1C[C@H](CN)CN1 ; *C(=O)NC[C@@H]1C[C@@H](CN)CN1]

In one embodiment, the bacterial efflux pump inhibitor is selected from the group consisting of:

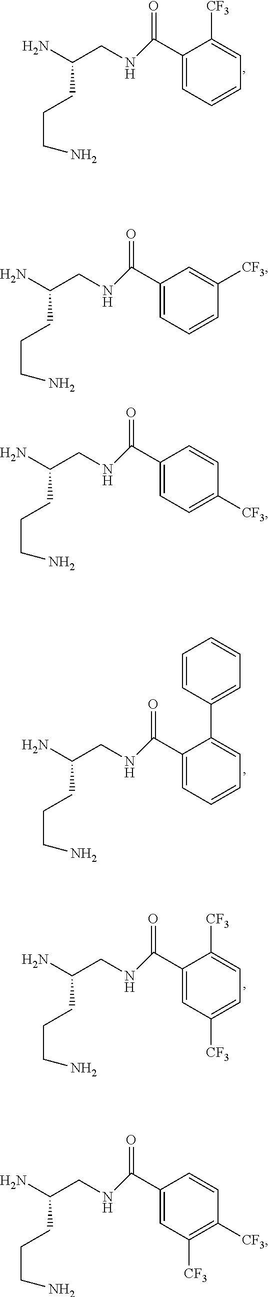 [SMILES: NCCC[C@H](N)CNC(=O)c1ccccc1C(F)(F)F ; NCCC[C@H](N)CNC(=O)c1cccc(C(F)(F)F)c1 ; NCCC[C@H](N)CNC(=O)c1ccc(C(F)(F)F)cc1 ; NCCC[C@H](N)CNC(=O)c1ccccc1-c1ccccc1 ; NCCC[C@H](N)CNC(=O)c1cc(C(F)(F)F)ccc1C(F)(F)F ; NCCC[C@H](N)CNC(=O)c1ccc(C(F)(F)F)c(C(F)(F)F)c1]

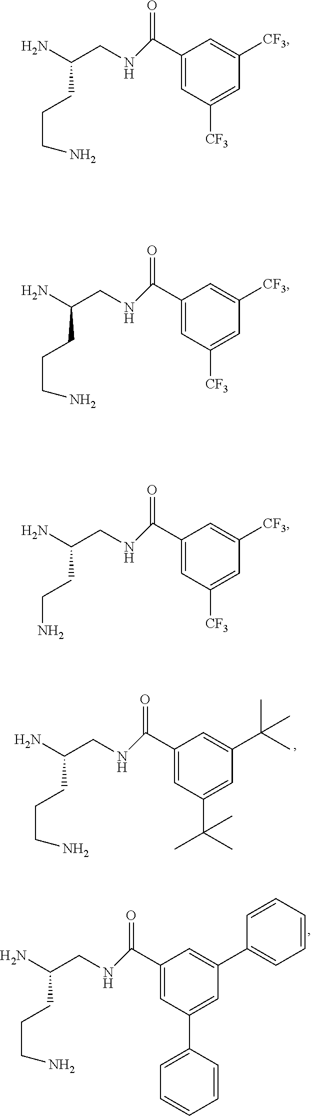 [SMILES: NCCC[C@H](N)CNC(=O)c1cc(C(F)(F)F)cc(C(F)(F)F)c1 ; NCCC[C@@H](N)CNC(=O)c1cc(C(F)(F)F)cc(C(F)(F)F)c1 ; NCC[C@H](N)CNC(=O)c1cc(C(F)(F)F)cc(C(F)(F)F)c1 ; CC(C)(C)c1cc(C(=O)NC[C@@H](N)CCCN)cc(C(C)(C)C)c1 ; NCCC[C@H](N)CNC(=O)c1cc(-c2ccccc2)cc(-c2ccccc2)c1]

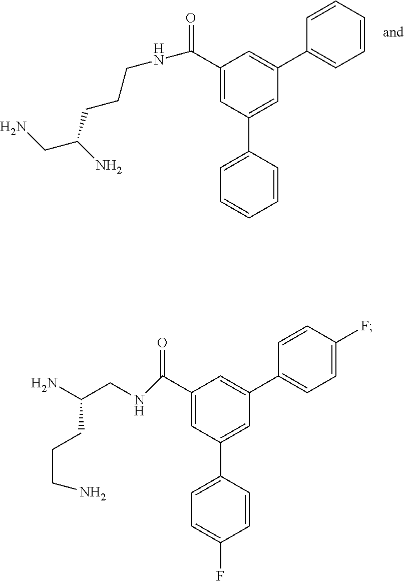 [SMILES: NC[C@@H](N)CCCNC(=O)c1cc(-c2ccccc2)cc(-c2ccccc2)c1 ; NCCC[C@H](N)CNC(=O)c1cc(-c2ccc(F)cc2)cc(-c2ccc(F)cc2)c1]

or a pharmaceutically acceptable salt thereof.

In one embodiment, the bacterial efflux pump inhibitor is selected from the group consisting of:

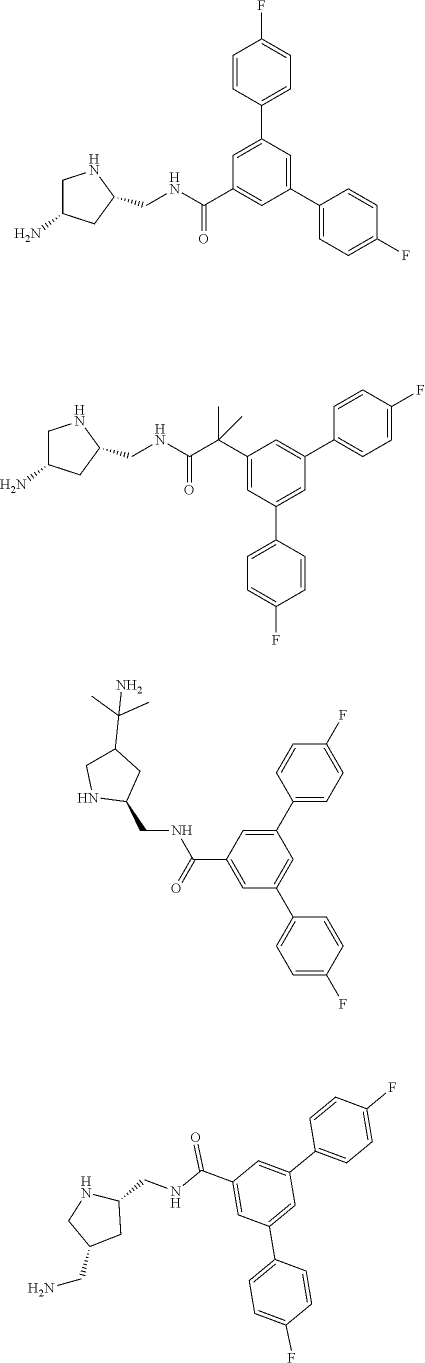 [SMILES: N[C@@H]1CN[C@H](CNC(=O)c2cc(-c3ccc(F)cc3)cc(-c3ccc(F)cc3)c2)C1 ; CC(C)(C(=O)NC[C@@H]1C[C@H](N)CN1)c1cc(-c2ccc(F)cc2)cc(-c2ccc(F)cc2)c1 ; CC(C)(N)C1CN[C@H](CNC(=O)c2cc(-c3ccc(F)cc3)cc(-c3ccc(F)cc3)c2)C1 ; NC[C@@H]1CN[C@H](CNC(=O)c2cc(-c3ccc(F)cc3)cc(-c3ccc(F)cc3)c2)C1]

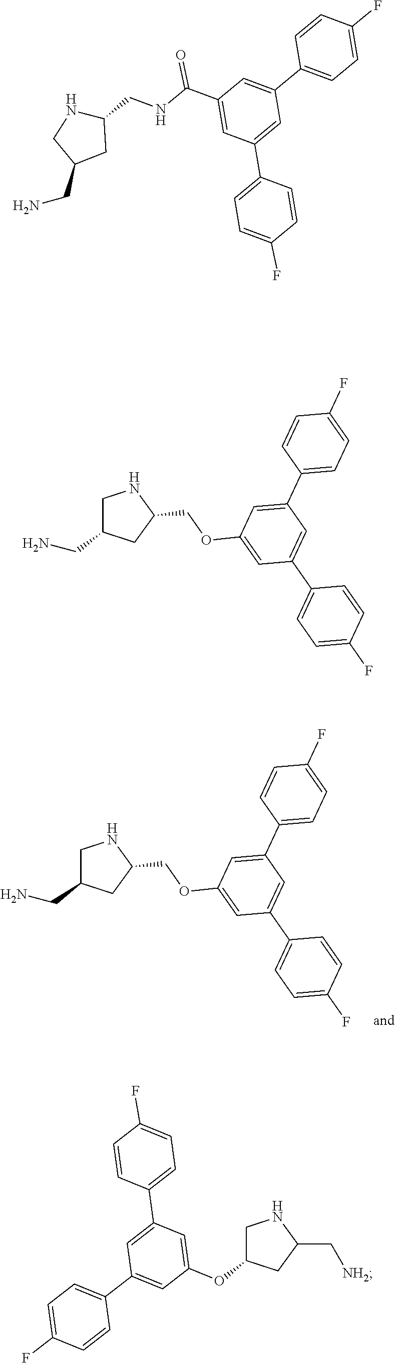 [SMILES: NC[C@H]1CN[C@H](CNC(=O)c2cc(-c3ccc(F)cc3)cc(-c3ccc(F)cc3)c2)C1 ; NC[C@@H]1CN[C@H](COc2cc(-c3ccc(F)cc3)cc(-c3ccc(F)cc3)c2)C1 ; NC[C@H]1CN[C@H](COc2cc(-c3ccc(F)cc3)cc(-c3ccc(F)cc3)c2)C1 ; NCC1C[C@H](Oc2cc(-c3ccc(F)cc3)cc(-c3ccc(F)cc3)c2)CN1]

or a pharmaceutically acceptable salt thereof.

In one embodiment, the bacterial efflux pump inhibitor is selected from the group consisting of:

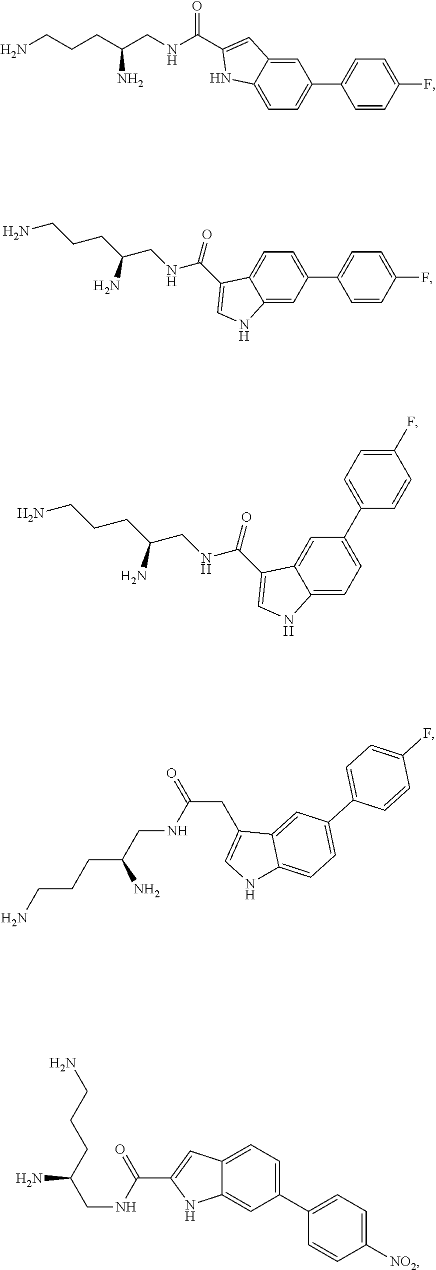 [SMILES: NCCC[C@H](N)CNC(=O)c1cc2cc(-c3ccc(F)cc3)ccc2[nH]1 ; NCCC[C@H](N)CNC(=O)c1c[nH]c2cc(-c3ccc(F)cc3)ccc12 ; NCCC[C@H](N)CNC(=O)c1c[nH]c2ccc(-c3ccc(F)cc3)cc12 ; NCCC[C@H](N)CNC(=O)Cc1c[nH]c2ccc(-c3ccc(F)cc3)cc12 ; NCCC[C@H](N)CNC(=O)c1cc2ccc(-c3ccc([N+](=O)[O-])cc3)cc2[nH]1]

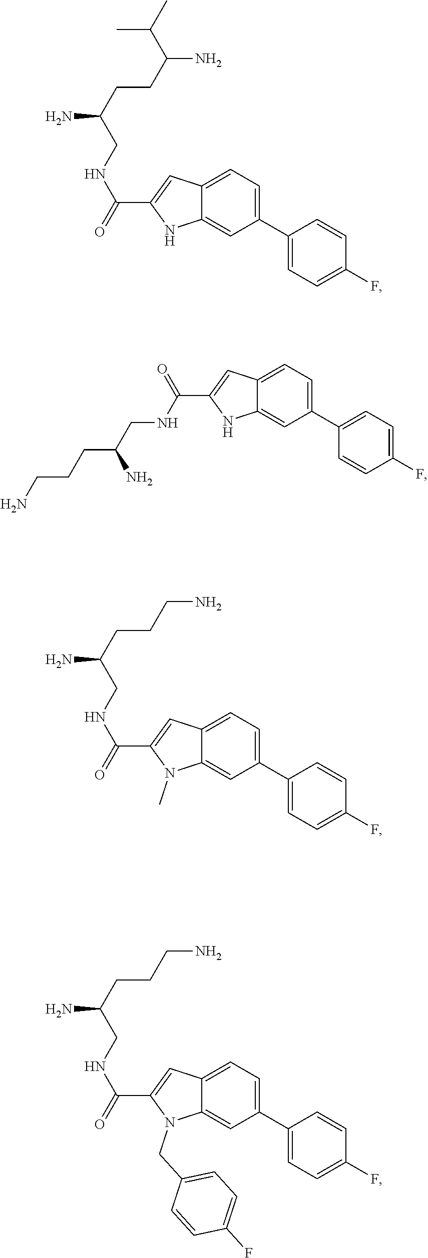 [SMILES: CC(C)C(N)CC[C@H](N)CNC(=O)c1cc2ccc(-c3ccc(F)cc3)cc2[nH]1 ; NCCC[C@H](N)CNC(=O)c1cc2ccc(-c3ccc(F)cc3)cc2[nH]1 ; Cn1c(C(=O)NC[C@@H](N)CCCN)cc2ccc(-c3ccc(F)cc3)cc21 ; NCCC[C@H](N)CNC(=O)c1cc2ccc(-c3ccc(F)cc3)cc2n1Cc1ccc(F)cc1]

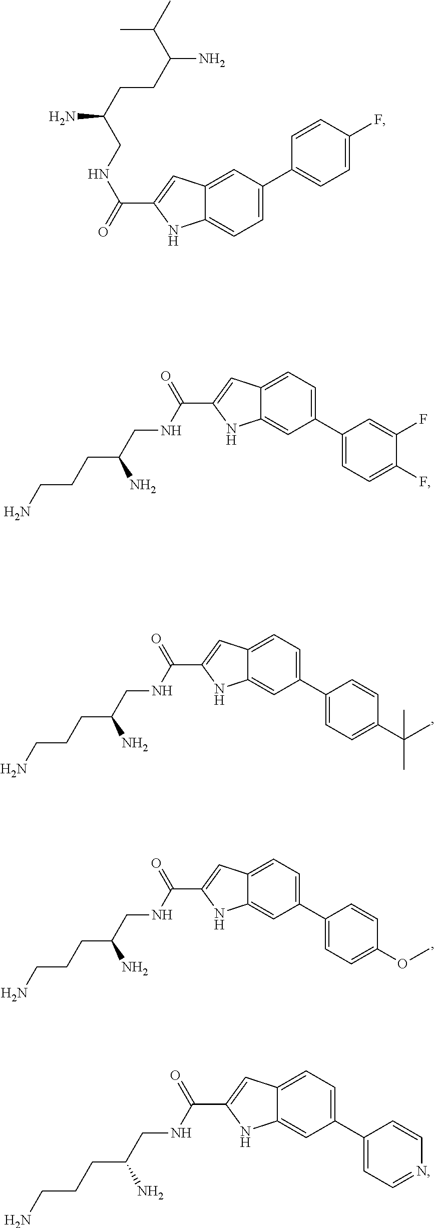 [SMILES: CC(C)C(N)CC[C@H](N)CNC(=O)c1cc2cc(-c3ccc(F)cc3)ccc2[nH]1 ; NCCC[C@H](N)CNC(=O)c1cc2ccc(-c3ccc(F)c(F)c3)cc2[nH]1 ; CC(C)(C)c1ccc(-c2ccc3cc(C(=O)NC[C@@H](N)CCCN)[nH]c3c2)cc1 ; COc1ccc(-c2ccc3cc(C(=O)NC[C@@H](N)CCCN)[nH]c3c2)cc1 ; NCCC[C@@H](N)CNC(=O)c1cc2ccc(-c3ccncc3)cc2[nH]1]

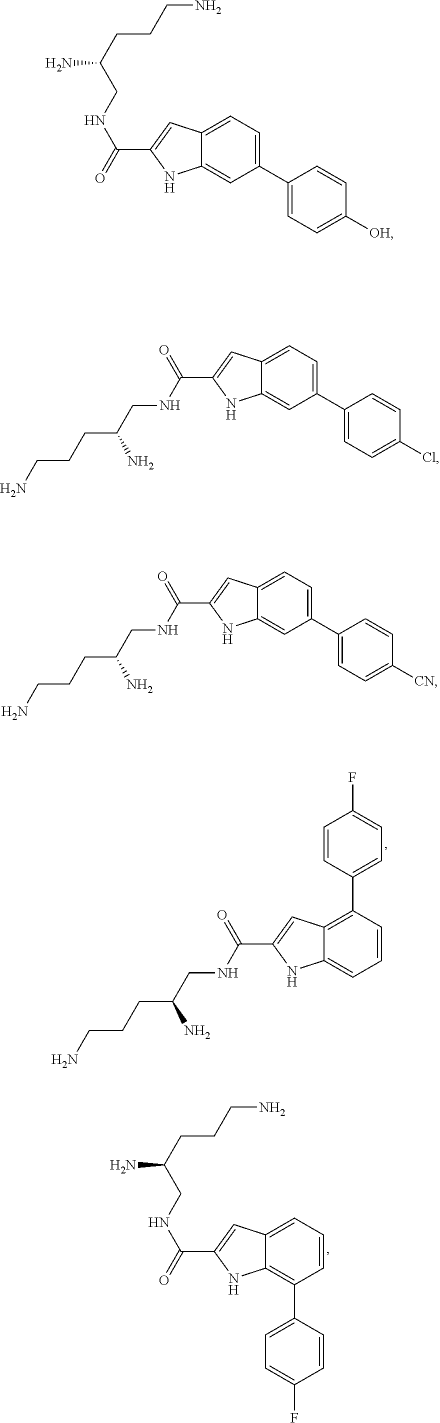 [SMILES: NCCC[C@@H](N)CNC(=O)c1cc2ccc(-c3ccc(O)cc3)cc2[nH]1 ; NCCC[C@@H](N)CNC(=O)c1cc2ccc(-c3ccc(Cl)cc3)cc2[nH]1 ; N#Cc1ccc(-c2ccc3cc(C(=O)NC[C@H](N)CCCN)[nH]c3c2)cc1 ; NCCC[C@H](N)CNC(=O)c1cc2c(-c3ccc(F)cc3)cccc2[nH]1 ; NCCC[C@H](N)CNC(=O)c1cc2cccc(-c3ccc(F)cc3)c2[nH]1]

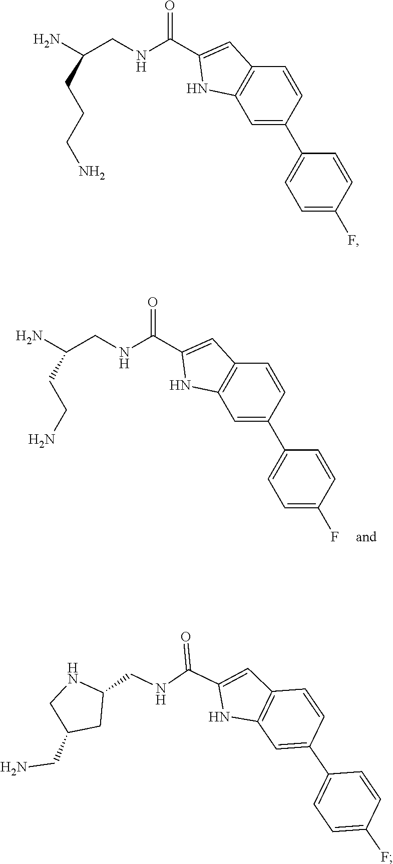 [SMILES: NCCC[C@@H](N)CNC(=O)c1cc2ccc(-c3ccc(F)cc3)cc2[nH]1 ; NCC[C@H](N)CNC(=O)c1cc2ccc(-c3ccc(F)cc3)cc2[nH]1 ; "and" ; NC[C@@H]1CN[C@H](CNC(=O)c2cc3ccc(-c4ccc(F)cc4)cc3[nH]2)C1]

or a pharmaceutically acceptable salt thereof.

In one embodiment, the bacterial efflux pump inhibitor is selected from the group consisting of:

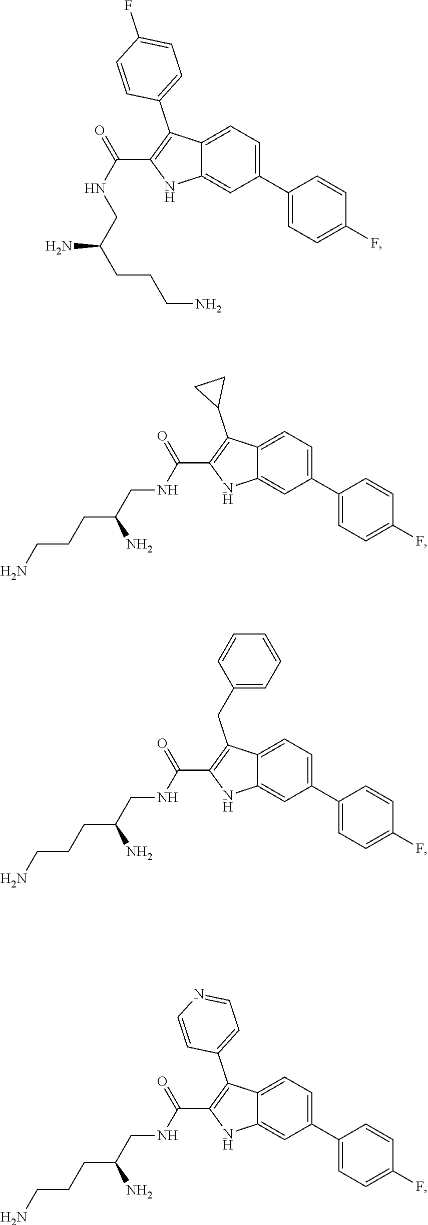 [SMILES: NCCC[C@@H](N)CNC(=O)c1[nH]c2cc(-c3ccc(F)cc3)ccc2c1-c1ccc(F)cc1 ; NCCC[C@H](N)CNC(=O)c1[nH]c2cc(-c3ccc(F)cc3)ccc2c1C1CC1 ; NCCC[C@H](N)CNC(=O)c1[nH]c2cc(-c3ccc(F)cc3)ccc2c1Cc1ccccc1 ; NCCC[C@H](N)CNC(=O)c1[nH]c2cc(-c3ccc(F)cc3)ccc2c1-c1ccncc1]

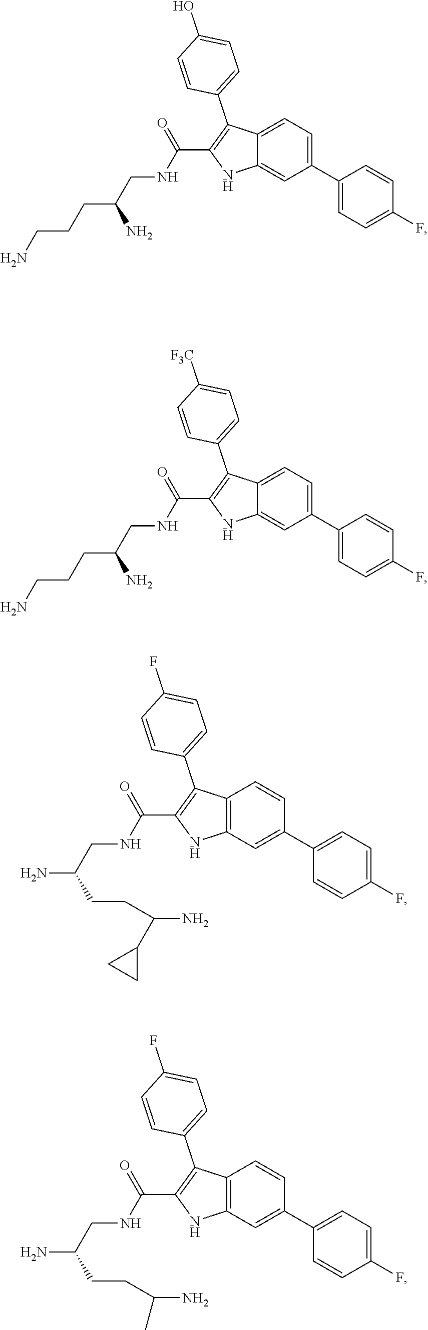 [SMILES: NCCC[C@H](N)CNC(=O)c1[nH]c2cc(-c3ccc(F)cc3)ccc2c1-c1ccc(O)cc1 ; NCCC[C@H](N)CNC(=O)c1[nH]c2cc(-c3ccc(F)cc3)ccc2c1-c1ccc(C(F)(F)F)cc1 ; NC(CC[C@H](N)CNC(=O)c1[nH]c2cc(-c3ccc(F)cc3)ccc2c1-c1ccc(F)cc1)C1CC1 ; CC(N)CC[C@H](N)CNC(=O)c1[nH]c2cc(-c3ccc(F)cc3)ccc2c1-c1ccc(F)cc1]

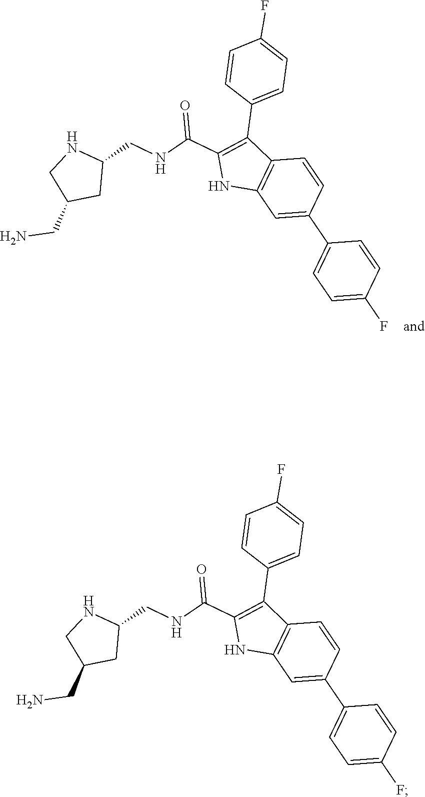 [SMILES: NC[C@@H]1CN[C@H](CNC(=O)c2[nH]c3cc(-c4ccc(F)cc4)ccc3c2-c2ccc(F)cc2)C1 ; "and" ; NC[C@H]1CN[C@H](CNC(=O)c2[nH]c3cc(-c4ccc(F)cc4)ccc3c2-c2ccc(F)cc2)C1]

or a pharmaceutically acceptable salt thereof.

In one embodiment, the bacterial efflux pump inhibitor is

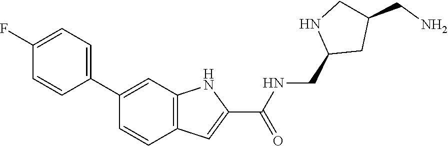 [SMILES: NC[C@@H]1CN[C@H](CNC(=O)c2cc3ccc(-c4ccc(F)cc4)cc3[nH]2)C1]

or a pharmaceutically acceptable salt thereof.

In one embodiment, the bacterial efflux pump inhibitor is selected from the group consisting of:

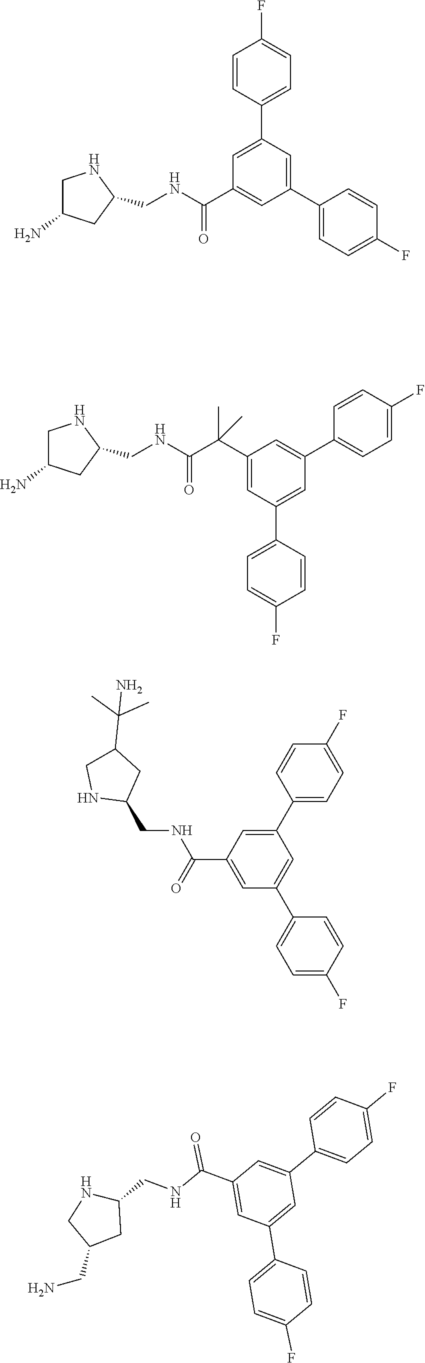 [SMILES: N[C@@H]1CN[C@H](CNC(=O)c2cc(-c3ccc(F)cc3)cc(-c3ccc(F)cc3)c2)C1 ; CC(C)(C(=O)NC[C@@H]1C[C@H](N)CN1)c1cc(-c2ccc(F)cc2)cc(-c2ccc(F)cc2)c1 ; CC(C)(N)C1CN[C@H](CNC(=O)c2cc(-c3ccc(F)cc3)cc(-c3ccc(F)cc3)c2)C1 ; NC[C@@H]1CN[C@H](CNC(=O)c2cc(-c3ccc(F)cc3)cc(-c3ccc(F)cc3)c2)C1]

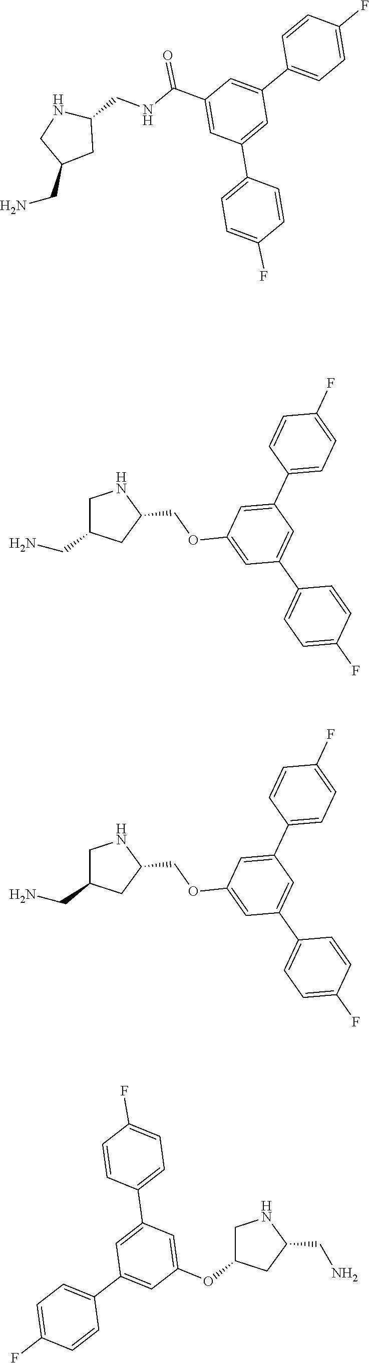 [SMILES: NC[C@H]1CN[C@H](CNC(=O)c2cc(-c3ccc(F)cc3)cc(-c3ccc(F)cc3)c2)C1 ; NC[C@@H]1CN[C@H](COc2cc(-c3ccc(F)cc3)cc(-c3ccc(F)cc3)c2)C1 ; NC[C@H]1CN[C@H](COc2cc(-c3ccc(F)cc3)cc(-c3ccc(F)cc3)c2)C1 ; NC[C@@H]1C[C@H](Oc2cc(-c3ccc(F)cc3)cc(-c3ccc(F)cc3)c2)CN1]

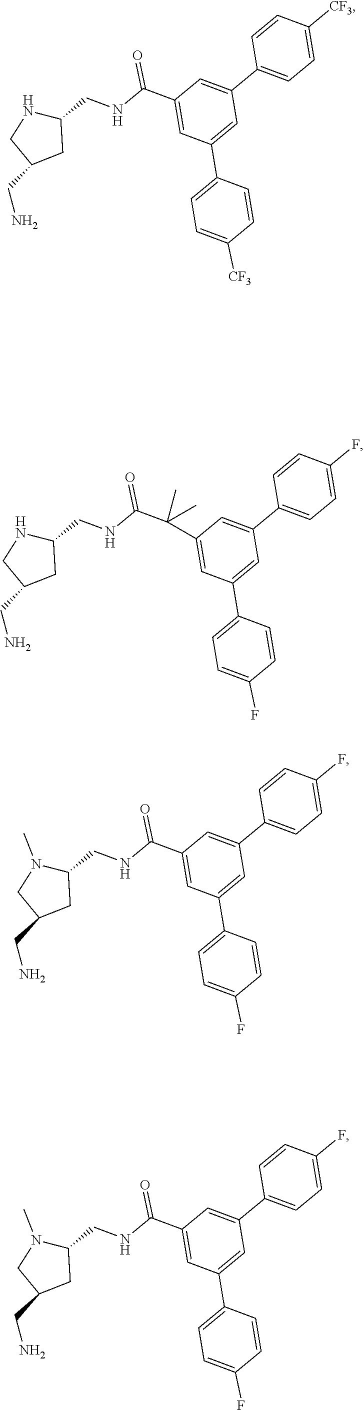 [SMILES: NC[C@@H]1CN[C@H](CNC(=O)c2cc(-c3ccc(C(F)(F)F)cc3)cc(-c3ccc(C(F)(F)F)cc3)c2)C1 ; CC(C)(C(=O)NC[C@@H]1C[C@H](CN)CN1)c1cc(-c2ccc(F)cc2)cc(-c2ccc(F)cc2)c1 ; CN1C[C@H](CN)C[C@H]1CNC(=O)c1cc(-c2ccc(F)cc2)cc(-c2ccc(F)cc2)c1 ; CN1C[C@H](CN)C[C@H]1CNC(=O)c1cc(-c2ccc(F)cc2)cc(-c2ccc(F)cc2)c1]

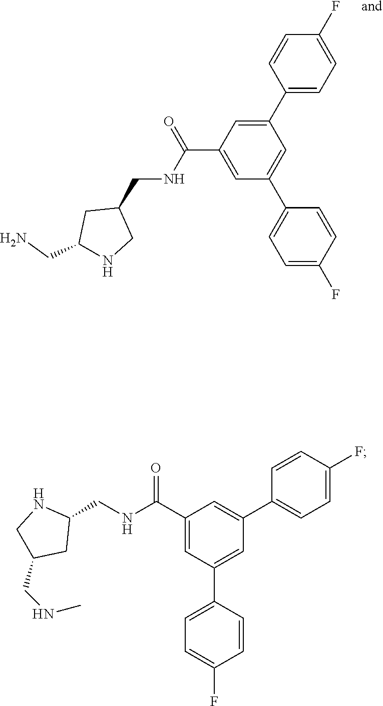 [SMILES: NC[C@@H]1C[C@@H](CNC(=O)c2cc(-c3ccc(F)cc3)cc(-c3ccc(F)cc3)c2)CN1 ; "and" ; CNC[C@@H]1CN[C@H](CNC(=O)c2cc(-c3ccc(F)cc3)cc(-c3ccc(F)cc3)c2)C1]

or a pharmaceutically acceptable salt thereof.

In one embodiment, the bacterial efflux pump inhibitor is selected from the group consisting of:

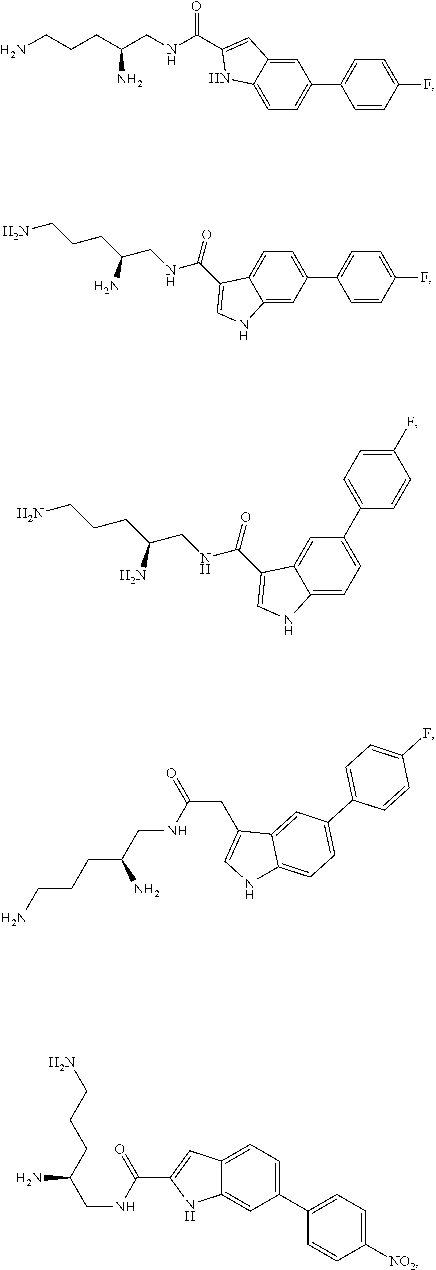 [SMILES: NCCC[C@H](N)CNC(=O)c1cc2cc(-c3ccc(F)cc3)ccc2[nH]1 ; NCCC[C@H](N)CNC(=O)c1c[nH]c2cc(-c3ccc(F)cc3)ccc12 ; NCCC[C@H](N)CNC(=O)c1c[nH]c2ccc(-c3ccc(F)cc3)cc12 ; NCCC[C@H](N)CNC(=O)Cc1c[nH]c2ccc(-c3ccc(F)cc3)cc12 ; NCCC[C@H](N)CNC(=O)c1cc2ccc(-c3ccc([N+](=O)[O-])cc3)cc2[nH]1]

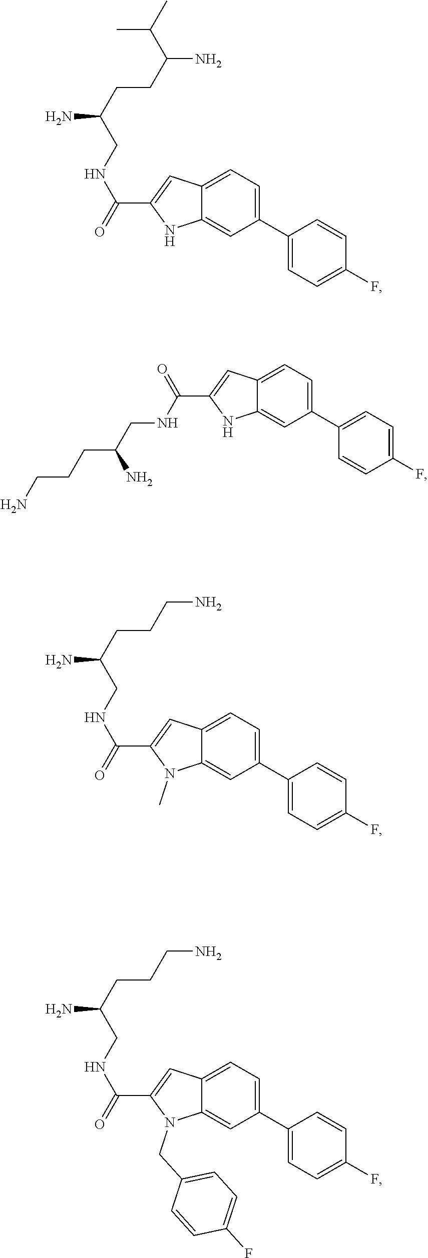 [SMILES: CC(C)C(N)CC[C@H](N)CNC(=O)c1cc2ccc(-c3ccc(F)cc3)cc2[nH]1 ; NCCC[C@H](N)CNC(=O)c1cc2ccc(-c3ccc(F)cc3)cc2[nH]1 ; Cn1c(C(=O)NC[C@@H](N)CCCN)cc2ccc(-c3ccc(F)cc3)cc21 ; NCCC[C@H](N)CNC(=O)c1cc2ccc(-c3ccc(F)cc3)cc2n1Cc1ccc(F)cc1]

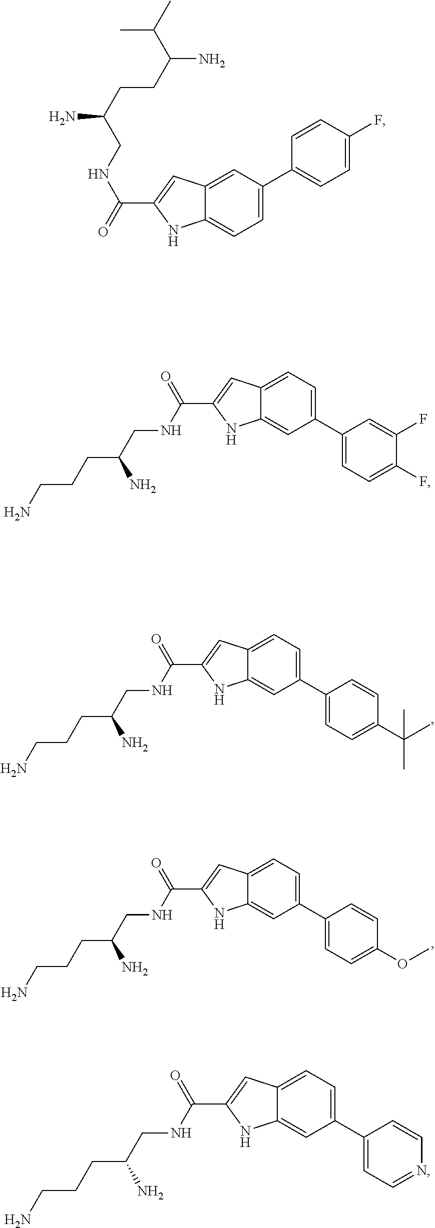 [SMILES: CC(C)C(N)CC[C@H](N)CNC(=O)c1cc2cc(-c3ccc(F)cc3)ccc2[nH]1 ; NCCC[C@H](N)CNC(=O)c1cc2ccc(-c3ccc(F)c(F)c3)cc2[nH]1 ; CC(C)(C)c1ccc(-c2ccc3cc(C(=O)NC[C@@H](N)CCCN)[nH]c3c2)cc1 ; COc1ccc(-c2ccc3cc(C(=O)NC[C@@H](N)CCCN)[nH]c3c2)cc1 ; NCCC[C@@H](N)CNC(=O)c1cc2ccc(-c3ccncc3)cc2[nH]1]

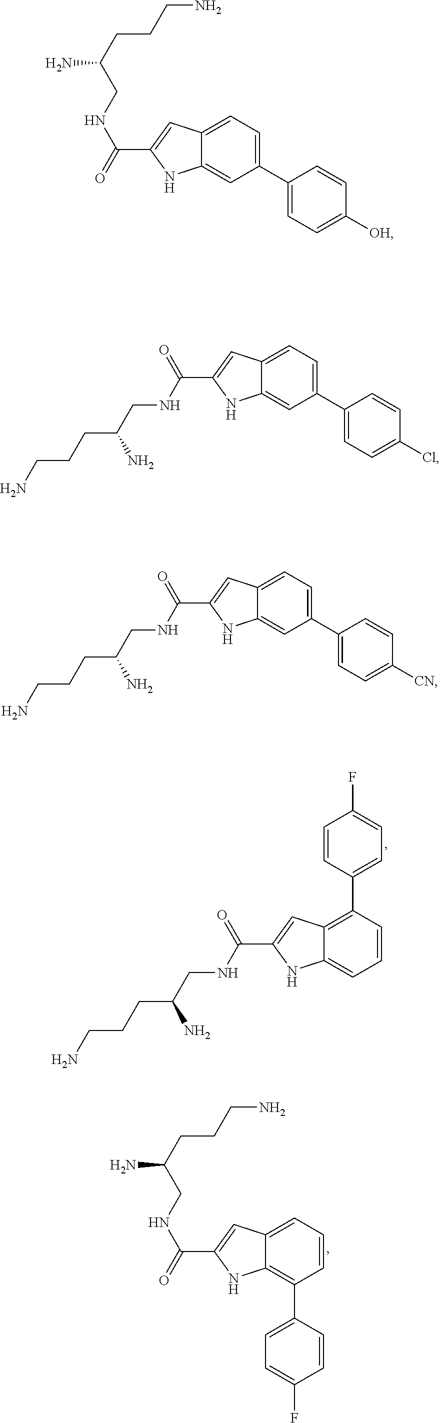 [SMILES: NCCC[C@@H](N)CNC(=O)c1cc2ccc(-c3ccc(O)cc3)cc2[nH]1 ; NCCC[C@@H](N)CNC(=O)c1cc2ccc(-c3ccc(Cl)cc3)cc2[nH]1 ; N#Cc1ccc(-c2ccc3cc(C(=O)NC[C@H](N)CCCN)[nH]c3c2)cc1 ; NCCC[C@H](N)CNC(=O)c1cc2c(-c3ccc(F)cc3)cccc2[nH]1 ; NCCC[C@H](N)CNC(=O)c1cc2cccc(-c3ccc(F)cc3)c2[nH]1]

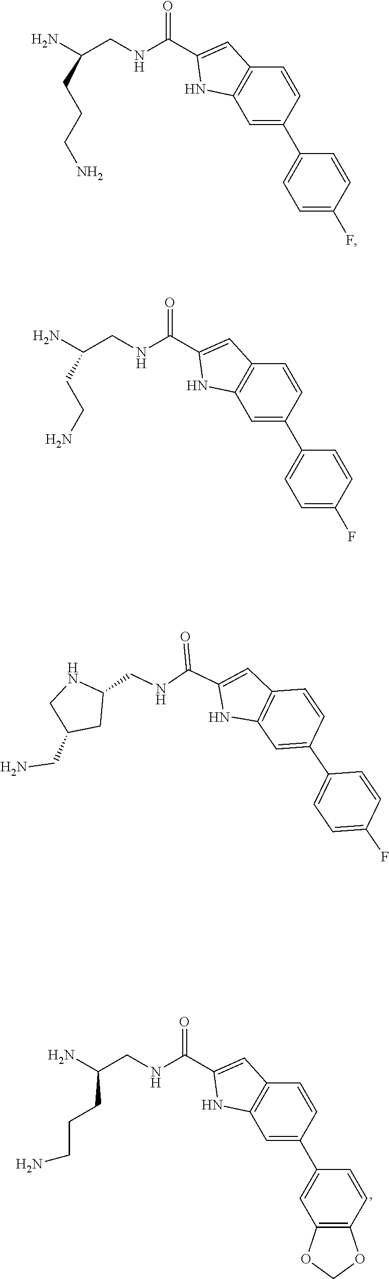 [SMILES: NCCC[C@@H](N)CNC(=O)c1cc2ccc(-c3ccc(F)cc3)cc2[nH]1 ; NCC[C@H](N)CNC(=O)c1cc2ccc(-c3ccc(F)cc3)cc2[nH]1 ; NC[C@@H]1CN[C@H](CNC(=O)c2cc3ccc(-c4ccc(F)cc4)cc3[nH]2)C1 ; NCCC[C@@H](N)CNC(=O)c1cc2ccc(-c3ccc4c(c3)OCO4)cc2[nH]1]

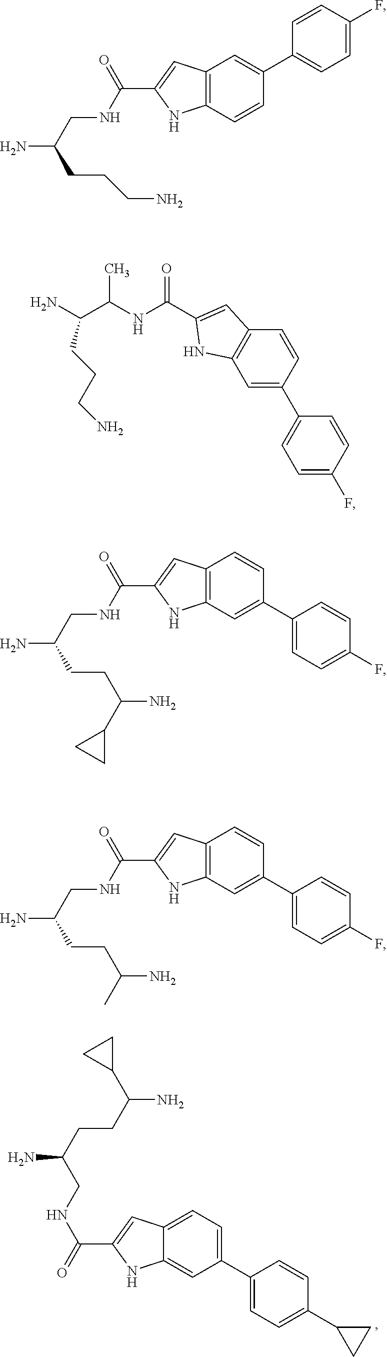

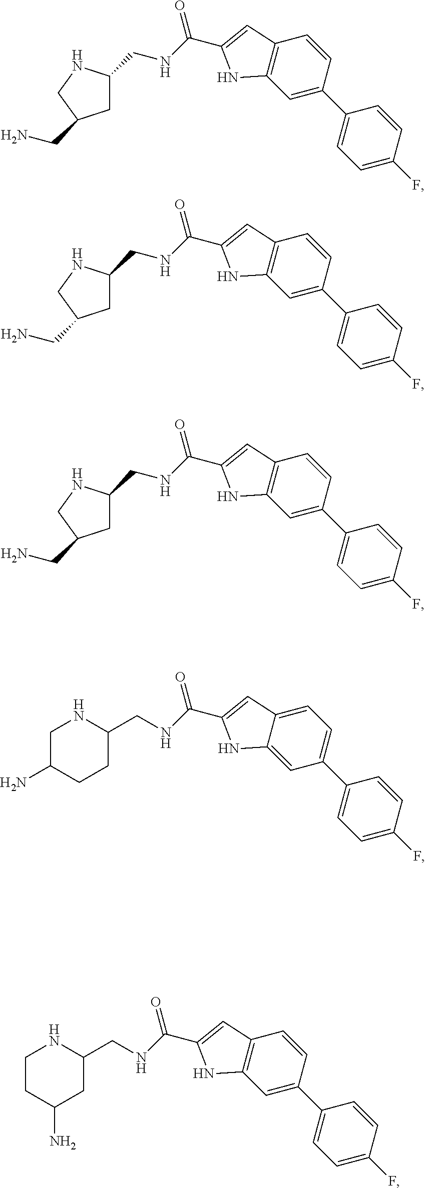 [SMILES: NC[C@H]1CN[C@H](CNC(=O)c2cc3ccc(-c4ccc(F)cc4)cc3[nH]2)C1 ; NC[C@@H]1CN[C@@H](CNC(=O)c2cc3ccc(-c4ccc(F)cc4)cc3[nH]2)C1 ; NC[C@H]1CN[C@@H](CNC(=O)c2cc3ccc(-c4ccc(F)cc4)cc3[nH]2)C1 ; NC1CCC(CNC(=O)c2cc3ccc(-c4ccc(F)cc4)cc3[nH]2)NC1 ; NC1CCNC(CNC(=O)c2cc3ccc(-c4ccc(F)cc4)cc3[nH]2)C1]

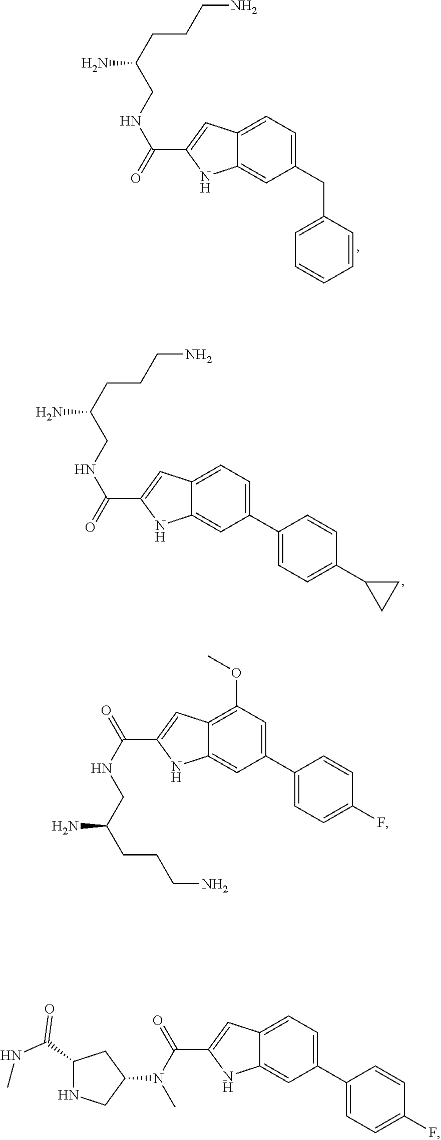 [SMILES: NCCC[C@@H](N)CNC(=O)c1cc2ccc(Cc3ccccc3)cc2[nH]1 ; NCCC[C@@H](N)CNC(=O)c1cc2ccc(-c3ccc(C4CC4)cc3)cc2[nH]1 ; COc1cc(-c2ccc(F)cc2)cc2[nH]c(C(=O)NC[C@H](N)CCCN)cc12 ; CNC(=O)[C@@H]1C[C@H](N(C)C(=O)c2cc3ccc(-c4ccc(F)cc4)cc3[nH]2)CN1]

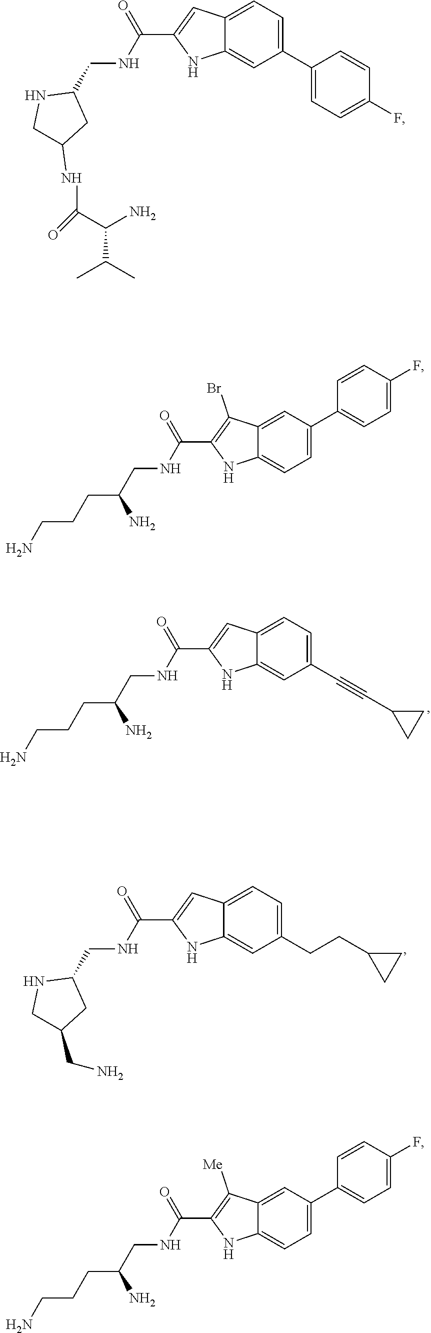

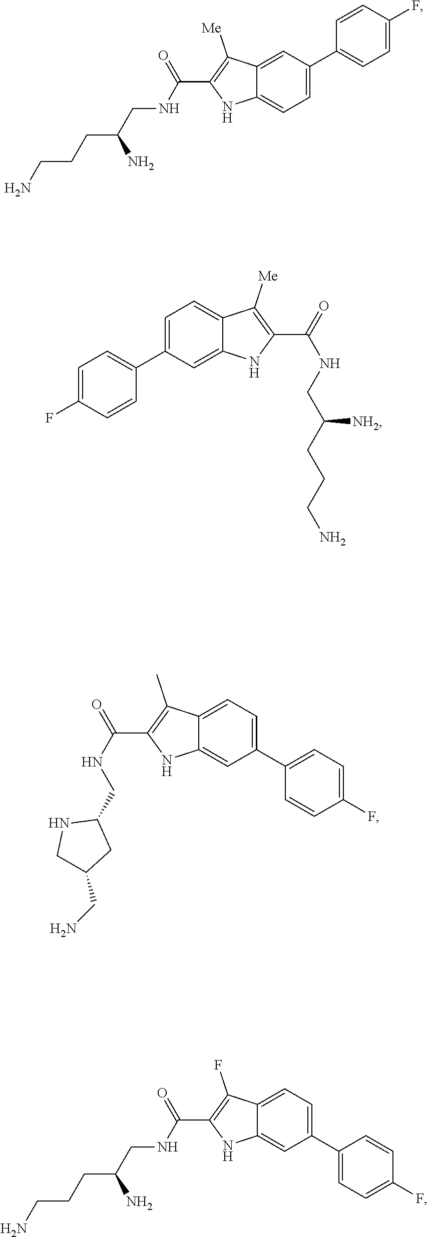 [SMILES: Cc1c(C(=O)NC[C@@H](N)CCCN)[nH]c2ccc(-c3ccc(F)cc3)cc12 ; Cc1c(C(=O)NC[C@@H](N)CCCN)[nH]c2cc(-c3ccc(F)cc3)ccc12 ; Cc1c(C(=O)NC[C@@H]2C[C@H](CN)CN2)[nH]c2cc(-c3ccc(F)cc3)ccc12 ; NCCC[C@H](N)CNC(=O)c1[nH]c2cc(-c3ccc(F)cc3)ccc2c1F]

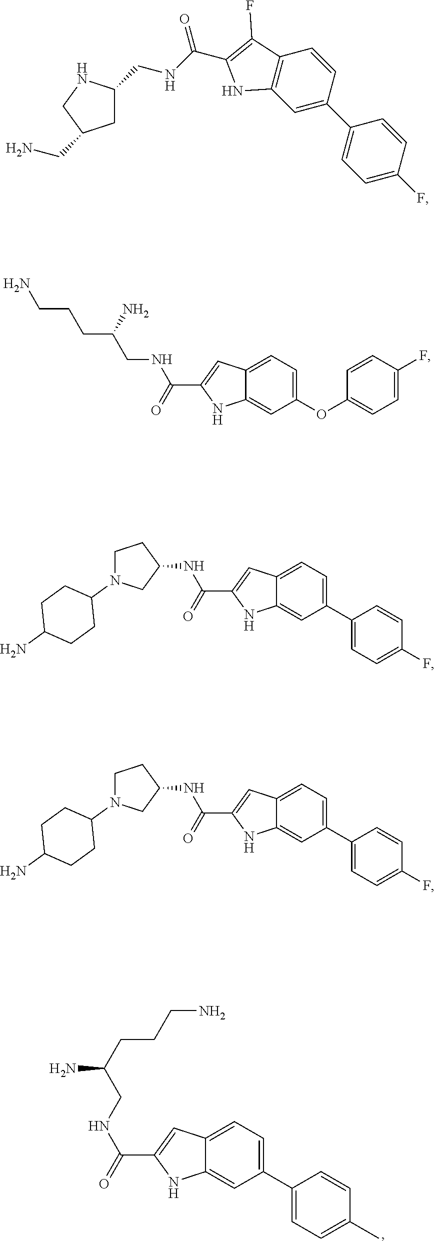 [SMILES: NC[C@@H]1CN[C@H](CNC(=O)c2[nH]c3cc(-c4ccc(F)cc4)ccc3c2F)C1 ; NCCC[C@H](N)CNC(=O)c1cc2ccc(Oc3ccc(F)cc3)cc2[nH]1 ; NC1CCC(N2CC[C@H](NC(=O)c3cc4ccc(-c5ccc(F)cc5)cc4[nH]3)C2)CC1 ; NC1CCC(N2CC[C@H](NC(=O)c3cc4ccc(-c5ccc(F)cc5)cc4[nH]3)C2)CC1 ; Cc1ccc(-c2ccc3cc(C(=O)NC[C@@H](N)CCCN)[nH]c3c2)cc1]

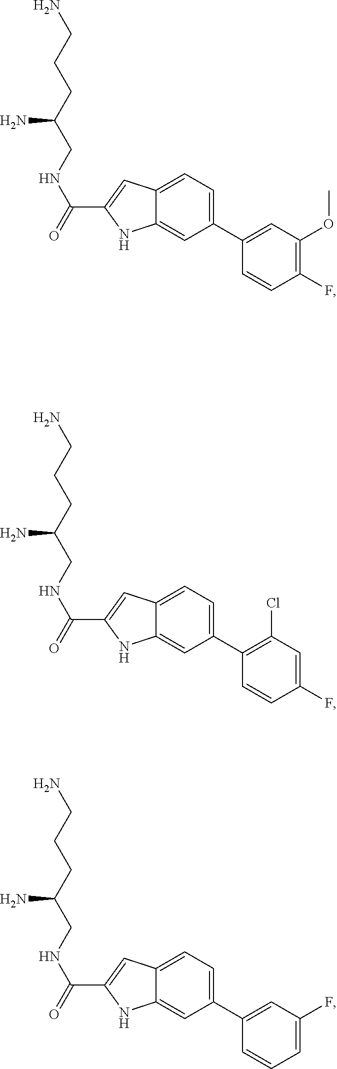 [SMILES: COc1cc(-c2ccc3cc(C(=O)NC[C@@H](N)CCCN)[nH]c3c2)ccc1F ; NCCC[C@H](N)CNC(=O)c1cc2ccc(-c3ccc(F)cc3Cl)cc2[nH]1 ; NCCC[C@H](N)CNC(=O)c1cc2ccc(-c3cccc(F)c3)cc2[nH]1]

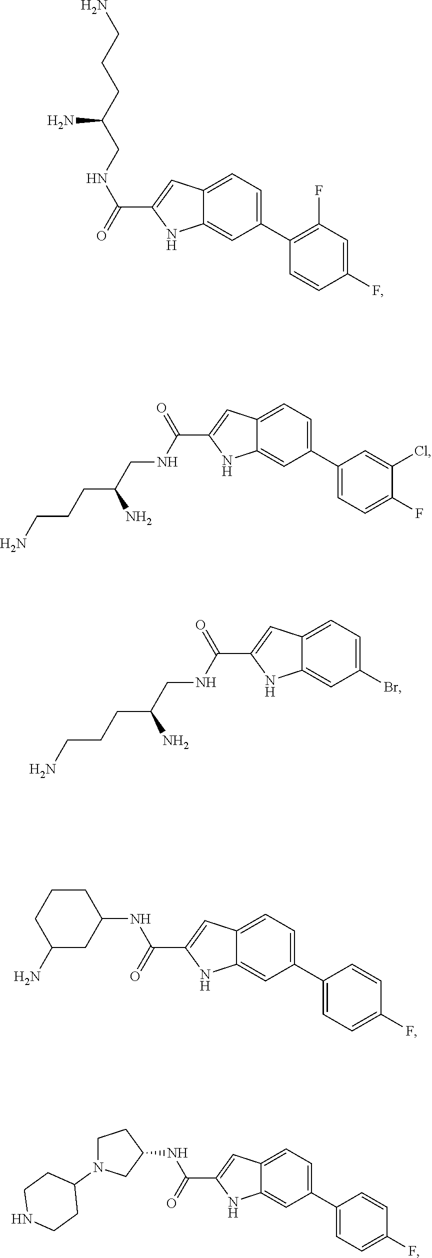 [SMILES: NCCC[C@H](N)CNC(=O)c1cc2ccc(-c3ccc(F)cc3F)cc2[nH]1 ; NCCC[C@H](N)CNC(=O)c1cc2ccc(-c3ccc(F)c(Cl)c3)cc2[nH]1 ; NCCC[C@H](N)CNC(=O)c1cc2ccc(Br)cc2[nH]1 ; NC1CCCC(NC(=O)c2cc3ccc(-c4ccc(F)cc4)cc3[nH]2)C1 ; O=C(N[C@H]1CCN(C2CCNCC2)C1)c1cc2ccc(-c3ccc(F)cc3)cc2[nH]1]

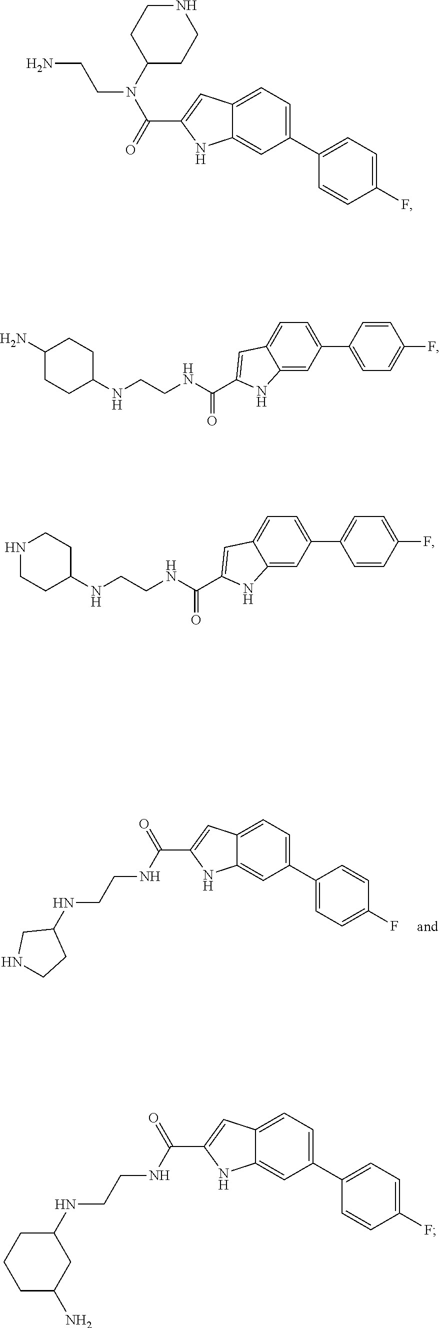 [SMILES: NCCN(C(=O)c1cc2ccc(-c3ccc(F)cc3)cc2[nH]1)C1CCNCC1 ; NC1CCC(NCCNC(=O)c2cc3ccc(-c4ccc(F)cc4)cc3[nH]2)CC1 ; O=C(NCCNC1CCNCC1)c1cc2ccc(-c3ccc(F)cc3)cc2[nH]1 ; O=C(NCCNC1CCNC1)c1cc2ccc(-c3ccc(F)cc3)cc2[nH]1 ; "and" ; NC1CCCC(NCCNC(=O)c2cc3ccc(-c4ccc(F)cc4)cc3[nH]2)C1]

or a pharmaceutically acceptable salt thereof.

In one embodiment, the bacterial efflux pump inhibitor is selected from the group consisting of:

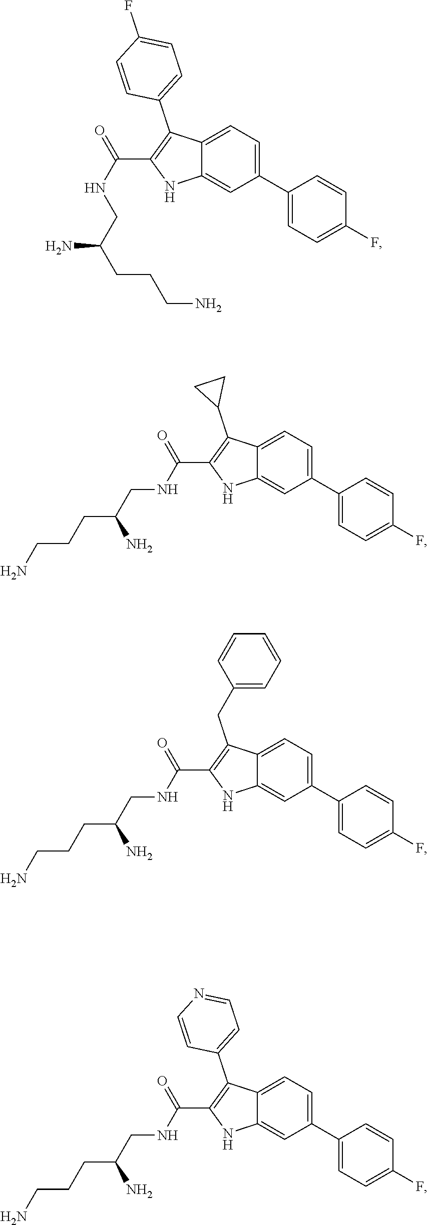 [SMILES: NCCC[C@@H](N)CNC(=O)c1[nH]c2cc(-c3ccc(F)cc3)ccc2c1-c1ccc(F)cc1 ; NCCC[C@H](N)CNC(=O)c1[nH]c2cc(-c3ccc(F)cc3)ccc2c1C1CC1 ; NCCC[C@H](N)CNC(=O)c1[nH]c2cc(-c3ccc(F)cc3)ccc2c1Cc1ccccc1 ; NCCC[C@H](N)CNC(=O)c1[nH]c2cc(-c3ccc(F)cc3)ccc2c1-c1ccncc1]

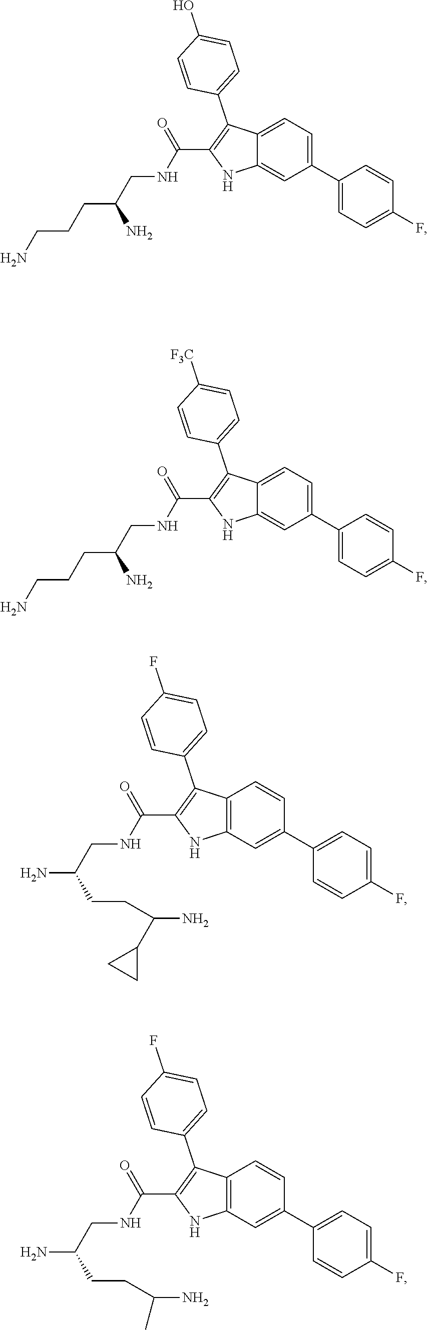 [SMILES: NCCC[C@H](N)CNC(=O)c1[nH]c2cc(-c3ccc(F)cc3)ccc2c1-c1ccc(O)cc1 ; NCCC[C@H](N)CNC(=O)c1[nH]c2cc(-c3ccc(F)cc3)ccc2c1-c1ccc(C(F)(F)F)cc1 ; NC(CC[C@H](N)CNC(=O)c1[nH]c2cc(-c3ccc(F)cc3)ccc2c1-c1ccc(F)cc1)C1CC1 ; CC(N)CC[C@H](N)CNC(=O)c1[nH]c2cc(-c3ccc(F)cc3)ccc2c1-c1ccc(F)cc1]

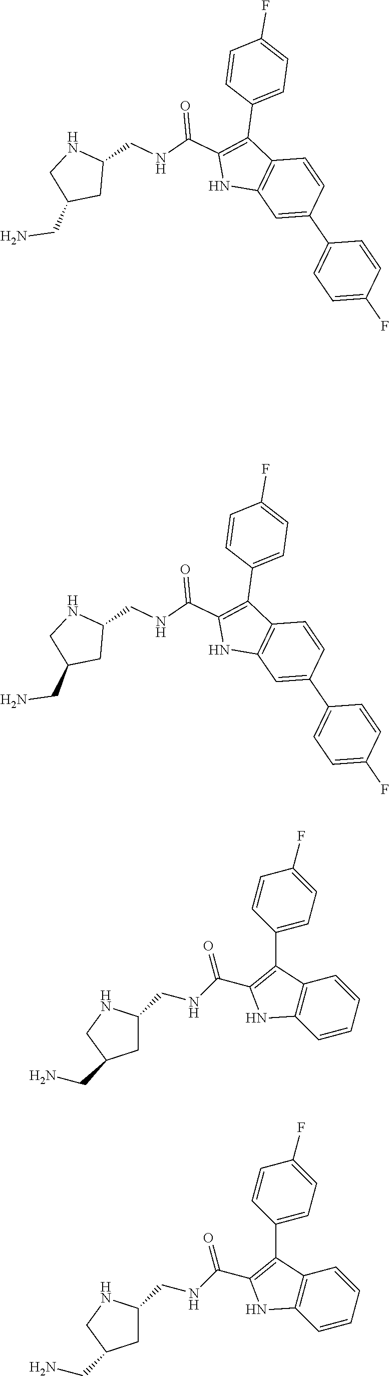 [SMILES: NC[C@@H]1CN[C@H](CNC(=O)c2[nH]c3cc(-c4ccc(F)cc4)ccc3c2-c2ccc(F)cc2)C1 ; NC[C@H]1CN[C@H](CNC(=O)c2[nH]c3cc(-c4ccc(F)cc4)ccc3c2-c2ccc(F)cc2)C1 ; NC[C@H]1CN[C@H](CNC(=O)c2[nH]c3ccccc3c2-c2ccc(F)cc2)C1 ; NC[C@@H]1CN[C@H](CNC(=O)c2[nH]c3ccccc3c2-c2ccc(F)cc2)C1]

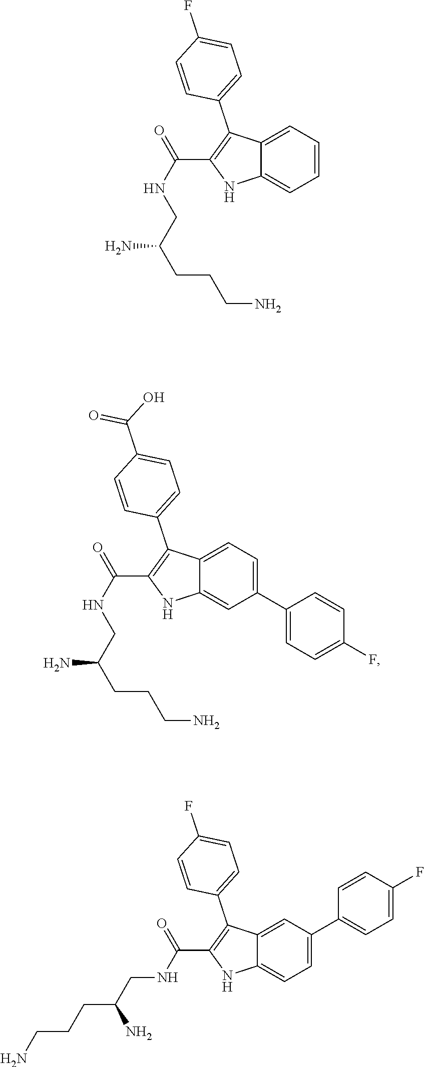 [SMILES: NCCC[C@H](N)CNC(=O)c1[nH]c2ccccc2c1-c1ccc(F)cc1 ; NCCC[C@@H](N)CNC(=O)c1[nH]c2cc(-c3ccc(F)cc3)ccc2c1-c1ccc(C(=O)O)cc1 ; NCCC[C@H](N)CNC(=O)c1[nH]c2ccc(-c3ccc(F)cc3)cc2c1-c1ccc(F)cc1]

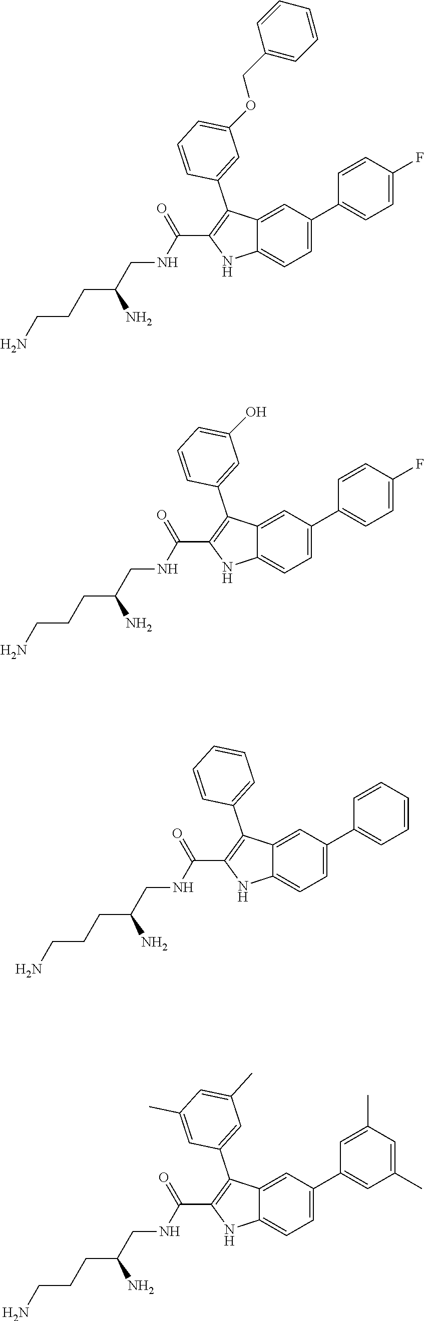 [SMILES: NCCC[C@H](N)CNC(=O)c1[nH]c2ccc(-c3ccc(F)cc3)cc2c1-c1cccc(OCc2ccccc2)c1 ; NCCC[C@H](N)CNC(=O)c1[nH]c2ccc(-c3ccc(F)cc3)cc2c1-c1cccc(O)c1 ; NCCC[C@H](N)CNC(=O)c1[nH]c2ccc(-c3ccccc3)cc2c1-c1ccccc1 ; Cc1cc(C)cc(-c2ccc3[nH]c(C(=O)NC[C@@H](N)CCCN)c(-c4cc(C)cc(C)c4)c3c2)c1]

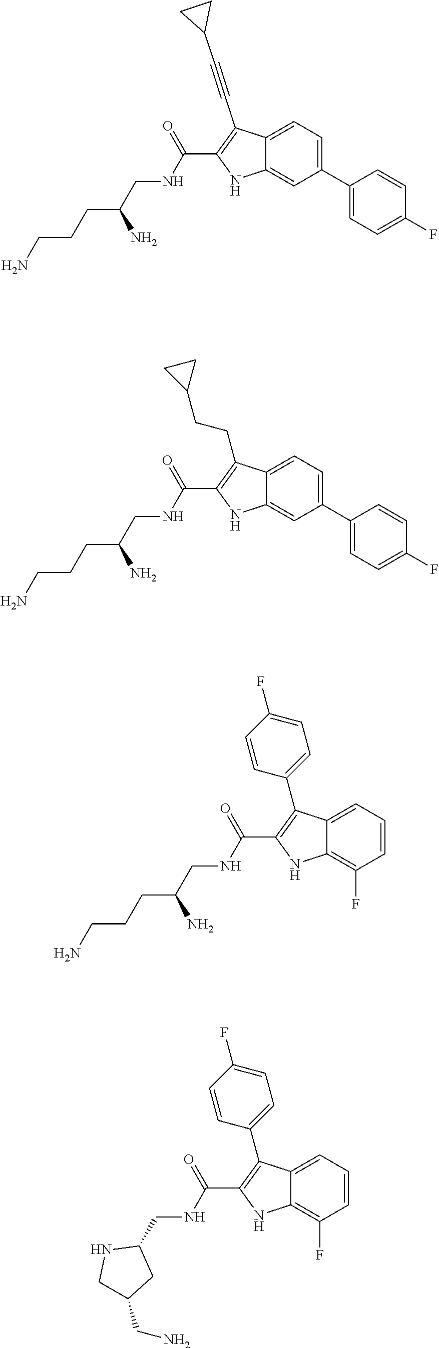 [SMILES: NCCC[C@H](N)CNC(=O)c1[nH]c2cc(-c3ccc(F)cc3)ccc2c1C#CC1CC1 ; NCCC[C@H](N)CNC(=O)c1[nH]c2cc(-c3ccc(F)cc3)ccc2c1CCC1CC1 ; NCCC[C@H](N)CNC(=O)c1[nH]c2c(F)cccc2c1-c1ccc(F)cc1 ; NC[C@@H]1CN[C@H](CNC(=O)c2[nH]c3c(F)cccc3c2-c2ccc(F)cc2)C1]

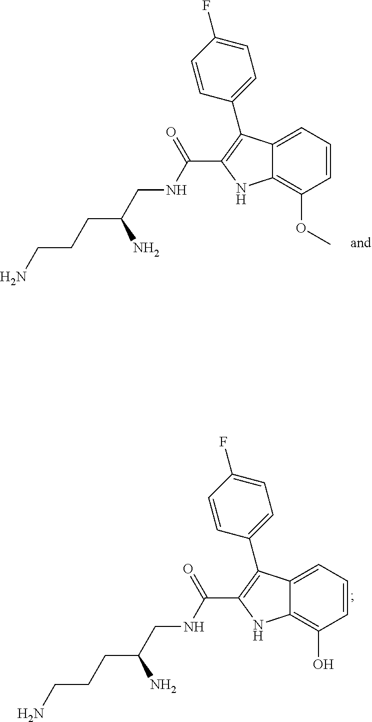 [SMILES: COc1cccc2c(-c3ccc(F)cc3)c(C(=O)NC[C@@H](N)CCCN)[nH]c12 ; NCCC[C@H](N)CNC(=O)c1[nH]c2c(O)cccc2c1-c1ccc(F)cc1]

or a pharmaceutically acceptable salt thereof.

### Compounds of Formula I

It is to understood that the embodiments provided below are for compounds of formula I. It is to be understood the two or more embodiments may be combined.

In one embodiment, the compound of formula I is a compound of formula Ia:

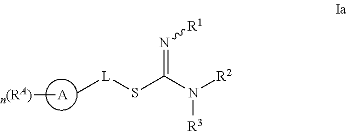

or a salt thereof, wherein:

R¹ is hydrogen, (C₁-C₄)alkyl, or (C₁-C₄)haloalkyl;

R² is hydrogen, (C₁-C₄)alkyl, or (C₁-C₄)haloalkyl;

R³ is hydrogen, (C₁-C₄)alkyl, or (C₁-C₄)haloalkyl;

L is (C₁-C₅)alkylene that is optionally substituted with one or more R^{L} groups;

each R^{L} is independently selected from hydrogen, halo, hydroxy, nitro, cyano, or (C₁-C₄)alkoxy; or any two R^{L} groups that are attached to the same carbon taken together form a (C₃-C₆)carbocycle;

A is aryl or heteroaryl;

each R^{A} is independently selected from the group consisting of halo, cyano, nitro, (C₁-C₆)alkyl, (C₁-C₆)alkoxy, —NR^{X}R^{Y}, aryl or heteroaryl; wherein the (C₁-C₆)alkyl and (C₁-C₆)alkoxy is optionally substituted with one or more groups selected from oxo, halo, hydroxy, (C₁-C₄)alkoxy, nitro, cyano, or —NR^{X}R^{Y}; wherein the aryl and heteroaryl is optionally substituted with one or more groups selected from halo, hydroxy, (C₁-C₄)alkyl, (C₁-C₄)haloalkyl, (C₁-C₄)alkoxy, nitro, cyano, or —NR^{X}R^{Y};

each R^{X} and R^{Y} are independently hydrogen or (C₁-C₄)alkyl; or R^{X} and R^{Y} taken together with the nitrogen to which they are attached form pyrrolidinyl, piperidinyl, piperazinyl, or morpholinyl; and

n is 0, 1, 2, 3, or 4.

In one embodiment, the compound of formula I is a compound of formula Ib:

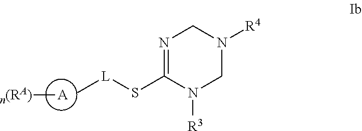

or a salt thereof, wherein:

R³ is hydrogen, (C₁-C₄)alkyl, or (C₁-C₄)haloalkyl;

R⁴ is hydrogen, (C₁-C₆)alkyl, aryl or heteroaryl, wherein the (C₁-C₆)alkyl is optionally substituted with one or more groups selected from halo, hydroxy, nitro, cyano, (C₁-C₄)alkoxy, —NR^{X}R^{Y}, aryl, heteroaryl, aryloxy, or heteroaryloxy, wherein any ary, heteroaryl, aryloxy and heteroaryloxy is optionally substituted with one or more groups selected from halo, hydroxy, (C₁-C₄)alkyl, (C₁-C₄)haloalkyl, (C₁-C₄)alkoxy, nitro, cyano, or —NR^{X}R^{Y};

L is (C₁-C₅)alkylene that is optionally substituted with one or more R^{L} groups;

each R^{L} is independently selected from hydrogen, halo, hydroxy, nitro, cyano, or (C₁-C₄)alkoxy; or any two R^{L} groups that are attached to the same carbon taken together form a (C₃-C₆)carbocycle;

A is aryl or heteroaryl;

each R^{A} is independently selected from the group consisting of halo, cyano, nitro, (C₁-C₆)alkyl, (C₁-C₆)alkoxy, —NR^{X}R^{Y}, aryl or heteroaryl; wherein the (C₁-C₆)alkyl and (C₁-C₆)alkoxy is optionally substituted with one or more groups selected from oxo, halo, hydroxy, (C₁-C₄)alkoxy, nitro, cyano, or —NR^{X}R^{Y}; wherein the aryl and heteroaryl is optionally substituted with one or more groups selected from halo, hydroxy, (C₁-C₄)alkyl, (C₁-C₄)haloalkyl, (C₁-C₄)alkoxy, nitro, cyano, or —NR^{X}R^{Y};

each R^{X} and R^{Y} are independently hydrogen or (C₁-C₄)alkyl; or R^{X} and R^{Y} taken together with the nitrogen to which they are attached form pyrrolidinyl, piperidinyl, piperazinyl or morpholinyl; and

n is 0, 1, 2, 3, or 4.

In one embodiment, wherein R¹ is hydrogen.

In one embodiment, R² is hydrogen.

In one embodiment, R³ is hydrogen or methyl.

In one embodiment, R³ is hydrogen.

In one embodiment, R⁴ is (C₁-C₆)alkyl, wherein the (C₁-C₆)alkyl is optionally substituted with one or more groups selected from halo, hydroxy, halo, nitro, cyano, (C₁-C₄)alkoxy, or phenyl, wherein the phenyl is optionally substituted with one or more groups selected from halo, hydroxy, (C₁-C₄)alkyl, (C₁-C₄)haloalkyl, (C₁-C₄)alkoxy, nitro or cyano.

In one embodiment, R⁴ is methyl or (3,4-dimethoxyphenyl)ethyl.

In one embodiment, R⁴ is methyl.

In one embodiment, the compound of formula I is a compound of formula Ic:

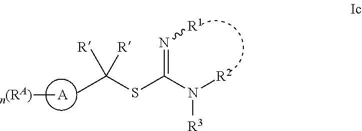

or a salt thereof, wherein each R′ is independently selected from hydrogen, halo, hydroxy, (C₁-C₄)alkyl, (C₁-C₄)haloalkyl, or (C₁-C₄)alkoxy; or any two R′ groups that are attached to the same carbon taken together form a (C₃-C₆)carbocycle.

In one embodiment, each R′ is independently selected from hydrogen, fluoro, methyl, ethyl, isopropyl, fluoromethyl, or trifluoromethyl; or any two R′ groups that are attached to the same carbon taken together form cyclopropyl.

In one embodiment, L is —CH₂—, —CH₂CH₂—, —CH(CH₃)—, —CF₂—, —C(CH₃)₂—, —CH(CH(CH₃)₂)—, —CH(CH₂CH₃)—, —CH(CF₃)—, —CH(CH₂F)—, or

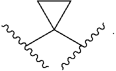 [SMILES: *C1(*)CC1]

In one embodiment, L is —CH₂—, —CH₂CH₂—, or —CH(CH₃)—.

In one embodiment, A is phenyl, naphthyl, 5-6 membered monocyclic heteroary, or 9-10 membered bicyclic heteroaryl.

In one embodiment, when the compound is a compound of formula Ia and A is phenyl, then A is substituted with at least one phenyl group.

In one embodiment, A is naphthyl, 5-6 membered monocyclic heteroary, or 9-10 membered bicyclic heteroaryl.

In one embodiment, A is selected from the group consisting of:

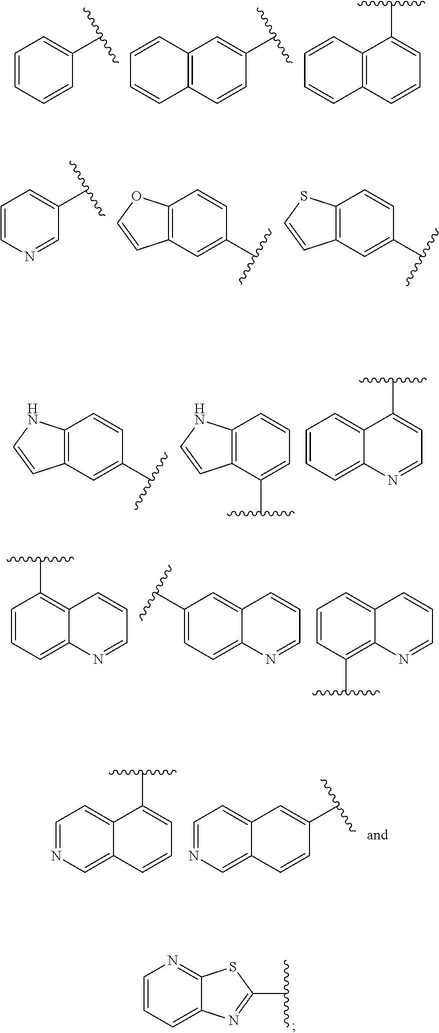

wherein A is optionally substituted with 0, 1, 2, 3, or 4 R^{A} groups.

In one embodiment, A is selected from the group consisting of:

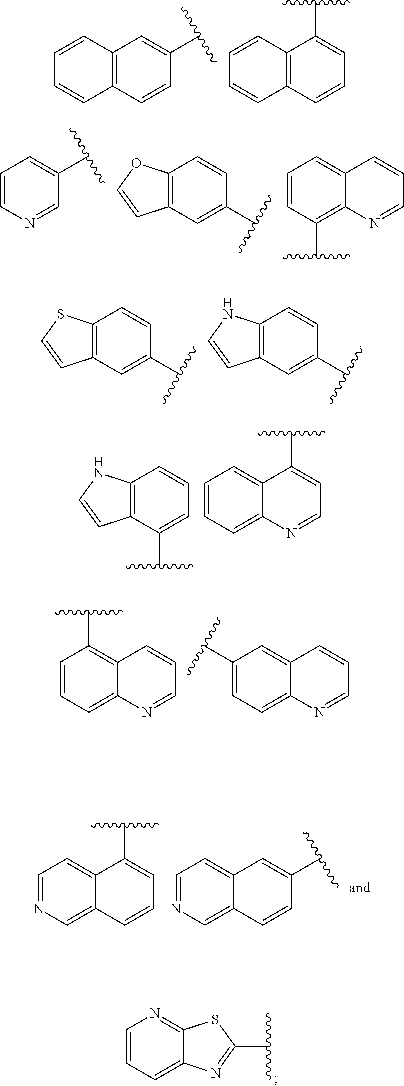

wherein A is optionally substituted with 0, 1, 2, 3, or 4 R^{A} groups.

In one embodiment, each R^{A} is independently selected from the group consisting of halo, (C₁-C₆)alkyl, or phenyl, wherein the (C₁-C₆)alkyl is optionally substituted with one or more groups selected from halo, oxo, or (C₁-C₆)alkoxyl, wherein the phenyl is optionally substituted with one or more halo groups.

In one embodiment, each R^{A} is independently selected from the group consisting of chloro, bromo, fluoro, 4-fluorophenyl, trifluoromethyl, methyl, and tert-butyloxycarbonyl.

In one embodiment, n is 0, 1, or 2.

In one embodiment, the group

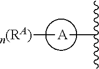

is selected from the group consisting of:

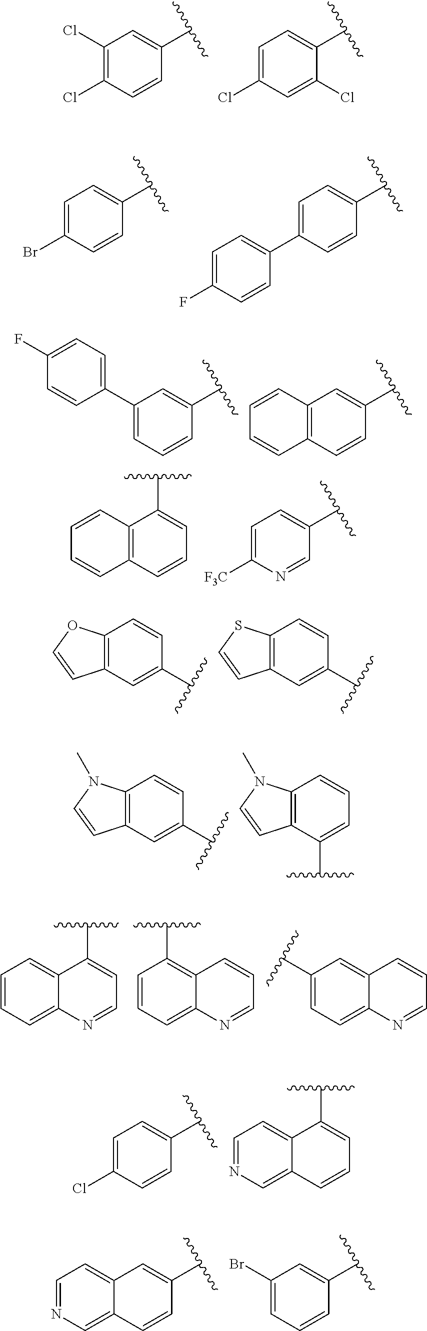

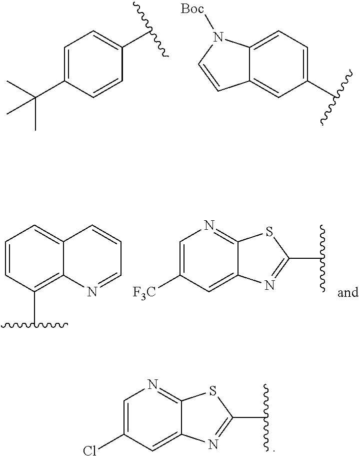

In one embodiment, the group

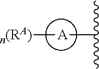

is selected from the group consisting of:

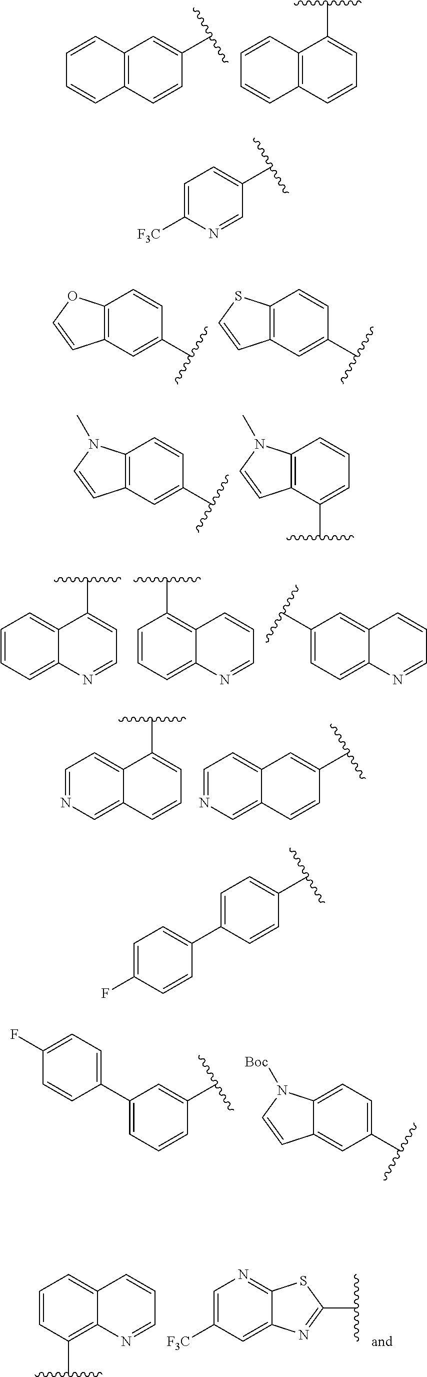

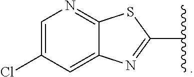 [SMILES: *c1nc2cc(Cl)cnc2s1 ; "."]

In one embodiment, the compound of formula I is selected from the group consisting of:

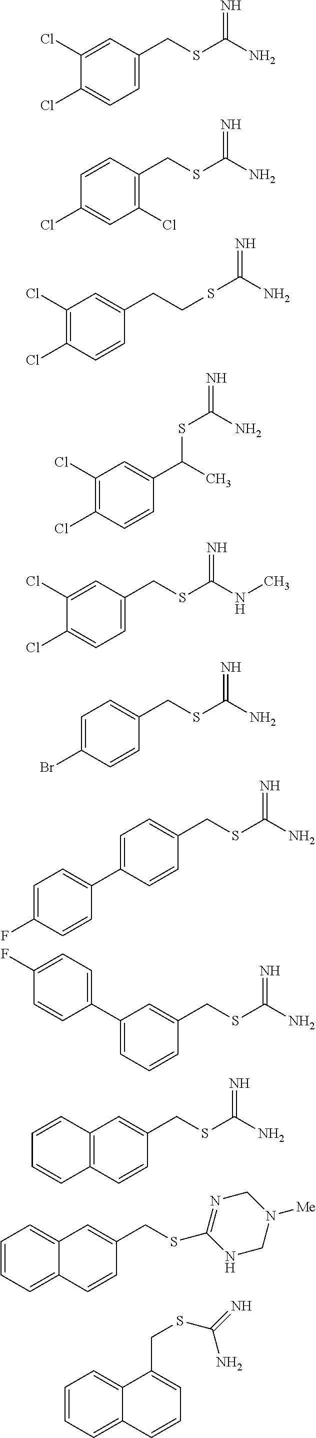

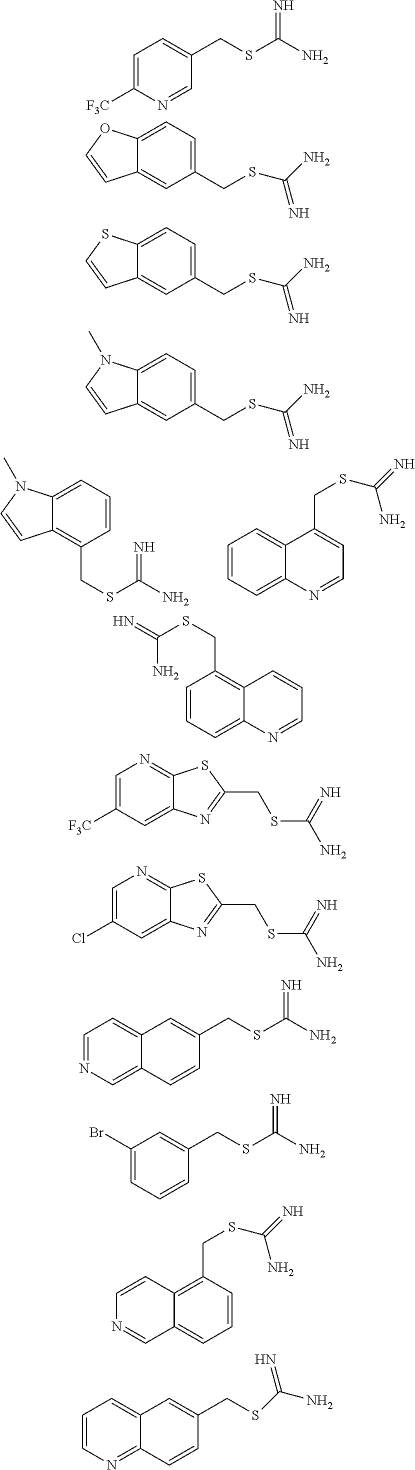

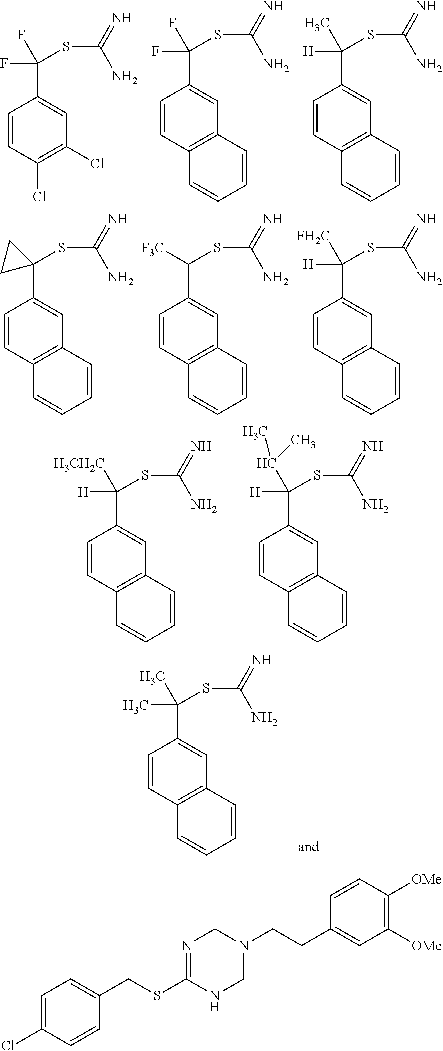

or a salt thereof.

In one embodiment, the compound of formula I is selected from the group consisting of:

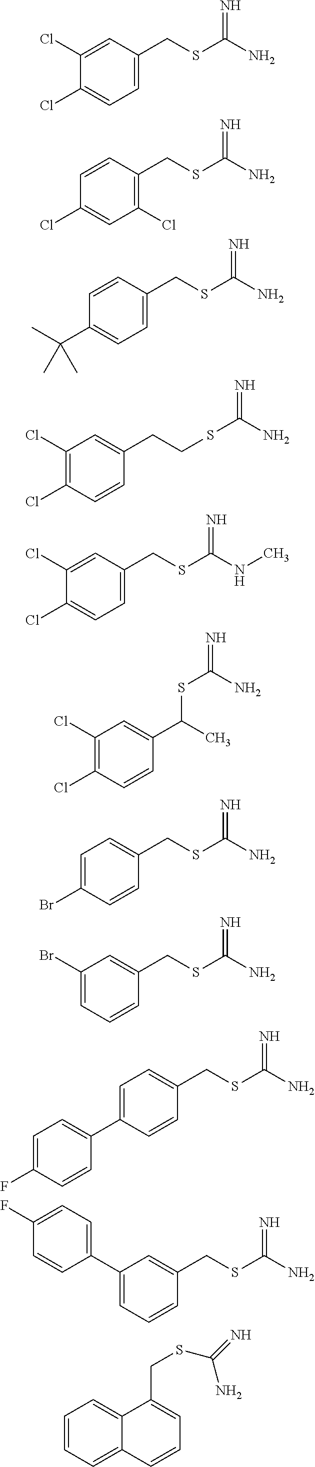

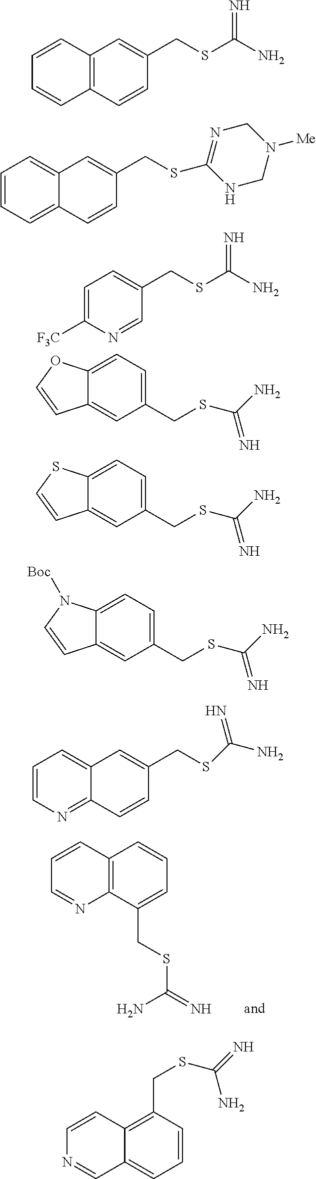

or a salt thereof.

In one embodiment, the compound of formula I is selected from the group consisting of:

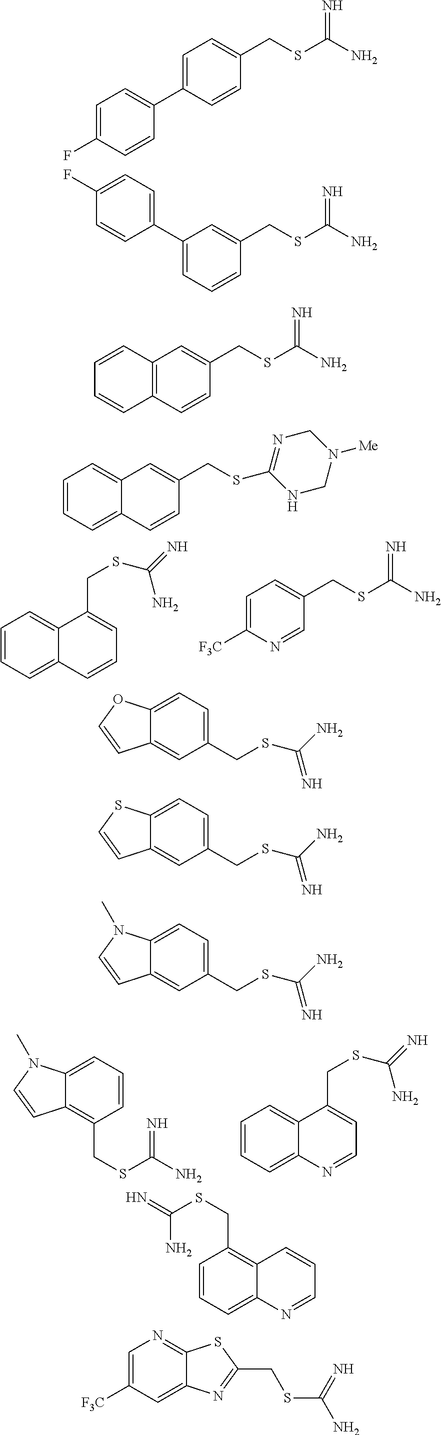

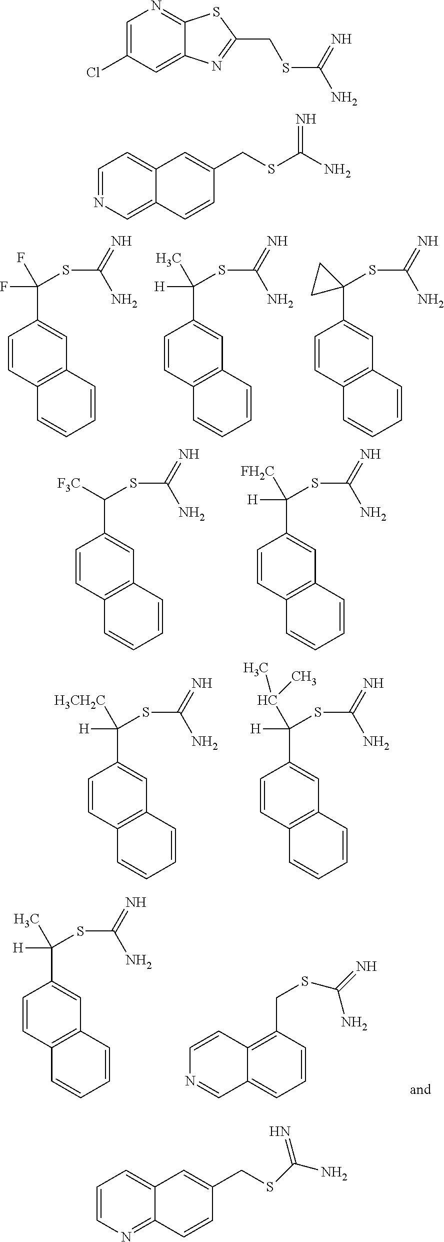

or a salt thereof.

In one embodiment, the compound of formula I is selected from the group consisting of:

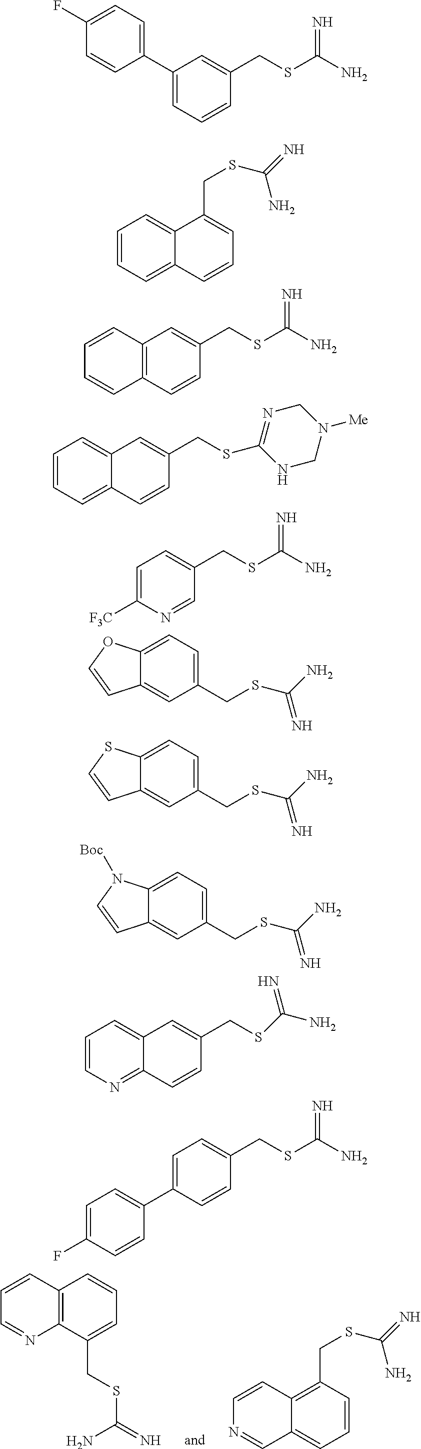

or a salt thereof.

In one embodiment, the compound of formula I is:

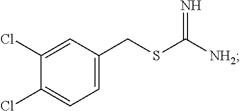 [SMILES: N=C(N)SCc1ccc(Cl)c(Cl)c1]

or a salt thereof.

In one embodiment, the compound of formula I is not:

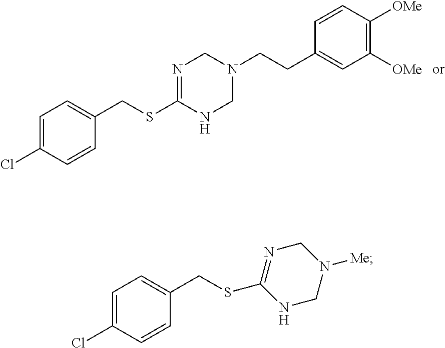 [SMILES: COc1ccc(CCN2CN=C(SCc3ccc(Cl)cc3)NC2)cc1OC ; CN1CN=C(SCc2ccc(Cl)cc2)NC1]

or a salt thereof.

As used herein, the term “minimum inhibitory concentration (MIC)” refers to the lowest concentration of a compound (e.g., an antibiotic) that prevents visible growth of a bacterium. Assays for measuring the MIC of a compound are known in the art, for example, as described herein. As used herein, the term “intrinsic MIC” refers the MIC of a compound (e.g., an antibiotic) for the particular bacterial species that has not been pre-exposed to the compound.

As used herein, the term “sub-inhibitory concentration” refers to a concentration of the antibiotic that does not reduce the visible growth of the bacteria. In certain embodiments, the sub-inhibitory concentration is ½×MIC of the antibiotic. In certain embodiments, the sub-inhibitory concentration of the antibiotic is a concentration that is capable of inducing the expression of one or more efflux pumps in the bacteria.

As used herein, the term “inhibitory concentration” refers to a concentration of the antibiotic that reduces the visible growth of the bacteria. In certain embodiments, this concentration is the intrinsic MIC of the antibiotic.

In certain embodiments, the combination of the compound of formula I and a bacterial efflux pump inhibitor is a synergistic combination.

In certain embodiments, the animal is a non-human animal. For example, in certain embodiments, the animal is a mouse.

Generally, compounds of formula I as well as synthetic intermediates that can be used for preparing compounds of formula I can be prepared as illustrated in the following General Methods and Schemes. It is understood that variable groups shown below (e.g., R¹, R², R³, R⁴, and R^{A}) can represent the final corresponding groups present in a compound of formula I or that these groups can represent groups that can be converted to the final corresponding groups present in a compound of formula I at a convenient point in a synthetic sequence. For example, the variable groups can contain one or more protecting groups that can be removed at a convenient point in a synthetic sequence to provide the final corresponding groups in the compound of formula I. In one embodiment, R and R′ in schemes 1-3 are independently hydrogen, halo, hydroxyl, nitro, cyano, (C₁-C₃)alkyl, or (C₁-C₄)alkoxy. In one embodiment, R and R′ are independently hydrogen or methyl.

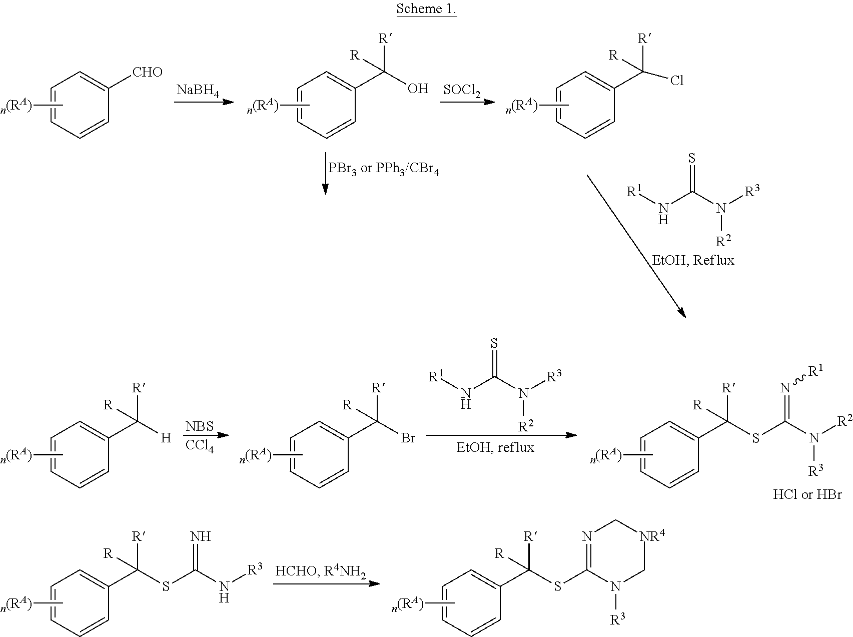

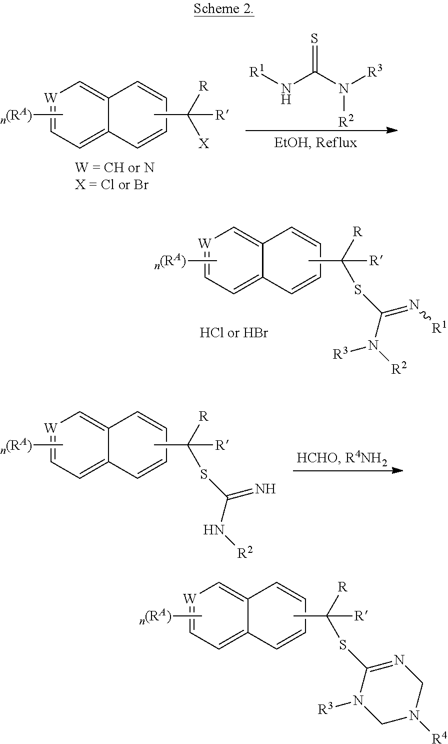

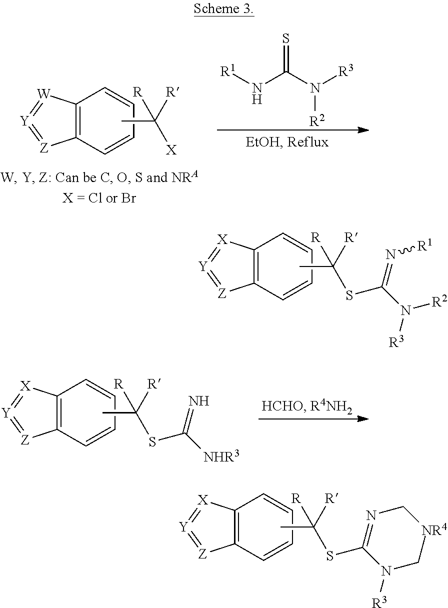

The compounds disclosed herein may be useful for treating bacterial infections (e.g., gram negative and gram positive) when administered with an bacterial efflux pump inhibitor.

In one embodiment the bacterial infection being treated is a Gram-negative bacterial strain infection. In one embodiment the Gram-negative bacterial strain is selected from the group consisting of Acinetobacter baumannii, Acinetobacter calcoaceticus, Acinetobacter haemolyticus, Acinetobacter lwoffi, Actinobacillus actinomycetemcomitans, Aeromonas hydrophilia, Aggregatibacter actinomycetemcomitans, Agrobacterium tumefaciens, Bacteroides distasonis, Bacteroides eggerthii, Bacteroides forsythus, Bacteroides fragilis, Bacteroides ovalus, Bacteroides splanchnicus, Bacteroides thetaiotaomicron, Bacteroides uniformis, Bacteroides vulgatus, Bordetella bronchiseptica, Bordetella parapertussis, Bordetella pertussis, Borrelia burgdorferi, Branhamella catarrhalis, Burkholderia cepacia, Campylobacter coli, Campylobacter fetus, Campylobacterjejuni, Caulobacter crescentus, Chlamydia trachomatis, Citrobacter diversus, Citrobacter freundii, Enterobacter aerogenes, Enterobacter asburiae, Enterobacter cloacae, Enterobacter sakazakii, Escherchia coli, Francisella tularensis, Fusobacterium nucleatum, Gardnerella vaginalis, Haemophilus ducreyi, Haemophilus haemolyticus, Haemophilus influenzae, Haemophilus parahaemolyticus, Haemophilus parainfluenzae, Helicobacter pylori, Kingella denitrificans, Kingella indologenes, Kingella kingae, Kingella oralis, Klebsiella oxytoca, Klebsiella pneumoniae, Klebsiella rhinoscleromatis, Legionella pneumophila, Listeria monocytogenes, Moraxella bovis, Moraxella catarrhalis, Moraxella lacunata, Morganella morganii, Neisseria gonorrhoeae, Neisseria meningitidis, Pantoea agglomerans, Pasteurella canis, Pasteurella haemolytica, Pasteurella multocida, Pasteurella tularensis, Porphyromonas gingivalis, Proteus mirabilis, Proteus vulgaris, Providencia alcalifaciens, Providencia rettgeri, Providencia stuartii, Pseudomonas acidovorans, Pseudomonas aeruginosa, Pseudomonas alcaligenes, Pseudomonas fluorescens, Pseudomonas putida, Salmonella enteriditis, Salmonella paratyphi, Salmonella typhi, Salmonella typhimurium, Serratia marcescens, Shigella dysenteriae, Shigella jlexneri, Shigella sonnei, Stenotrophomonas maltophilla, Veillonella parvula, Vibrio cholerae, Vibrio parahaemolyticus, Yersinia enterocolitica, Yersinia intermedia, Yersinia pestis and Yersinia pseudotuberculosis.

In one embodiment the bacterial infection being treated is a Gram-positive bacterial strain infection. In one embodiment the Gram-positive bacterial strain is selected from the group consisting of Actinomyces naeslundii, Actinomyces viscosus, Bacillus anthracis, Bacillus cereus, Bacillus subtilis, Clostridium difficile, Corynebacterium diphtheriae, Corynebacterium ulcerans, Enterococcus faecalis, Enterococcus faecium, Micrococcus luteus, Mycobacterium avium, Mycobacterium intracellulare, Mycobacterium leprae, Mycobacterium tuberculosis, Propionibacterium acnes, Staphylococcus aureus, Staphylococcus epidermidis, Staphylococcus haemolyticus, Staphylococcus hominis, Staphylococcus hyicus, Staphylococcus intermedius, Staphylococcus saccharolyticus, Staphylococcus saprophyticus, Streptococcus agalactiae, Streptococcus mutans, Streptococcus pneumoniae, Streptococcus pyogenes, Streptococcus salivarius and Streptococcus sanguis.

In one embodiment, the animal is infected with P. aeruginosa.

The compositions can, if desired, also contain other active therapeutic agents, such as a narcotic, a non-steroid anti-inflammatory drug (NSAID), an analgesic, an anesthetic, a sedative, a local anesthetic, a neuromuscular blocker, an anti-cancer, an antimicrobial (for example, an aminoglycoside, an antifungal, an antiparasitic, an antiviral, a carbapenem, a cephalosporin (e.g., cefepime), a fluoroquinolone, a macrolide, a penicillin, a sulfonamide, a tetracycline, another antimicrobial), an anti-psoriatic, a corticosteriod, an anabolic steroid, a diabetes-related agent, a mineral, a nutritional, a thyroid agent, a vitamin, a calcium-related hormone, an antidiarrheal, an anti-tussive, an anti-emetic, an anti-ulcer, a laxative, an anticoagulant, an erythropoietin (for example, epoetin alpha), a filgrastim (for example, G-CSF, Neupogen), a sargramostim (GM-CSF, Leukine), an immunization, an immunoglobulin, an immunosuppressive (for example, basiliximab, cyclosporine, daclizumab), a growth hormone, a hormone replacement drug, an estrogen receptor modulator, a mydriatic, a cycloplegic, an alkylating agent, an anti-metabolite, a mitotic inhibitor, a radiopharmaceutical, an anti-depressant, an anti-manic agent, an anti-psychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog thereof, dornase alpha (Pulmozyme), a cytokine, or any combination thereof.

It will be appreciated that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

When a bond in a compound formula herein is drawn in a non-stereochemical manner (e.g. flat), the atom to which the bond is attached includes all stereochemical possibilities. When a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted unless otherwise noted. In one embodiment, the compound may be at least 51% the absolute stereoisomer depicted. In another embodiment, the compound may be at least 60% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95 the absolute stereoisomer depicted. In another embodiment, the compound may be at least 99% the absolute stereoisomer depicted.

It will also be appreciated by those skilled in the art that certain compounds of the invention can exist in more than one tautomeric form. For example, a substituent of formula —NH—C(═O)H in a compound of formula I could exist in tautomeric form as —N═C(OH)H. The present invention encompasses all tautomeric forms of a compound of formula I as well as mixtures thereof that can exist in equilibrium with non-charged and charged entities depending upon pH, which possess the useful properties described herein

In cases where compounds are sufficiently basic or acidic, a salt of a compound of formula I can be useful as an intermediate for isolating or purifying a compound of formula I. Additionally, administration of a compound of formula I as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartrate, succinate, fumarate, benzoate, ascorbate, α-ketoglutarate, and α-glycerophosphate. Suitable inorganic salts may also be formed, including hydrochloride, sulfate, nitrate, bicarbonate, and carbonate salts. Salts may be obtained using standard procedures well known in the art, for example by reacting a sufficiently basic compound such as an amine with a suitable acid affording the corresponding anion. Alkali metal (for example, sodium, potassium or lithium) or alkaline earth metal (for example calcium) salts of carboxylic acids can also be made.

Pharmaceutically suitable counterions include pharmaceutically suitable cations and pharmaceutically suitable anions that are well known in the art. Examples of pharmaceutically suitable anions include, but are not limited to those described above (e.g. physiologically acceptable anions) including Cl⁻, Br⁻, I⁻, CH₃SO₃⁻, H₂PO₄⁻, CF₃SO₃⁻, p-CH₃C₆H₄ SO₃⁻, citrate, tartrate, phosphate, malate, fumarate, formate, or acetate.

It will be appreciated by those skilled in the art that a compound of the invention comprising a counterion can be converted to a compound of the invention comprising a different counterion. Such a conversion can be accomplished using a variety of well-known techniques and materials including but not limited to ion exchange resins, ion exchange chromatography and selective crystallization.

The compounds of formula I can be formulated as pharmaceutical compositions and administered to a mammalian host, such as a human patient in a variety of forms adapted to the chosen route of administration, i.e., orally or parenterally, by intravenous, intramuscular, topical or subcutaneous routes. For oral administration the compounds can be formulated as a solid dosage form with or without an enteric coating.

Thus, the present compounds may be systemically administered, e.g., orally, in combination with a pharmaceutically acceptable vehicle such as an inert diluent, excipient or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 90% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations, particles, and devices.

The active compound may also be administered intravenously or intramuscularly by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina, nanoparticles, and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 1 to about 500 mg/kg, e.g., from about 5 to about 400 mg/kg of body weight per day, such as 1 to about 250 mg per kilogram body weight of the recipient per day.

The compound is conveniently formulated in unit dosage form; for example, containing 5 to 500 mg, 10 to 400 mg, or 5 to 100 mg of active ingredient per unit dosage form. In one embodiment, the invention provides a composition comprising a compound of the invention formulated in such a unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

Co-administration of a compound disclosed herein with one or more other active therapeutic agents (e.g., antibacterial agents) generally refers to simultaneous or sequential administration of a compound disclosed herein and one or more other active therapeutic agents, such that therapeutically effective amounts of disclosed herein and one or more other active therapeutic agents are both present in the body of the patient.

The ability of a compound to produce an antibiotic effect can be determined using a method as described in Example 21. Data for representative compounds used in combination with the efflux pump inhibitor [(2S,4R)-4-(ammoniomethyl)-2-((6-(4-fluorophenyl)-1H-indole-2-carboxamido)methyl)pyrrolidin-1-ium] is shown in Table 1.

TABLE 1

| | | Antibiotic Activity | | Antibiotic Activity | |
|---|---|---|---|---|---|
| | | Against P. aeruginosa^{a} | | Against E. coli^{b} | |
| | | | MIC | | MIC |
| | | Intrinsic | with EPI* | Intrinsic | with EPI* |
| | | MIC | μg/ml | MIC | μg/ml |
| Example | Structure | (μ/mL) | (Enhancement) | (μg/mL) | (Enhancement) |
| 1 | 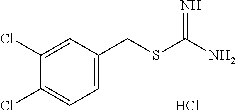 [SMILES: N=C(N)SCc1ccc(Cl)c(Cl)c1 ; Cl] | >256 | 0.5 | 64 | 4 |
| 2 | 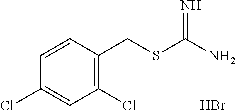 [SMILES: N=C(N)SCc1ccc(Cl)cc1Cl ; Br] | >256 | 1.0 | >256 | >256 |
| 3 | 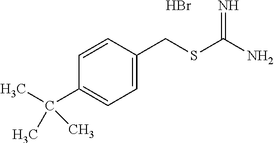 | >256 | 256 | 256 | 256 |
| 4 | 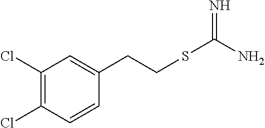 [SMILES: N=C(N)SCCc1ccc(Cl)c(Cl)c1] | >256 | >256 | 256 | 256 |
| 5 | 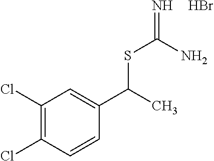 | >256 | 4.0 | 256 | 256 |
| 6 | 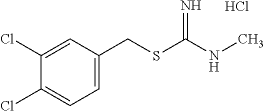 | >256 | 4.0 | >256 | >256 |
| 7 | 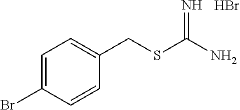 [SMILES: N=C(N)SCc1ccc(Br)cc1 ; Br] | >256 | 1.0 | >256 | >256 |
| 8 | 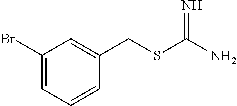 [SMILES: N=C(N)SCc1cccc(Br)c1] | >256 | >256 | >256 | >256 |
| 9 | 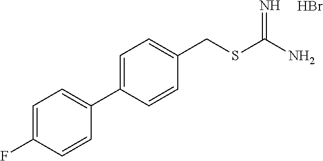 [SMILES: N=C(N)SCc1ccc(-c2ccc(F)cc2)cc1 ; Br] | >256 | >256 | 64 | 64 |
| 10 | 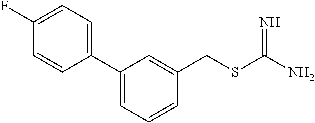 [SMILES: N=C(N)SCc1cccc(-c2ccc(F)cc2)c1] | >256 | >256 | 64 | 64 |
| 11 | 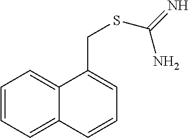 [SMILES: N=C(N)SCc1cccc2ccccc12] | >256 | >256 | >256 | >256 |
| 12 | 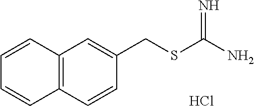 [SMILES: N=C(N)SCc1ccc2ccccc2c1 ; Cl] | >256 | 4 | >256 | 16 |
| 13 | 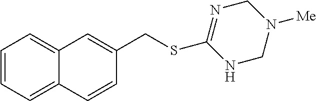 [SMILES: CN1CN=C(SCc2ccc3ccccc3c2)NC1] | >256 | 16 | >256 | 16 |
| 14 | 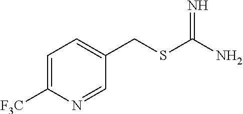 [SMILES: N=C(N)SCc1ccc(C(F)(F)F)nc1] | >256 | >256 | >256 | >256 |
| 15 | 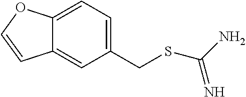 [SMILES: N=C(N)SCc1ccc2occc2c1] | 16 | 4 | >256 | >256 |
| 16 | 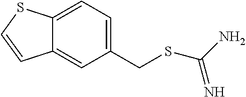 [SMILES: N=C(N)SCc1ccc2sccc2c1] | >256 | 4 | >256 | >256 |
| 17 | 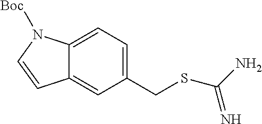 [SMILES: CC(C)(C)OC(=O)n1ccc2cc(CSC(=N)N)ccc21] | >256 | >256 | >128 | 128 |
| 18 | 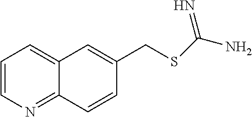 [SMILES: N=C(N)SCc1ccc2ncccc2c1] | >256 | >256 | >256 | >256 |
| 19 | 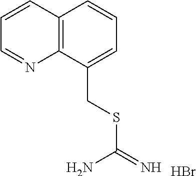 [SMILES: N=C(N)SCc1cccc2cccnc12 ; Br] | >256 | >256 | >256 | >256 |
| 20 | 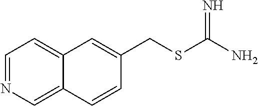 [SMILES: N=C(N)SCc1ccc2cnccc2c1] | >256 | >256 | >256 | >256 |

^{a}P. aeruginosa PAO1
^{b}E. coli 25922
*The EPI that was used in these assay was (2S,4R)-4-(ammoniomethyl)-2-((6-(4-fluoropheny1)-1H-indole-2-carboxamido)methyl)pyrrolidin-1-ium. The MIC₉₀ was determined by using varying concentration of each test compounds with and without 12.5 ug/ml of bacterial efflux pump inhibitor.

The invention will now be illustrated by the following non-limiting examples.

### EXAMPLES

### Example 1. Preparation of 3,4-Dichlorobenzyl Carbamimidothioate Hydrobromide

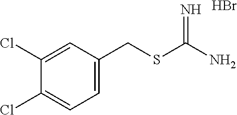 [SMILES: Br ; N=C(N)SCc1ccc(Cl)c(Cl)c1]

### 3,4-Dichlorobenzyl Carbamimidothioate Hydrobromide

4-(Bromomethyl)-1,2-dichlorobenzene (500 mg, 2.09 mmol) and thiourea (133 mg, 1.74 mmol) were dissolved in ethanol (20 mL). The reaction mixture was refluxed for 2 hours. The reaction mixture was cooled, and ethanol was removed under reduced pressure. The resulting residue was suspended in dichloromethane. The suspension was filtered to give a product as a white solid (473 mg, 86%); ¹H NMR (300 MHz) (DMSO-d₆) δ 9.23 (s, 1H), 9.04 (s, 1H), 7.72 (s, 1H), 7.72-7.64 (d, 1H), 7.44-7.41 (d, 1H), 4.51 (s, 2H); LC/MS: Rt=2.499, M+H⁺=236.

### Example 2. Preparation of 2,4-Dichlorobenzyl Carbamimidothioate Hydrobromide

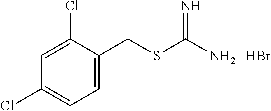 [SMILES: N=C(N)SCc1ccc(Cl)cc1Cl ; Br]

### 2,4-Dichlorobenzyl Carbamimidothioate Hydrobromide

1-(Bromomethyl)-2,4-dichlorobenzene (500 mg, 2.08 mmol) and thiourea (132 mg, 1.73 mmol) were dissolved in ethanol (20 mL). The reaction mixture was refluxed for 2 hours. The reaction mixture was cooled, and ethanol was removed under reduced pressure. The resulting residue was suspended in dichloromethane. The suspension was filtered to give a product as a white solid (441 mg, 81%); ¹H NMR (300 MHz) (DMSO-d₆) δ 9.14 (bs, 4H), 7.72 (d, J=2 Hz, 1H), 7.58 (d, J=9 Hz, 1H), 7.48 (dd, J=8 Hz, J=2 Hz, 1H), 4.54 (s, 2H); LC/MS RT=2.46 (M+H⁺: 235/237).

### Example 3. Preparation of 4-(t-Butyl)Benzyl Carbamimidothioate Hydrobromide

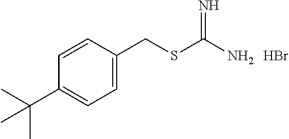

### 4-(t-Butyl)Benzyl Carbamimidothioate Hydrobromide

1-(Bromomethyl)-4-(t-butyl)benzene (300 mg, 1.32 mmol) and thiourea (77 mg, 1.02 mmol) were dissolved in ethanol (20 mL). The reaction mixture was refluxed for 2 hours. The reaction mixture was cooled, and ethanol was removed under reduced pressure. The resulting residue was suspended in dichloromethane. The suspension was filtered to give a product as a white solid (130 mg, 42%); ¹H NMR (300 MHz) (DMSO-d₆) δ 9.12 (bs, 2H), 8.94 (bs, 2H), 7.38 (dd, J=9 Hz, J=2 Hz, 2H), 7.31 (d, J=8 Hz, 2H), 4.42 (s 2H), 1.25 (s, 9H); LC/MS RT=2.62 (M+H⁺: 223).

### Example 4. Preparation of 3,4-dichlorophenethyl carbamimidothioate

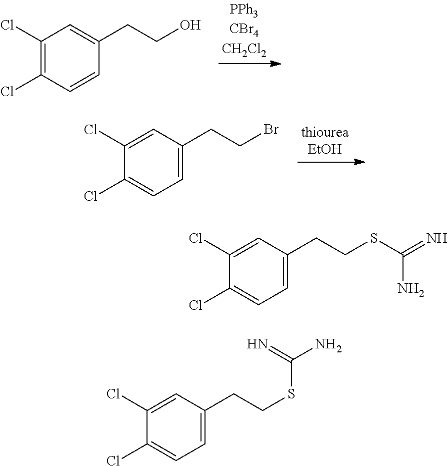

### 3,4-Dichlorophenethyl Carbamimidothioate

4-(2-Bromoethyl)-1,2-dichlorobenzene (219 mg, 0.86 mmol) and thiourea (33 mg, 0.43 mmol) were dissolved in ethanol (10 mL). The reaction mixture was refluxed for 2 hours. The reaction mixture was cooled, and ethanol was removed under reduced pressure. The residue was purified on an ISCO chromatography (100% ethyl acetate followed by 10% methanol/dichloromethane+1% NH₄OH) to give the product as a white solid. (35 mg, 37%); ¹H NMR (300 MHz) (DMSO-d₆) δ 8.97 (bs, 3H), 7.60-7.57 (m, 2H), 7.28 (d, J=7 Hz, 1H), 3.41 (t, J=8 Hz, 2H), 3.12 (t, J=7 Hz, 2H); LC/MS RT=2.56 (M+H⁺: 249/251).

The requisite intermediate was prepared as follows:

### Step 1)

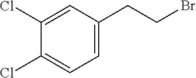 [SMILES: Clc1ccc(CCBr)cc1Cl]

### 4-(2-Bromoethyl)-1,2-dichlorobenzene

2-(3,4-Dichlorophenyl)ethan-1-ol (500 mg, 2.62 mmol), triphenylphosphine (1.03 g, 3.93 mmol) and tetrabromomethane (1.30 g, 3.93 mmol) were dissolved in dichloromethane (20 mL). The reaction mixture was stirred for overnight in the room temperature. The reaction mixture was concentrated under reduced pressure. The residue was purified on an ISCO chromatography (0-10% ethyl acetate/hexane) to give the product as colorless oil (219 mg, 33%); ¹H NMR (300 MHz) (CDCl₃) δ 7.39 (d, J=8 Hz, 1H), 7.31 (d, J=2 Hz, 1H), 7.06 (dd, J=8 Hz, J=2 Hz, 1H), 3.54 (t, J=7 Hz, 2H), 3.12 (t, J=7 Hz, 2H).

### Example 5. Preparation of 1-(3,4-Dichlorophenyl)ethyl Carbamimidothioate Hydrobromide

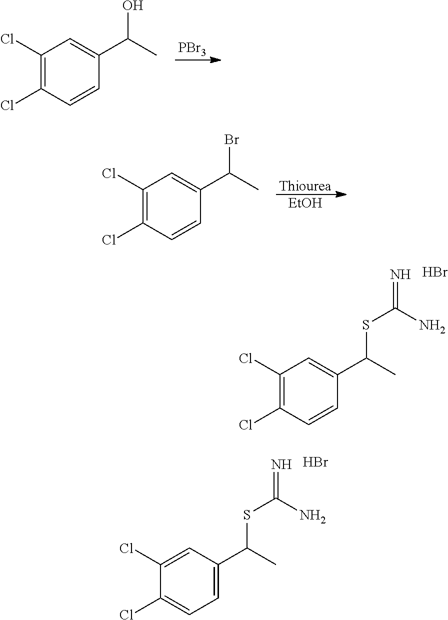

### 1-(3,4-Dichlorophenyl)ethyl Carbamimidothioate Hydrobromide

4-(1-Bromoethyl)-1,2-dichlorobenzene (412 mg, 1.62 mmol) and thiourea (103 mg, 1.35 mmol) were dissolved in ethanol (20 mL). The reaction mixture was refluxed for 2 hours. The reaction mixture was cooled, and ethanol was removed under reduced pressure. The resulting residue was suspended in dichloromethane. The suspension was filtered to give a product as a white solid (356 mg, 80%); ¹H NMR (300 MHz) (DMSO-d₆) δ 9.27 (s, 2H), 9.04 (s, 2H), 7.76 (s, 1H), 7.68-7.66 (d, 1H), 7.49-7.45 (d, 1H), 5.23-5.21 (q, 1H), 1.62-1.60 (s, 3H); LC/MS RT=2.548 (M+H⁺: 250).

The requisite intermediate was prepared as follows:

### Step 1)

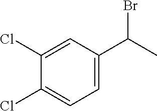

### 4-(1-Bromoethyl)-1,2-dichlorobenzene

1-(3,4-Dichlorophenyl)ethan-1-ol (500 mg, 2.64 mmol) and phosphorus tribromide (850 mg, 3.14 mmol) were dissolved in dichloromethane (20 mL). The reaction mixture was stirred for overnight at room temperature. The reaction mixture was quenched with ice water. The reaction was washed with saturated sodium bicarbonate followed by brine. The organic layer was dried over Na₂SO₄ and concentrated under reduced pressure. The residue was purified on an ISCO chromatograph (0 to 30% ethyl acetate/hexane) to give the product as a white solid (508 mg, 77%); ¹H NMR (300 MHz) (CDCl₃) δ7.53 (s, 1H), 7.43-7.40 (d, 1H), 7.29-7.26 (d, 1H), 5.12-5.07 (q, 1H), 2.03-2.00 (s, 3H).

### Example 6. Preparation of 3,4-dichlorobenzyl Methylcarbamimidothioate Hydrobromide

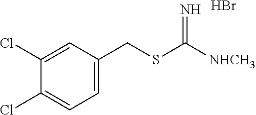 [SMILES: Br ; CNC(=N)SCc1ccc(Cl)c(Cl)c1]

### 3,4-Dichlorobenzyl Methylcarbamimidothioate Hydrobromide

4-(Bromomethyl)-1,2-dichlorobenzene (500 mg, 2.08 mmol) and N-methylthiourea (156 mg, 1.74 mmol) were dissolved in ethanol (20 mL). The reaction mixture was refluxed for 2 hours. The reaction mixture was cooled, and ethanol was removed under reduced pressure. The resulting residue was suspended in dichloromethane. The suspension was filtered to give a product as a white solid (371 mg, 65%); ¹H NMR (300 MHz) (DMSO-d₆) δ 9.77 (s, 1H), 9.47 (s, 1H), 9.15 (s, 1H), 7.71 (s, 1H), 7.63-7.61 (d, 1H), 7.41-7.39 (d, 1H), 4.58 (s, 2H), 2.84 (s, 3H); LC/MS RT=2.492 (M+H⁺: 250).

### Example 7. Preparation of 4-Bromobenzyl Carbamimidothioate Hydrobromide

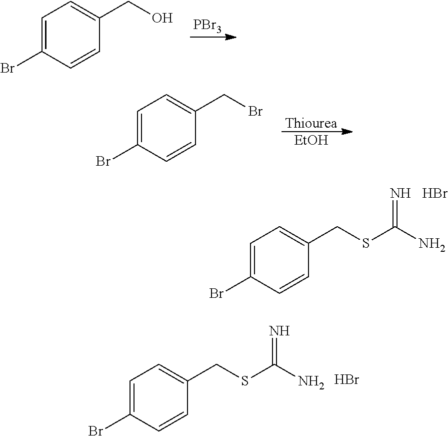

### 4-Bromobenzyl Carbamimidothioate Hydrobromide

1-Bromo-4-(bromomethyl)benzene (507 mg, 2.03 mmol) and thiourea (129 mg, 1.69 mmol) were dissolved in ethanol (20 mL). The reaction mixture was refluxed for 2 hours. The reaction mixture was cooled, and ethanol was removed under reduced pressure. The resulting residue was suspended in dichloromethane. The suspension was filtered to give a product as a white solid (535 mg, 97%); ¹H NMR (300 MHz) (DMSO-d₆) δ 9.21 (s, 2H), 9.01 (s, 2H), 7.59-7.55 (m, 2H), 7.40-7.36 (m, 2H), 4.48 (s, 2H); LC/MS RT=2.406 (M+H⁺: 246).

The requisite intermediate was prepared as follows:

### Step 1)

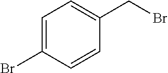

### 1-Bromo-4-(bromomethyl)benzene

(4-Bromophenyl)methanol (1 g, 5.35 mmol) and phosphorus tribromide (0.61 ml, 6.42 mmol) were dissolved in dichloromethane (30 mL). The reaction mixture was stirred for overnight at room temperature. The reaction mixture was quenched with ice water. The reaction mixture was diluted with dichloromethane, and it was washed with saturated sodium bicarbonate followed by brine. The organic layer was dried over Na₂SO₄ and concentrated under reduced pressure to give the crude product as a white solid (1.1 g, 83%). The crude product was used directly without further purification; ¹H NMR (300 MHz) (CDCl₃) δ 7.49-7.46 (m, 2H), 7.28-7.25 (m, 2H), 4.44 (s, 2H).

### Example 8. Preparation of 3-bromobenzyl Carbamimidothioate Hydrobromide

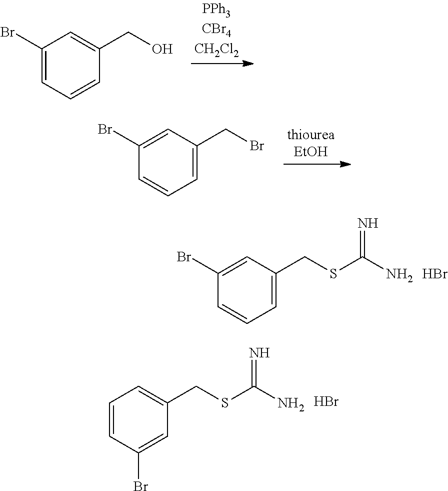

### 3-Bromobenzyl Carbamimidothioate Hydrobromide

1-Bromo-3-(bromomethyl)benzene (750 mg, 3.00 mmol) and thiourea (76 mg, 1.00 mmol) were dissolved in ethanol (20 mL). The reaction mixture was refluxed for 3 hours. The reaction mixture was cooled, and ethanol was removed under reduced pressure. The resulting residue was suspended in dichloromethane. The suspension was filtered to give a product as a white solid (323 mg, 99%); ¹H NMR (300 MHz) (DMSO-d₆) δ 9.17 (bs, 2H), 8.99 (bs, 2H), 7.64 (s, 1H), 7.52 (d, J=8 Hz, 1H), 7.41 (d, J=8 Hz, 1H), 7.34 (t, J=8 Hz, 1H), 4.45 (s, 2H); LC/MS RT=2.40 (M+H⁺: 245/247).

The requisite intermediate was prepared as follows:

### Step 1)

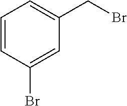

### 1-Bromo-3-(bromomethyl)benzene

(3-Bromophenyl)methanol (1.0 g, 5.35 mmol), triphenylphosphine (2.11 g, 8.03 mmol) and tetrabromomethane (2.66 g, 8.03 mmol) were dissolved in dichloromethane (50 mL). The reaction mixture was stirred overnight in the room temperature. The reaction mixture was concentrated under reduced pressure. The residue was purified on an ISCO chromatography (0-10% ethyl acetate/hexane) to give the product as colorless oil (750 mg, 56%); ¹H NMR (300 MHz) (CDCl₃) δ 7.55 (s, 1H), 7.43 (d, J=8 Hz, 1H), 7.32 (d, J=8 Hz, 1H), 7.22 (t, J=8 Hz, 1H), 4.43 (s, 2H).

### Example 9. Preparation of (4′-fluoro-[1,1′-biphenyl]-4-yl)methyl Carbamimidothioate Hydrobromide

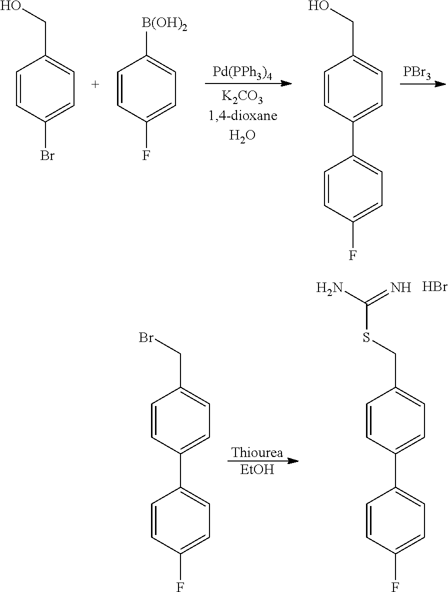

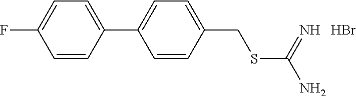 [SMILES: N=C(N)SCc1ccc(-c2ccc(F)cc2)cc1 ; Br]

### (4′-Fluoro-[1,1′-biphenyl]-4-yl)methyl Carbamimidothioate Hydrobromide

4-(Bromomethyl)-4′-fluoro-1,1′-biphenyl (509 mg, 1.92 mmol) and thiourea (121 mg, 1.60 mmol) were dissolved in ethanol (20 mL). The reaction mixture was refluxed for 2 hours. The reaction mixture was cooled, and ethanol was removed under reduced pressure. The resulting residue was suspended in dichloromethane. The suspension was filtered to give a product as a white solid (521 mg, 95%); ¹H NMR (300 MHz) (DMSO-d₆) δ 9.21 (s, 2H), 9.02 (s, 2H), 7.74-7.64 (m, 4H), 7.51-7.48 (d, 2H), 7.32-7.25 (m, 2H), 4.54 (s, 2H); LC/MS RT=2.664 (M+H⁺: 261).

The requisite intermediates were prepared as follows:

### Step 1)

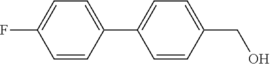 [SMILES: OCc1ccc(-c2ccc(F)cc2)cc1]

### (4′-Fluoro-[1,1′-biphenyl]-4-yl)methanol

(4-Bromophenyl)methanol (2.00 g, 10.7 mmol), 4-fluorophenyl)boronic acid (1.49 g, 10.7 mmol), Pd(PPh₃)₄ (124 mg, 0.11 mmol) and K₂CO₃ (4.43 g, 32.1 mmol) were dissolved in a mixture of dioxane (30 mL) and water (9 mL). The air was evacuated from the reaction flask and replaced with N₂. The reaction mixture was then refluxed overnight. The resulting mixture was diluted with ethyl acetate and washed with water followed by brine. The organic layer was dried over Na₂SO₄ and was concentrated under reduced pressure. The residue was purified on an ISCO chromatograph (0 to 50% ethyl acetate/hexane) to give the product as a white solid (2.20 g, >99%); ¹H NMR (300 MHz) (CDCl₃) δ 7.56-7.54 (m, 4H), 7.45 (d, 2H), 7.13 (t, 2H), 4.75 (s, 2H.

### Step 2)

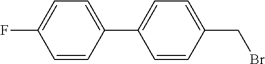 [SMILES: Fc1ccc(-c2ccc(CBr)cc2)cc1]

### 4-(Bromomethyl)-4′-fluoro-1,1′-biphenyl

(4′-Fluoro-[1,1′-biphenyl]-4-yl)methanol (2.20 g, 10.88 mmol) and phosphorus tribromide (1.24 mL, 13.1 mmol) were dissolved in dichloromethane (30 mL). The reaction mixture was stirred for overnight at room temperature. The reaction mixture was quenched with ice water. The reaction mixture was diluted with ethyl acetate, and it was washed with saturated sodium bicarbonate followed by brine. The organic layer was dried over Na₂SO₄ and concentrated under reduced pressure to give the crude product as a white solid (2.1 g, 73%). The crude product was used directly without further purification; ¹H NMR (300 MHz) (CDCl₃) δ7.57-7.45 (m, 6H), 7.16-7.11 (m, 2H), 4.55 (s, 2H).

### Example 10. Preparation of (4′-fluoro-[1,1′-biphenyl]-3-yl)methyl Carbamimidothioate Hydrobromide

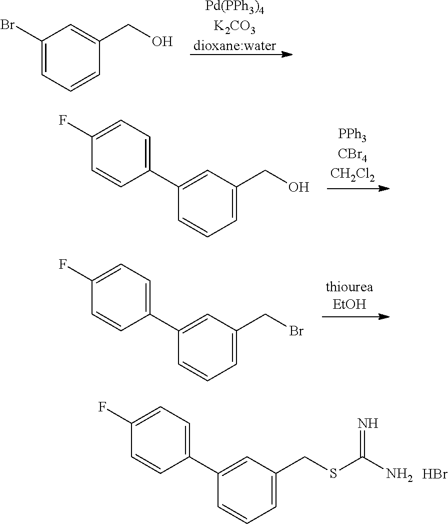

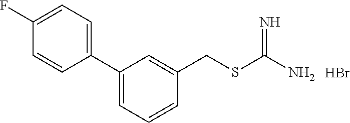 [SMILES: N=C(N)SCc1cccc(-c2ccc(F)cc2)c1 ; Br]

### (4′-Fluoro-[1,1′-biphenyl]-3-yl)methyl Carbamimidothioate Hydrobromide

3-(Bromomethyl)-4′-fluoro-1,1′-biphenyl (249 mg, 0.94 mmol) and thiourea (36 mg, 0.47 mmol) were dissolved in ethanol (10 mL). The reaction mixture was refluxed for 2 hours. The reaction mixture was cooled, and ethanol was removed under reduced pressure. The resulting residue was suspended in dichloromethane. The suspension was filtered to give a product as a white solid (142 mg, 89%); ¹H NMR (300 MHz) (DMSO-d₆) δ 9.04 (bs, 4H), 7.69-7.66 (m, 3H), 7.60 (d, J=9 Hz, 1H), 7.46 (t, J=8 Hz, 1H), 7.39 (d, J=7 Hz, 1H), 7.31 (t, J=9 Hz, 2H), 4.50 (s, 2H); LC/MS RT=2.65 (M+H⁺: 261).

The requisite intermediates were prepared as follows:

### Step 1)

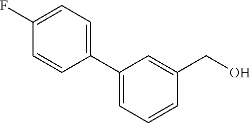 [SMILES: OCc1cccc(-c2ccc(F)cc2)c1]

### (4′-Fluoro-[1,1′-biphenyl]-3-yl)methanol

(3-Bromophenyl)methanol (2.0 g, 10.69 mmol), 4-fluorophenyl)boronic acid (2.24 g, 16.04 mmol), Pd(PPh₃)₄ (618 mg, 0.53 mmol) and K₂CO₃ (4.43 g, 32.07 mmol) were dissolved in a mixture of dioxane (30 mL) and water (10 mL). The air was evacuated from the reaction flask and replaced with N₂. The reaction mixture was then refluxed for 5 hours. Reaction was monitored by TLC and stopped once the starting material was consumed. The reaction mixture was diluted with ethyl acetate, and it was washed with water followed by brine. The organic layer was dried over Na₂SO₄ and was concentrated under reduced pressure. The residue was purified on an ISCO chromatograph (0 to 30% ethyl acetate/hexane) to give the product as brown oil (2.16 g, 100%); ¹H NMR (300 MHz) (CDCl₃) δ 7.58-7.53 (m, 3H), 7.49-7.41 (m, 2H), 7.35 (d, J=8 Hz, 1H), 7.16-7.10 (m, 2H), 4.76 (s, 2H).

### Step 2)

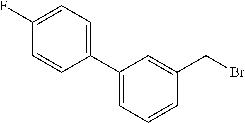 [SMILES: Fc1ccc(-c2cccc(CBr)c2)cc1]

### 3-(Bromomethyl)-4′-fluoro-1,1′-biphenyl

(4′-Fluoro-[1,1′-biphenyl]-3-yl)methanol (1.0 g, 4.94 mmol), triphenylphosphine (1.94 g, 7.41 mmol) and tetrabromomethane (2.46 g, 7.41 mmol) were dissolved in dichloromethane (50 mL). The reaction mixture was stirred overnight in the room temperature. The reaction mixture was concentrated under reduced pressure. The residue was purified on an ISCO chromatography (0-10% ethyl acetate/hexane) to give the product as a white solid (627 mg, 48%); ¹H NMR (300 MHz) (CDCl₃) δ 7.56-7.54 (m, 3H), 7.47 (dd, J=8 Hz, J=2 Hz, 1H), 7.46-7.39 (m, 2H), 7.13 (t, J=8 Hz, 2H), 4.55 (s, 2H).

### Example 11. Preparation of naphthalen-1-ylmethyl Carbamimidothioate Hydrobromide

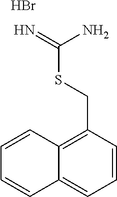 [SMILES: Br ; N=C(N)SCc1cccc2ccccc12]

### Naphthalen-1-ylmethyl Carbamimidothioate Hydrobromide

1-(Bromomethyl)naphthalene (505 mg, 2.28 mmol) and thiourea (144 mg, 1.90 mmol) were dissolved in ethanol (20 mL). The reaction mixture was refluxed for 2 hours. The reaction mixture was cooled, and ethanol was removed under reduced pressure. The resulting residue was suspended in dichloromethane. The suspension was filtered to give product as a white solid (535 mg, 95%); ¹H NMR (300 MHz) (DMSO-d₆) δ 9.24 (s, 2H), 9.05 (s, 2H), 8.19-8.16 (d, 1H), 7.99-7.92 (dd, 2H), 7.65-7.47 (m, 4H), 4.99 (s, 2H); LC/MS RT=2.490 (M+H⁺: 217).

### Example 12. Preparation of naphthalen-2-ylmethyl Carbamimidothioate Hydrobromide

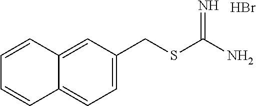 [SMILES: N=C(N)SCc1ccc2ccccc2c1 ; Br]

### Naphthalen-2-ylmethyl Carbamimidothioate Hydrobromide

2-(Bromomethyl)naphthalene (500 mg, 2.26 mmol) and thiourea (143 mg, 1.88 mmol) were dissolved in ethanol (20 mL). The reaction mixture was refluxed for 2 hours. The reaction mixture was cooled, and ethanol was removed under reduced pressure. The resulting residue was suspended in dichloromethane. The suspension was filtered to give product as a white solid (546 mg, 98%); ¹H NMR (300 MHz) (DMSO-d₆) δ 9.23 (s, 2H), 9.00 (s, 2H), 7.95-7.87 (m, 4H), 7.56-7.50 (m, 3H), 4.66 (s, 2H); LC/MS: Rt=2.485, M+H⁺=217.

### Example 13. Preparation of 3-methyl-6-((naphthalen-2-ylmethyl)thio)-1,2,3,4-tetrahydro-1,3,5-triazine

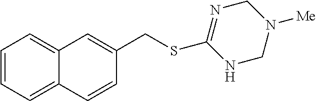 [SMILES: CN1CN=C(SCc2ccc3ccccc3c2)NC1]

### 3-Methyl-6-((naphthalen-2-ylmethyl)thio)-1,2,3,4-tetrahydro-1,3,5-triazine

Methyl amine (32 μL, 0.68 mmol) was mixed well with formaldehyde (135 μl of a 37% wt. solution in water, 1.36 mmol) in dioxane (20 ml). To this solution, naphthalen-2-ylmethyl carbamimidothioate hydrobromide (200 mg, 0.68 mmol) was added. The reaction mixture was heated until a solution was obtained and allowed to stir overnight at room temperature. The residue was purified on an ISCO chromatograph (10% methanol/methylene chloride+1% NH₄OH) to give the product as a white solid (123 mg, 67%); ¹H NMR (300 MHz) (CDCl₃) δ 7.90 (s, 1H), 7.73-7.70 (m, 4H), 7.44-7.42 (m, 3H), 4.76 (s, 2H), 4.18 (m, 4H), 2.14 (s, 3H). LC/MS RT=2.489 (M+H⁺: 272).

### Example 14. Preparation of (6-(trifluoromethyl)pyridin-3-yl)methyl Carbamimidothioate Hydrochloride

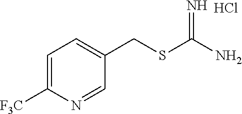 [SMILES: N=C(N)SCc1ccc(C(F)(F)F)nc1 ; Cl]

### (6-(Trifluoromethyl)pyridin-3-yl)methyl Carbamimidothioate Hydrochloride

5-(Chloromethyl)-2-(trifluoromethyl)pyridine (516 mg, 2.64 mmol) and thiourea (167 mg, 2.20 mmol) were dissolved in ethanol (20 mL). The reaction mixture was refluxed for 2 hours. The reaction mixture was cooled, and ethanol was removed under reduced pressure. The resulting residue was suspended in dichloromethane. The suspension was filtered to give product as a white solid (517 mg, 87%); ¹H NMR (300 MHz) (DMSO-d₆) δ 9.38 (s, 2H), 9.18 (s, 2H), 8.82 (s, 1H), 8.15-8.12 (d, 1H), 7.96-7.94 (d, 1H), 4.65 (s, 2H); LC/MS RT=2.194 (M+H⁺: 236).

### Example 15. Preparation of benzofuran-5-ylmethyl Carbamimidothioate Hydrobromide

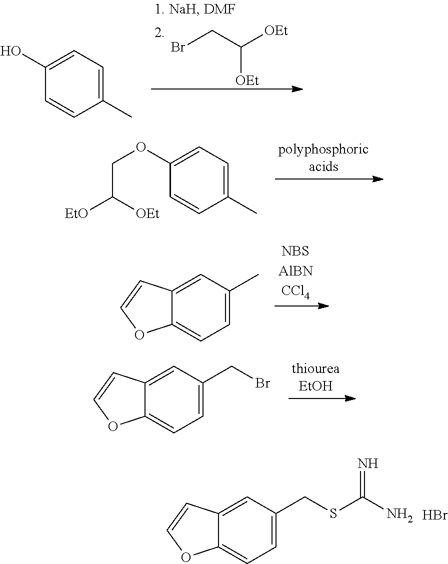

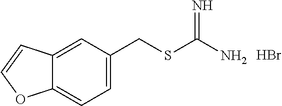 [SMILES: N=C(N)SCc1ccc2occc2c1 ; Br]

### Benzofuran-5-ylmethyl Carbamimidothioate Hydrobromide

5-(Bromomethyl)benzofuran (75 mg, 0.36 mmol) and thiourea (14 mg, 0.18 mmol) were dissolved in ethanol (5 mL). The reaction mixture was refluxed for 2 hours. The reaction mixture was cooled, and ethanol was removed under reduced pressure. The resulting residue was suspended in dichloromethane. The suspension was filtered to give a product as a white solid (52 mg, 100%); ¹H NMR (300 MHz) (DMSO-d₆) δ 9.14 (bs, 2H), 8.94 (bs, 2H), 8.02 (d, J=2 Hz, 1H), 7.69 (s, 1H), 7.60 (d, J=8 Hz, 1H), 7.34 (dd, J=9 Hz, J=2 Hz, 1H), 6.96 (d, J=1 Hz, 1H), 4.55 (s, 2H); LC/MS RT=2.37 (M+H⁺: 207).

The requisite intermediates were prepared as follows:

### Step 1)

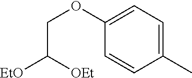 [SMILES: CCOC(COc1ccc(C)cc1)OCC]

### 1-(2,2-Diethoxyethoxy)-4-methylbenzene

4-Methylphenol (3.0 g, 27.74 mmol) was dissolved in dimethylformamide (30 mL), and it was cooled to 0° C. NaH (60% dispersed in oil, 1.22 g, 30.50 mmol) was added in portions. The reaction mixture was then stirred for 30 min at room temperature. Then, 2-bromo-1,1-diethoxyethane was added. The reaction mixture was then refluxed overnight. The reaction mixture was cooled, and it was diluted with ethyl acetate. The organic layer was washed with water followed by brine. The organic layer was dried over Na₂SO₄ and was concentrated under reduced pressure to give the product as brown oil (5.69 g, 100%); ¹H NMR (300 MHz) (CDCl₃) δ 7.07 (d, J=9 Hz, 2H), 6.82 (dd, J=6 Hz, J=2 Hz, 2H), 4.83 (t, J=5 Hz, 1H), 3.98 (d, J=5 Hz, 2H), 3.81-3.58 (m, 4H), 2.28 (s, 3H), 1.25 (t, J=7 Hz, 6H).

### Step 2)

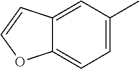

### 5-Methylbenzofuran

1-(2,2-Diethoxyethoxy)-4-methylbenzene (500 mg, 2.05 mmol) and polyphosphoric acids (239 mg, 2.05 mmol) were dissolved in toluene (10 mL). The reaction mixture was refluxed for 3 hours. The reaction mixture was cooled, and it was concentrated under reduced pressure. The resulting residue was purified on an ISCO chromatography (100% hexane) to give the product as colorless oil (107 mg, 39%); ¹H NMR (300 MHz) (CDCl₃) δ 7.63 (d, J=2 Hz, 1H), 7.45 (m, 2H), 7.16 (dd, J=8 Hz, J=Hz, 1H), 6.74 (d, J=3 Hz, 1H), 2.50 (s, 3H).

### Step 3)

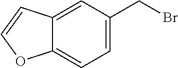 [SMILES: BrCc1ccc2occc2c1]

### 5-(Bromomethyl)benzofuran

5-Methylbenzofuran (100 mg, 0.76 mmol), N-bromosuccinimide (135 mg, 0.76 mmol) and AIBN (26 mg, 0.08 mmol) were dissolved in carbon tetrachloride (10 mL). The reaction mixture was refluxed for 3 hours. The reaction mixture was cooled, and the suspension was filtered. The filtrate was concentrated under reduced pressure. The resulting residue was purified on an ISCO chromatography (100% hexane) to give the product as colorless oil (76 mg, 78%); ¹H NMR (300 MHz) (CDCl₃) δ 7.65-7.63 (m, 2H), 7.48 (d, J=8 Hz, 1H), 7.34 (dd, J=9 Hz, J=2 Hz, 1H), 6.76-6.75 (m, 1H), 4.64 (s, 2H).

### Example 16. Preparation of Benzo[b]thiophen-5-ylmethyl Carbamimidothioate Hydrobromide

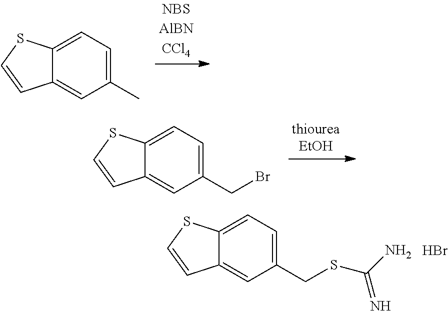

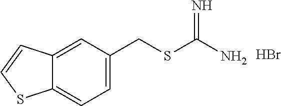 [SMILES: N=C(N)SCc1ccc2sccc2c1 ; Br]

### Benzo[b]thiophen-5-ylmethyl Carbamimidothioate Hydrobromide

5-(Bromomethyl)benzo[b]thiophene (312 mg, 1.37 mmol) and thiourea (35 mg, 0.46 mmol) were dissolved in ethanol (10 mL). The reaction mixture was refluxed for 2 hours. The reaction mixture was cooled, and ethanol was removed under reduced pressure. The resulting residue was suspended in dichloromethane. The suspension was filtered to give a product as a white solid (117 mg, 84%); ¹H NMR (300 MHz) (DMSO-d₆) δ 9.16 (bs, 2H), 8.94 (bs, 2H), 8.01 (d, J=8 Hz, 1H), 7.90 (s, 1H), 7.81 (d, J=5 Hz, 1H), 7.45 (d, J=6 Hz, 1H), 7.39 (d, J=8 Hz, 1H), 4.58 (s, 2H); LC/MS RT=2.50 (M+H⁺: 223).

The requisite intermediate was prepared as follows:

### Step 1)

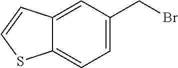 [SMILES: BrCc1ccc2sccc2c1]

### 5-(Bromomethyl)benzo[b]thiophene

5-Methylbenzo[b]thiophene (500 mg, 3.37 mmol), N-bromosuccinimide (560 mg, 3.37 mmol) and AIBN (28 mg, 0.05 mmol) were dissolved in carbon tetrachloride (45 mL). The reaction mixture was refluxed for 3 hours. The reaction mixture was cooled, and the suspension was filtered. The filtrate was concentrated under reduced pressure. The resulting residue was purified on an ISCO chromatography (100% hexane) to give the product as a white solid (312 mg, 41%); ¹H NMR (300 MHz) (CDCl₃) δ 7.87-7.85 (m, 2H), 7.48 (d, J=6 Hz, 1H), 7.39 (dd, J=8 Hz, J=2 Hz, 1H), 7.32 (dd, J=6 Hz, 1H), 4.66 (s, 2H).

### Example 17. Preparation of t-Butyl 5-((carbamimidoylthio)methyl)-1H-indole-1-carboxylate

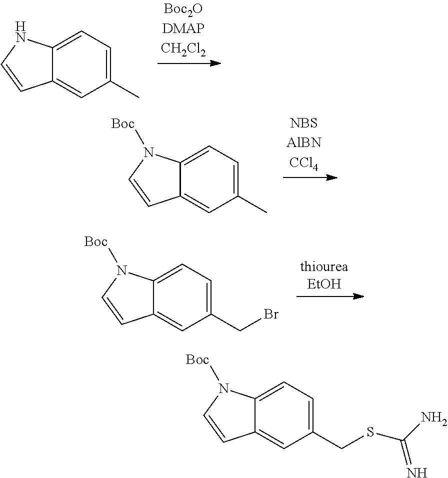

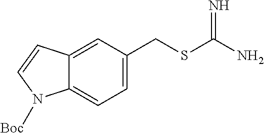 [SMILES: CC(C)(C)OC(=O)n1ccc2cc(CSC(=N)N)ccc21]

### t-Butyl 5-((carbamimidoylthio)methyl)-1H-indole-1-carboxylate

t-Butyl 5-(bromomethyl)-1H-indole-1-carboxylate (170 mg, 0.55 mmol) and thiourea (11 mg, 0.14 mmol) were dissolved in ethanol (10 mL). The reaction mixture was refluxed for 2 hours. The reaction mixture was cooled, and ethanol was removed under reduced pressure. The residue was purified on an ISCO chromatography (100% ethyl acetate followed by 10% methanol/dichloromethane+1% NH₄OH) to give the product as a red solid. (28 mg, 65%); ¹H NMR (300 MHz) (CD₃OD) δ 8.12 (d, J=9 Hz, 1H), 7.65 (d, J=4 Hz, 1H), 7.63 (s, 1H), 7.35 (dd, J=8 Hz, J=2 Hz, 1H), 6.61 (d, J=3 Hz, 1H), 4.53 (s, 2H), 1.67 (s, 9H); LC/MS RT=2.72 (M+H⁺: 306).

The requisite intermediates were prepared as follows:

### Step 1)

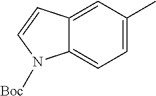 [SMILES: Cc1ccc2c(ccn2C(=O)OC(C)(C)C)c1]

### t-Butyl 5-methyl-1H-indole-1-carboxylate

5-Methyl-1H-indole (2.0 g, 15.25 mmol), di-t-butyl dicarbonate (4.99 g, 22.88 mmol) and DMAP (93 mg, 0.76 mmol) were dissolved in dichloromethane (100 mL). The reaction mixture was stirred for 6 hours at room temperature. The reaction mixture was diluted with dichloromethane, and it was washed with sat. ammonium chloride followed by brine. The organic layer was dried over Na₂SO₄ and was concentrated under reduced pressure. The residue was purified on an ISCO chromatography (0-30% ethyl acetate/hexane) to give the product as colorless oil (3.53 g, 100%); ¹H NMR (300 MHz) (CDCl₃) δ 8.02 (d, J=8 Hz, 1H), 7.56 (d, J=3 Hz, 1H), 7.35 (s, 1H), 7.14 (d, J=8 Hz, 1H), 6.50 (d, J=4 Hz, 1H), 2.45 (s, 3H), 1.68 (s, 9H).

### Step 2)

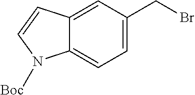 [SMILES: CC(C)(C)OC(=O)n1ccc2cc(CBr)ccc21]

### t-Butyl 5-(bromomethyl)-1H-indole-1-carboxylate

t-Butyl 5-methyl-1H-indole-1-carboxylate (750 mg, 3.24 mmol), N-bromosuccinimide (577 mg, 3.24 mmol) and AIBN (53 mg, 0.32 mmol) were dissolved in carbon tetrachloride (25 mL). The reaction mixture was refluxed for 3 hours. The reaction mixture was cooled, and the suspension was filtered. The filtrate was concentrated under reduced pressure. The resulting residue was purified on an ISCO chromatography (100% hexane) to give the product as colorless oil (170 mg, 17%); ¹H NMR (300 MHz) (CDCl₃) δ 8.12 (d, J=Hz, 1H), 7.62 (d, J=4 Hz, 1H), 7.58 (d, J=1 Hz, 1H), 7.35 (dd, J=8 Hz, J=2 Hz, 1H), 6.55 (d, J=5 Hz, 1H), 4.64 (s, 2H), 1.68 (s, 9H).

### Example 18. Preparation of quinolin-6-ylmethyl Carbamimidothioate Hydrobromide

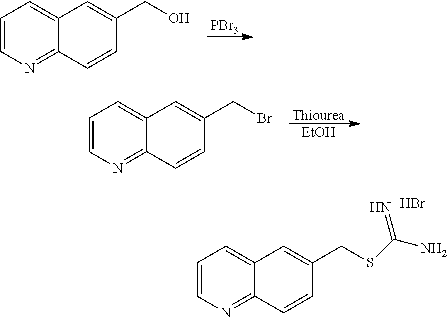

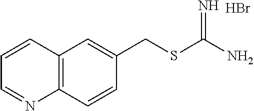 [SMILES: N=C(N)SCc1ccc2ncccc2c1 ; Br]

### Quinolin-6-ylmethyl Carbamimidothioate Hydrobromide

6-(Bromomethyl)quinoline (528 mg, 2.38 mmol) and thiourea (151 mg, 1.98 mmol) were dissolved in ethanol (20 mL). The reaction mixture was refluxed for 2 hours. The reaction mixture was cooled, and ethanol was removed under reduced pressure. The resulting residue was suspended in dichloromethane. The suspension was filtered to give a product as a brown solid (525 mg, 89%); ¹H NMR (300 MHz) (DMSO-d₆) δ 9.24 (s, 2H), 9.00 (s, 2H), 8.94-8.92 (d, 2H), 8.41-8.39 (d, 1H), 8.06-8.02 (m, 2H), 7.83-7.79 (d, 2H), 7.61-7.57 (dd, 1H), 4.70 (s, 2H).

The requisite intermediate was prepared as follows:

### Step 1)

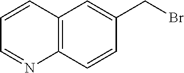 [SMILES: BrCc1ccc2ncccc2c1]

### 6-(bromomethyl)quinoline

Quinolin-6-ylmethanol (500 mg, 3.14 mmol) and phosphorus tribromide (0.33 ml, 3.46 mmol) were dissolved in dichloromethane (20 mL). The reaction mixture was stirred for overnight at room temperature. The reaction mixture was quenched with ice water. The reaction mixture was diluted with dichloromethane, and it was washed with saturated sodium bicarbonate followed by brine. The organic layer was dried over Na₂SO₄ and concentrated under reduced pressure to give the crude product as an orange solid (528 mg, 76%). The crude product was used directly without further purification; ¹H NMR (300 MHz) (CDCl₃) δ 8.93-8.91 (m, 1H), 8.16-8.09 (m, 2H), 7.83-7.82 (d, 1H), 7.77-7.73 (dd, 1H), 7.45-7.41 (q, 1H), 4.67 (s, 2H).

### Example 19. Preparation of quinolin-8-ylmethyl Carbamimidothioate Hydrobromide

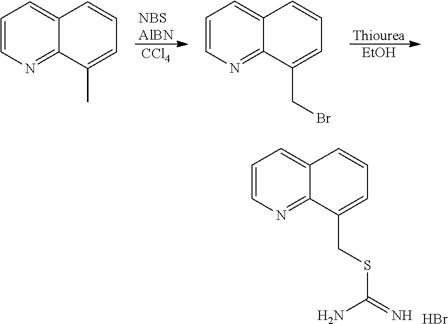

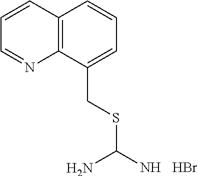 [SMILES: NC(N)SCc1cccc2cccnc12 ; Br]

### Quinolin-8-ylmethyl Carbamimidothioate Hydrobromide

8-(Bromomethyl)quinoline (500 mg, 2.25 mmol) and thiourea (143 mg, 1.98 mmol) were dissolved in ethanol (20 mL). The reaction mixture was refluxed for 2 hours. The reaction mixture was cooled, and ethanol was removed under reduced pressure. The resulting residue was suspended in dichloromethane. The suspension was filtered to give the product as a white solid (501 mg, 90%); ¹H NMR (300 MHz) (DMSO-d₆) δ 9.44 (s, 2H), 8.99-8.98 (m, 2H), 8.48-8.45 (d, 1H), 8.02-8.00 (d, 1H), 7.93-7.91 (d, 1H), 7.66-7.59 (m, 2H), 4.98 (s, 2H).

The requisite intermediate was prepared as follows:

### Step 1)

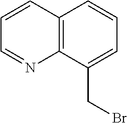 [SMILES: BrCc1cccc2cccnc12]

### 8-(bromomethyl)quinoline

8-Methylquinoline (1.00 g, 6.98 mmol) in carbon tetrachloride (20 mL) was added N-bromosuccinimide (1.37 g, 7.69 mmol) and AIBN (115 mg, 0.70 mmol). The reaction mixture was stirred at reflux for 3 hours. The reaction mixture was cooled and carbon tetrachloride was removed under reduced pressure. The residue was purified on an ISCO chromatograph (0-10% ethyl acetate/hexane) to give the product as a white solid (1.26 g, 82%); ¹H NMR (300 MHz) (CDCl₃) δ 9.03-9.01 (m, 1H), 8.18-8.15 (d, 1H), 7.85-7.79 (m, 2H), 7.54-7.43 (m, 2H). 5.25 (s, 2H).

### Example 20. Preparation of Isoquinolin-5-ylmethyl Carbamimidothioate Hydrobromide

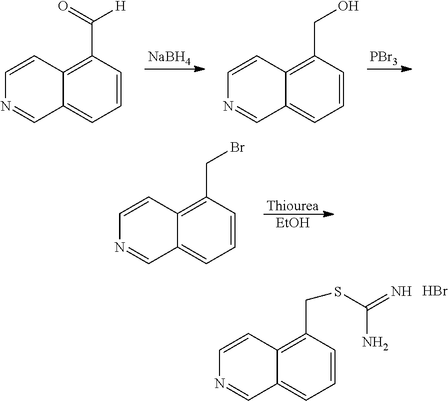

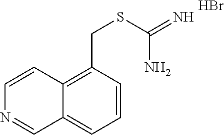 [SMILES: Br ; N=C(N)SCc1cccc2cnccc12]

### Isoquinolin-5-ylmethyl Carbamimidothioate Hydrobromide

5-(Bromomethyl)isoquinoline (254 mg, 1.14 mmol) and thiourea (73 mg, 0.95 mmol) were dissolved in ethanol (20 mL). The reaction mixture was refluxed for 2 hours. The reaction mixture was cooled, and ethanol was removed under reduced pressure. The resulting residue was suspended in dichloromethane. The suspension was filtered to give product as a brown solid (136 mg, 66%); ¹H NMR (300 MHz) (DMSO-d₆) δ 9.42 (s, 1H), 9.28 (s, 2H), 9.08 (s, 2H), 8.63-8.62 (d, 1H), 8.18-8.15 (m, 2H), 7.93-7.91 (d, 1H), 7.73-7.68 (m, 1H), 5.01 (s, 2H).

The requisite intermediates were prepared as follows:

### Step 1)

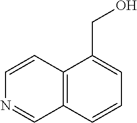 [SMILES: OCc1cccc2cnccc12]

### Isoquinolin-5-ylmethanol

Isoquinoline-5-carbaldehyde (1.00 g, 6.36 mmol) and sodium borohydride (482 mg, 12.8 mmol) were dissolved in methanol (20 mL) at 0° C. The reaction mixture was stirred from 0° C. to room temperature for overnight. The resulting mixture was then quenched with ice water. Remove methanol and perform extraction with dichloromethane and brine water. The organic layer was dried over Na₂SO₄ and concentrated under reduced pressure to give the crude product as a brown oil (957 mg, 95%). The crude product was used directly without further purification; ¹H NMR (300 MHz) (CDCl₃) δ 8.89 (s, 1H), 8.21-8.19 (d, 1H), 7.69-7.67 (d, 1H), 7.64-7.60 (m, 2H), 7.39-7.33 (m, 1H), 4.96 (s, 2H).

### Step 2)

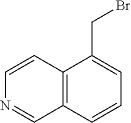 [SMILES: BrCc1cccc2cnccc12]

### 5-(bromomethyl)isoquinoline

Isoquinolin-5-ylmethanol (957 mg, 6.01 mmol) and phosphorus tribromide (0.63 ml, 6.62 mmol) were dissolved in dichloromethane (20 mL). The reaction mixture was stirred for overnight at room temperature. The reaction mixture was quenched with ice water. The reaction mixture was diluted with dichloromethane, and it was washed with saturated sodium bicarbonate followed by brine. The organic layer was dried over Na₂SO₄ and concentrated under reduced pressure to give the crude product as an orange solid (978 mg, 74%). The crude product was used directly without further purification; ¹H NMR (300 MHz) (CDCl₃) δ 9.32 (s, 1H), 8.67-8.65 (d, 1H), 8.01-7.95 (m, 2H), 7.79-7.77 (d, 1H), 7.60-7.55 (m, 1H), 4.92 (s, 2H).

### Example 21. Description of General Test Methods

### Intrinsic MIC Assays

MIC assays were conducted in accordance with Clinical and Laboratory Standards Institute (CLSI) guidelines for broth microdilution. A 96-well plate containing cation-adjusted Mueller-Hinton (CAMH broth with 2-fold serial dilution of compounds was inoculated with log-phase bacterial at 5×10⁵ CFU/mL. The final volume in each well was 100 μL. Each compound was tested in duplicate. The microtiter plates were incubated in an aerobic environment for 18 hours at 37° C. Then the bacterial growth was tested by reading the plate with a VersaMax plate reader (Molecular Devices, Inc.) at 600 nm. The MIC was defined as the lowest compound concentration that inhibited 90% of bacteria growth.

The intrinsic MIC of the experimental EPIs was tested with the method described. The 2-fold serial dilution begins with 100 μg/mL of tested compound in the first column of the 96-well plates. The following Gram-negative bacterial strains were included in these assays:

Escherichia coli ATCC 25922

Klebsiella pneumoniae ATCC 13883 and ATCC 10031

Pseudomonas aeruginosa ATCC 27853.

Pseudomonas aeruginosa PAO1

Acinetobacter baumannii ATCC 19606

### MIC Assays in the Presence of a Bacterial Efflux Inhibitor

The EPI assay for the purposes of these studies represents a MIC assay in which the MIC of the antibiotic against the bacteria is tested in the presence of an experimental efflux pump inhibitor (EPI). The highest concentration of the EPI present in the assay typically is ½ of the intrinsic MIC of the compound. If the intrinsic MIC of the EPI is greater than 100 μg/mL, the EPI assay was tested with 50 μg/mL. Using serial dilutions of the EPI, its enhancement of antibiotic activity was then evaluated. The relative EPI activity was decided by comparing the MIC of the antibiotic in the presence of the EPI compound with the intrinsic MIC of the antibiotic alone. For comparative purposes, we generally used EPIs at concentration of 12.5 and 6.25 μg/ml against varying concentration of our test antibiotic.

### Example 22

The following can illustrate representative pharmaceutical dosage forms, containing a compound of formula I, II, III, IV or V (‘Compound X’) or a pharmaceutically acceptable salt thereof, for therapeutic or prophylactic use in humans. The tablets can optionally comprise an enteric coating.

| | mg/tablet |
|---|---|
| (i) Tablet 1 | |
| Compound X= | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |
| (ii) Tablet 2 | |
| Compound X= | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | ms/capsule |
|---|---|
| Compound X= | 10.0 |
| Colloidal silicon dioxide | 1.5 |
| Lactose | 465.5 |
| Pregelatinized starch | 120.0 |
| Magnesium stearate | 3.0 |
| | 600.0 |

| | mg/ml |
|---|---|
| (iv) Injection 1 (1 mg/ml) | |
| Compound X= (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | |
| (pH adjustment to 7.0-7.5) | q.s. |
| Water for injection | q.s. ad 1 mL |
| (v) Injection 2 (10 mg/ml) | |
| Compound X= (free acid form) | 10.0 |
| Monobasic sodium phosphate | 0.3 |
| Dibasic sodium phosphate | 1.1 |
| Polyethylene glycol 400 | 200.0 |
| 1.0N Sodium hydroxide solution | |
| (pH adjustment to 7.0-7.5) | q.s. |
| Water for injection | q.s. ad 1 mL |

| (vi) Aerosol | mg/can |
|---|---|
| Compound X= | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The above formulations may be obtained by conventional procedures well known in the pharmaceutical art.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A method of treating or preventing a bacterial infection in an animal comprising administering to the animal a bacterial efflux pump inhibitor and a compound of formula I: 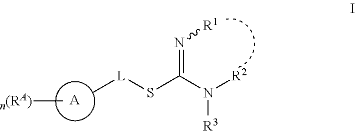
or a pharmaceutically acceptable salt thereof, wherein:
R¹ is hydrogen, (C₁-C₄)alkyl, or (C₁-C₄)haloalkyl, and R² is hydrogen, (C₁-C₄)alkyl, or (C₁-C₄)haloalkyl; or R¹ and R² taken together with the atoms to which they are attached form a tetrahydro-1,3,5-triazinyl which is optionally substituted with one or more R⁴ groups;
R³ is hydrogen, (C₁-C₄)alkyl, or (C₁-C₄)haloalkyl;
each R⁴ is independently hydrogen, halo, hydroxyl, nitro, cyano, (C₁-C₆)alkyl, aryl or heteroaryl, wherein the (C₁-C₆)alkyl is optionally substituted with one or more groups selected from halo, hydroxy, nitro, cyano, (C₁-C₄)alkoxy, —NR^{X}R^{Y}, aryl, heteroaryl, aryloxy, or heteroaryloxy, wherein any ary, heteroaryl, aryloxy and heteroaryloxy is optionally substituted with one or more groups selected from halo, hydroxy, (C₁-C₄)alkyl, (C₁-C₄)haloalkyl, (C₁-C₄)alkoxy, nitro, cyano, or —NR^{X}R^{Y};
L is (C₁-C₅)alkylene that is optionally substituted with one or more R^{L} groups;
each R^{L} is independently selected from hydrogen, halo, hydroxy, nitro, cyano, or (C₁-C₄)alkoxy; or any two R^{L} groups that are attached to the same carbon taken together form a (C₃-C₆)carbocycle;
A is aryl or heteroaryl;
each R^{A} is independently selected from the group consisting of halo, cyano, nitro, (C₁-C₆)alkyl, (C₁-C₆)alkoxy, —NR^{X}R^{Y}, aryl or heteroaryl; wherein the (C₁-C₆)alkyl and (C₁-C₆)alkoxy is optionally substituted with one or more groups selected from oxo, halo, hydroxy, (C₁-C₄)alkoxy, nitro, cyano, or —NR^{X}R^{Y}; wherein the aryl and heteroaryl is optionally substituted with one or more groups selected from halo, hydroxy, (C₁-C₄)alkyl, (C₁-C₄)haloalkyl, (C₁-C₄)alkoxy, nitro, cyano, or —NR^{X}R^{Y};
each R^{X} and R^{Y} are independently hydrogen or (C₁-C₄)alkyl; or R^{X} and R^{Y} taken together with the nitrogen to which they are attached form pyrrolidinyl, piperidinyl, piperazinyl, or morpholinyl; and
n is 0, 1, 2, 3, or 4.

2. The method of claim 1 wherein the bacterial efflux pump inhibitor is a compound of formula II: 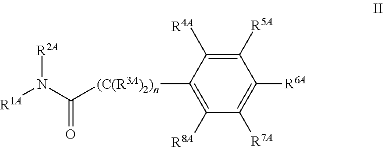
or a pharmaceutically acceptable salt thereof, wherein:
R^{1A} is (C₃-C₈)alkyl substituted with two or more groups selected from —NR^{b1}R^{c1}, —NHNH₂, —C(═NR^{a1})(NR^{b1}R^{c1}), —NR^{a1}C(═NR^{a1})(R^{d1}) and —NR^{a1}C(═NR^{a1})(NR^{b1}R^{c1});
R^{2A} is hydrogen or (C₁-C₃)alkyl;
each R^{3A} is independently hydrogen, halo or (C₁-C₄)alkyl;
R^{4A} is hydrogen, halo, (C₁-C₆)alkyl, (C₁-C₆)haloalkyl, (C₁-C₆)alkoxy, (C₁-C₄)haloalkoxy, aryl or heteroaryl wherein the aryl or heteroaryl is optionally substituted with one or more groups independently selected from halo, (C₁-C₄)alkyl, (C₁-C₄)haloalkyl, (C₁-C₄)alkoxy and (C₁-C₄)haloalkoxy;
R^{5A} is hydrogen, halo, (C₁-C₆)alkyl, (C₁-C₆)haloalkyl, (C₁-C₆)alkoxy, (C₁-C₄)haloalkoxy, aryl or heteroaryl wherein the aryl or heteroaryl is optionally substituted with one or more groups independently selected from halo, (C₁-C₄)alkyl, (C₁-C₄)haloalkyl, (C₁-C₄)alkoxy and (C₁-C₄)haloalkoxy;
R^{6A} is hydrogen, halo, (C₁-C₆)alkyl, (C₁-C₆)haloalkyl, (C₁-C₆)alkoxy, (C₁-C₄)haloalkoxy, aryl or heteroaryl wherein the aryl or heteroaryl is optionally substituted with one or more groups independently selected from halo, (C₁-C₄)alkyl, (C₁-C₄)haloalkyl, (C₁-C₄)alkoxy and (C₁-C₄)haloalkoxy;
R^{7A} is hydrogen, halo, (C₁-C₆)alkyl, (C₁-C₆)haloalkyl, (C₁-C₆)alkoxy, (C₁-C₄)haloalkoxy, aryl or heteroaryl wherein the aryl or heteroaryl is optionally substituted with one or more groups independently selected from halo, (C₁-C₄)alkyl, (C₁-C₄)haloalkyl, (C₁-C₄)alkoxy and (C₁-C₄)haloalkoxy;
R^{8A} is hydrogen, halo, (C₁-C₆)alkyl, (C₁-C₆)haloalkyl, (C₁-C₆)alkoxy, (C₁-C₄)haloalkoxy, aryl or heteroaryl wherein the aryl or heteroaryl is optionally substituted with one or more groups independently selected from halo, (C₁-C₄)alkyl, (C₁-C₄)haloalkyl, (C₁-C₄)alkoxy and (C₁-C₄)haloalkoxy;
each R^{a1} is independently hydrogen or (C₁-C₄)alkyl;
each R^{b1} and R^{C1} is independently hydrogen or (C₁-C₄)alkyl;
R^{d1} is (C₁-C₃)alkyl and
n is 0 or 1.

3. The method of claim 1 wherein the bacterial efflux pump inhibitor is a compound of formula III: 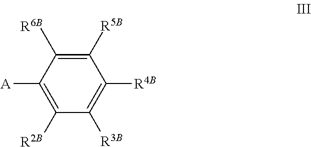
or a pharmaceutically acceptable salt thereof, wherein:
A is —C(═O)N(R^{a1})—R^{1B}, —(C₁-C₃)alkyl-C(═O)N(R^{a1})R^{1B}, —(C₁-C₃)alkyl-O—R^{1B}, —O—R^{1B}, —(C₁-C₃)alkyl-N(R^{a1})—R^{1B}, or —N(R^{a1})—R^{1B};
each R^{1B} is independently a (C₃-C₇)carbocyclyl, (C₃-C₇)carbocyclyl-(C₁-C₄)alkyl-, 4-7 membered monocyclic heterocyclyl, or 4-7 membered monocyclic heterocyclyl-(C₁-C₄)alkyl-, wherein each (C₃-C₇)carbocyclyl or (C₃-C₇)carbocyclyl-(C₁-C₄)alkyl- is independently substituted with one or more groups selected from the group consisting of Z and —(C₁-C₆)alkyl substituted with one or more Z, and wherein each 4-7 membered monocyclic heterocyclyl or 4-7 membered monocyclic heterocyclyl-(C₁-C₄)alkyl- is independently optionally substituted with one or more groups selected from the group consisting of Z and —(C₁-C₆)alkyl substituted with one or more Z, wherein each Z is independently selected from the group consisting of NR^{b2}R^{c2}, —NHNH₂, —C(═NR^{a2})(R^{b2}R^{c2}), —NR^{a2}C(═NR^{a2})(R^{d2}), and —NR^{a2}C(═NR^{a2})(NR^{b2}R^{c2}) and wherein each (C₃-C₇)carbocyclyl, (C₃-C₇)carbocyclyl-(C₁-C₄)alkyl-, 4-7 membered monocyclic heterocyclyl, or 4-7 membered monocyclic heterocyclyl-(C₁-C₄)alkyl-, is independently optionally substituted independently with one or more (C₁-C₄)alkyl;
R^{2B} is hydrogen, halo, (C₁-C₄)alkyl, (C₁-C₄)haloalkyl, (C₁-C₄)alkoxy, (C₁-C₄)haloalkoxy, aryl or heteroaryl wherein the aryl or heteroaryl is optionally substituted with one or more groups independently selected from halo, —NO₂, —CN, (C₁-C₄)alkyl, (C₁-C₄)haloalkyl, (C₁-C₄)alkoxy and (C₁-C₄)haloalkoxy;
R^{3B} is hydrogen, halo, (C₁-C₆)alkyl, (C₁-C₄)haloalkyl, (C₁-C₆)alkoxy, (C₁-C₄)haloalkoxy, aryl or heteroaryl wherein the aryl or heteroaryl is optionally substituted with one or more groups independently selected from halo, —NO₂, —CN, (C₁-C₆)alkyl, (C₁-C₄)haloalkyl, (C₁-C₆)alkoxy and (C₁-C₄)haloalkoxy;
R^{4B} is hydrogen, halo, (C₁-C₆)alkyl, (C₁-C₄)haloalkyl, (C₁-C₆)alkoxy, (C₁-C₄)haloalkoxy, aryl or heteroaryl wherein the aryl or heteroaryl is optionally substituted with one or more groups independently selected from halo, —NO₂, —CN, (C₁-C₆)alkyl, (C₁-C₄)haloalkyl, (C₁-C₆)alkoxy and (C₁-C₄)haloalkoxy;
R^{5B} is hydrogen, halo, (C₁-C₆)alkyl, (C₁-C₄)haloalkyl, (C₁-C₆)alkoxy, (C₁-C₄)haloalkoxy, aryl or heteroaryl wherein the aryl or heteroaryl is optionally substituted with one or more groups independently selected from halo, —NO₂, —CN, (C₁-C₆)alkyl, (C₁-C₄)haloalkyl, (C₁-C₆)alkoxy and (C₁-C₄)haloalkoxy;
R^{6B} is hydrogen, halo, (C₁-C₄)alkyl, (C₁-C₄)haloalkyl, (C₁-C₄)alkoxy, (C₁-C₄)haloalkoxy, aryl or heteroaryl wherein the aryl or heteroaryl is optionally substituted with one or more groups independently selected from halo, —NO₂, —CN, (C₁-C₄)alkyl, (C₁-C₄)haloalkyl, (C₁-C₄)alkoxy and C₁-C₄)haloalkoxy;
each R^{a1} is independently hydrogen, (C₁-C₆)alkyl or (C₃-C₇)carbocyclyl;
each R^{a2} is independently hydrogen, (C₁-C₆)alkyl or (C₃-C₇)carbocyclyl;
each R^{b2} and R^{c2} is independently hydrogen, (C₁-C₆)alkyl or (C₃-C₇)carbocyclyl; and
R^{d2} is (C₁-C₆)alkyl or (C₃-C₇)carbocyclyl.

4. The method of claim 1 wherein the bacterial efflux pump inhibitor is a compound of formula IV: 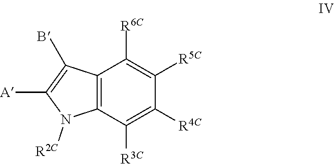
or a pharmaceutically acceptable salt thereof, wherein:
one of A′ or B′ is —C(═O)N(R^{a1})—R^{1C}, —(C₁-C₃)alkyl-C(═O)N(R^{a1})R^{1C}, —(C₁-C₃)alkyl-O—R^{1C}, —O—R^{1C}, —(C₁-C₃)alkyl-N(R^{a1})—R^{1C}, —N(R^{a1})—R^{1C}, or R^{1C} and the other of A′ or B′ is H, halogen, or (C₁-C₄)alkyl;
each R^{1C} is independently:
(a) (C₁-C₁₄)alkyl substituted with one or more groups selected from the group consisting of —NR^{b2}R^{c2}, —NHNH₂, —C(═NR^{a2})(NR^{b2}R^{c2}), —NR^{a2}C(═NR^{a2})(R^{d2}), and —NR^{a2}C(═NR^{a2})(NR^{b2}R^{c2}); and wherein (C₁-C₁₄)alkyl is optionally substituted independently with one or more halo, (C₁-C₄)alkyl or (C₃-C₇)carbocyclyl; or
(b) (C₃-C₇)carbocyclyl, (C₃-C₇)carbocyclyl-(C₁-C₄)alkyl-, 4-7 membered monocyclic heterocyclyl, 4-7 membered monocyclic heterocyclyl-(C₁-C₄)alkyl-, (C₃-C₇)carbocyclyl-NR^{e}—(C₁-C₄)alkyl- or 4-7 membered monocyclic heterocyclyl-NR^{e}—(C₁-C₄)alkyl- wherein each (C₃-C₇)carbocyclyl, (C₃-C₇)carbocyclyl-(C₁-C₄)alkyl- or —(C₃-C₇)carbocyclyl-NR^{e}—(C₁-C₄)alkyl- is independently substituted with one or more Z¹ or Z², and wherein each 4-7 membered monocyclic heterocyclyl, 4-7 membered monocyclic heterocyclyl-(C₁-C₄)alkyl- or 4-7 membered monocyclic heterocyclyl-NR^{e}—(C₁-C₄)alkyl- is independently optionally substituted with one or more Z¹ or Z², and wherein any (C₃-C₇)carbocyclyl, (C₃-C₇)carbocyclyl-(C₁-C₄)alkyl-, 4-7 membered monocyclic heterocyclyl, 4-7 membered monocyclic heterocyclyl-(C₁-C₄)alkyl-, (C₃-C₇)carbocyclyl NR^{e}—(C₁-C₄)alkyl- or 4-7 membered monocyclic heterocyclyl-NR^{e}—(C₁-C₄)alkyl- of R¹ is independently optionally substituted with one or more halo, (C₁-C₄)alkyl, (C₃-C₇)carbocyclyl, —C(═O)NH₂, —C(═O)NH(C₁-C₄)alkyl, —C(═O)N((C₁-C₄)alkyl)₂, —NHC(═O)(C₁-C₄)alkyl-NH₂, or 3-7 membered monocyclic heterocyclyl wherein (C₁-C₄)alkyl, (C₃-C₇)carbocyclyl or 3-7 membered monocyclic heterocyclyl is optionally substituted with one or more halogen, (C₁-C₄)alkyl, —NH₂, —NH(C₁-C₄)alkyl or —N((C₁-C₄)alkyl)₂;
R^{2C} is hydrogen, (C₁-C₄)alkyl or phenyl(C₁-C₃)alkyl-, wherein the phenyl is optionally substituted with one or more (C₁-C₄)alkyl, —O(C₁-C₄)alkyl, halogen, or —NO₂;
R^{3C} is hydrogen, halo, (C₁-C₄)alkyl, (C₁-C₄)haloalkyl, (C₁-C₄)alkoxy, (C₁-C₄)haloalkoxy, aryl, or heteroaryl wherein the aryl or heteroaryl is optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy;
R^{4C} is hydrogen, halo, (C₁-C₄)alkyl, (C₁-C₄)haloalkyl, (C₁-C₄)alkoxy, (C₁-C₄)haloalkoxy, aryl, heteroaryl, aryl(C₁-C₄)alkyl-, heteroaryl(C₁-C₄)alkyl-, (C₃-C₇)carbocyclyl(C₁-C₄)alkyl-, (C₃-C₇)carbocyclyl(C₂-C₄)alkynyl-, phenoxy or heteroaryloxy, wherein the aryl, heteroaryl, aryl(C₁-C₄)alkyl-, heteroaryl(C₁-C₄)alkyl-, (C₃-C₇)carbocyclyl(C₁-C₄)alkyl-, (C₃-C₇)carbocyclyl(C₂-C₄)alkynyl-, phenoxy or heteroaryloxy, is optionally substituted with one or more groups independently selected from the group consisting of halo, —OH, —NO₂, —CN, (C₁-C₄)alkyl, (C₁-C₄)haloalkyl, (C₁-C₄)alkoxy, (C₁-C₄)haloalkoxy, methylenedioxy (—OCH₂O—), and (C₃-C₇)carbocyclyl; R^{5C} is hydrogen, halo, (C₁-C₄)alkyl, (C₁-C₄)haloalkyl, (C₁-C₄)alkoxy, (C₁-C₄)haloalkoxy, aryl, heteroaryl aryl(C₁-C₄)alkyl-, heteroaryl(C₁-C₄)alkyl-, (C₃-C₇)carbocyclyl(C₁-C₄)alkyl-, (C₃-C₇)carbocyclyl(C₂-C₄)alkynyl-, phenoxy or heteroaryloxy, wherein the aryl, heteroaryl, aryl(C₁-C₄)alkyl-, heteroaryl(C₁-C₄)alkyl-, (C₃-C₇)carbocyclyl(C₁-C₄)alkyl-, (C₃-C₇)carbocyclyl(C₂-C₄)alkynyl-, phenoxy or heteroaryloxy, is optionally substituted with one or more groups independently selected from the group consisting of halo, —OH, —NO₂, —CN, (C₁-C₄)alkyl, (C₁-C₄)haloalkyl, (C₁-C₄)alkoxy, (C₁-C₄)haloalkoxy, methylenedioxy (—OCH₂O—), and (C₃-C₇)carbocyclyl;
R^{6C} is hydrogen, halo, (C₁-C₄)alkyl, (C₁-C₄)haloalkyl, (C₁-C₄)alkoxy, (C₁-C₄)haloalkoxy, aryl, or heteroaryl wherein the aryl or heteroaryl is optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy;
each Z¹ is independently selected from the group consisting of —NR^{b3}R^{c3}, —NHNH₂, —C(═NR^{a3})(NR^{b3}R^{c3}), —NR^{a3}C(═NR^{a3})(R^{d3}), and —NR^{a3}C(═NR^{a3})(NR^{b3}R^{c3});
each Z² is independently —(C₁-C₆)alkyl substituted with one or more Z¹ and optionally substituted with one or more Z³;
each Z³ is independently halo or (C₃-C₇)carbocyclyl;
each R^{a1} is independently hydrogen, (C₁-C₄)alkyl, (C₃-C₇)carbocyclyl or 3-7 membered monocyclic heterocyclyl optionally substituted with one or more halogen or (C₁-C₄)alkyl;
each R^{a2} is independently hydrogen, (C₁-C₄)alkyl or (C₃-C₇)carbocyclyl;
each R^{b2} and R^{c2} is independently hydrogen, (C₁-C₄)alkyl or (C₃-C₇)carbocyclyl;
R^{d2} is (C₁-C₄)alkyl or (C₃-C₇)carbocyclyl;
each R^{a3} is independently hydrogen (C₁-C₄)alkyl or (C₃-C₇)carbocyclyl;
each R^{b3} and R³ is independently hydrogen (C₁-C₄)alkyl or (C₃-C₇)carbocyclyl;
R^{d3} is (C₁-C₄)alkyl or (C₃-C₇)carbocyclyl; and
each R^{e} is independently hydrogen, (C₁-C₄)alkyl or (C₃-C₇)carbocyclyl.

5. The method of claim 1 wherein the bacterial efflux pump inhibitor is a compound of formula V: 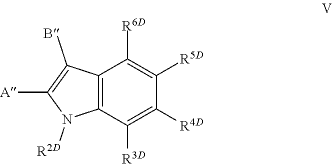
or a pharmaceutically acceptable salt thereof, wherein:
A″ is —C(═O)N(R^{a1})—R^{1D}, —(C₁-C₃)alkyl-C(═O)N(R^{a1})R^{1D}, —(C₁-C₃)alkyl-O—R^{1D}, —O—R^{1D}, —(C₁-C₃)alkyl-N(R^{a1})—R^{1D}, —N(R^{a1})—R^{1D}, or R^{1D};
B″ is (C₂-C₈)alkenyl, (C₂-C₈)alkynyl, (C₁-C₄)haloalkyl, (C₁-C₄)alkoxy, (C₁-C₄)haloalkoxy, (C₃-C₇)carbocyclyl, (C₃-C₇)carbocyclyl-(C₁-C₄)alkyl-, aryl, aryl-(C₁-C₄)alkyl-, heteroaryl, heteroaryl-(C₁-C₄)alkyl-, 3-7 membered-monocyclic-heterocycle, or 3-7 membered-monocyclic-heterocycle-(C₁-C₄)alkyl- wherein any (C₃-C₇)carbocyclyl, (C₃-C₇)carbocyclyl-(C₁-C₄)alkyl-, aryl, aryl-(C₁-C₄)alkyl-, heteroaryl, heteroaryl-(C₁-C₄)alkyl-, 3-7 membered-monocyclic-heterocycle, or 3-7 membered-monocyclic-heterocycle-(C₁-C₄)alkyl- of B″ is optionally substituted with one or more Z¹ groups;
each R^{1D} is independently:
(a) (C₁-C₁₄)alkyl substituted with one or more groups selected from the group consisting of —NR^{b2}R^{c2}, —NHN₂, —C(═NR^{a2})(NR^{b2}R^{c2}), —NR^{a2}C(═NR^{a2})(R^{d2}), and —NR^{a2}C(═NR^{a2})(NR^{b2}R^{c2}) and wherein (C₁-C₁₄)alkyl is optionally substituted independently with one or more halo, (C₁-C₄)alkyl or (C₃-C₇)carbocyclyl; or
(b) (C₃-C₇)carbocyclyl, (C₃-C₇)carbocyclyl-(C₁-C₄)alkyl-, 4-7 membered monocyclic heterocyclyl, or 4-7 membered monocyclic heterocyclyl-(C₁-C₄)alkyl-, wherein each (C₃-C₇)carbocyclyl or (C₃-C₇)carbocyclyl-(C₁-C₄)alkyl- is independently substituted with one or more Z² or Z³, and wherein each 4-7 membered monocyclic heterocyclyl or 4-7 membered monocyclic heterocyclyl-(C₁-C₄)alkyl- is independently optionally substituted with one or more Z² or Z³, and wherein any (C₃-C₇)carbocyclyl, (C₃-C₇)carbocyclyl-(C₁-C₄)alkyl-, 4-7 membered monocyclic heterocyclyl, or 4-7 membered monocyclic heterocyclyl-(C₁-C₄)alkyl- of R¹ is optionally substituted independently with one or more halo, (C₁-C₄)alkyl or (C₃-C₇)carbocyclyl;
R^{2D} is hydrogen, (C₁-C₄)alkyl or phenyl(C₁-C₃)alkyl-, wherein the phenyl is optionally substituted with one or more (C₁-C₄)alkyl, —O(C₁-C₄)alkyl, halogen, or —NO₂;
R^{3D} is hydrogen, halo, (C₁-C₄)alkyl, (C₁-C₄)haloalkyl, (C₁-C₄)alkoxy, (C₁-C₄)haloalkoxy, aryl, or heteroaryl wherein the aryl or heteroaryl is optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy;
R^{4D} is hydrogen, halo, (C₁-C₄)alkyl, (C₁-C₄)haloalkyl, (C₁-C₄)alkoxy, (C₁-C₄)haloalkoxy, aryl, or heteroaryl wherein the aryl or heteroaryl is optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy;
R^{5D} is hydrogen, halo, (C₁-C₄)alkyl, (C₁-C₄)haloalkyl, (C₁-C₄)alkoxy, (C₁-C₄)haloalkoxy, aryl, or heteroaryl wherein the aryl or heteroaryl is optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy;
R^{6D} is hydrogen, halo, (C₁-C₄)alkyl, (C₁-C₄)haloalkyl, (C₁-C₄)alkoxy, (C₁-C₄)haloalkoxy, aryl, or heteroaryl wherein the aryl or heteroaryl is optionally substituted with one or more groups independently selected from the group consisting of halo, —OH, —NO₂, —CN, (C₁-C₄)alkyl, (C₁-C₄)haloalkyl, (C₁-C₄)alkoxy, and (C₁-C₄)haloalkoxy;
each Z¹ is independently halo, —OH, —NO₂, —CN, (C₁-C₄)alkyl, (C₁-C₄)haloalkyl, (C₁-C₄)alkoxy, or (C₁-C₄)haloalkoxy;
each Z² is independently selected from the group consisting of —NR^{b3}R^{c3}, —NHNH₂, —C(═NR^{a3})(NR^{b3}R^{d3}), —NR^{a3}C(═NR^{a3})(R^{d3}), and —NR^{a3}C(═NR^{a3})(NR^{b3}R^{c3})
each Z³ is independently —(C₁-C₆)alkyl substituted with one or more Z² and optionally substituted with one or more Z⁴;
each Z⁴ is independently halo or (C₃-C₇)carbocyclyl;
each R^{a1} is independently hydrogen, (C₁-C₄)alkyl or (C₃-C₇)carbocyclyl;
each R^{a2} is independently hydrogen, (C₁-C₄)alkyl or (C₃-C₇)carbocyclyl;
each R^{b2} and R^{c2} is independently hydrogen, (C₁-C₄)alkyl or (C₃-C₇)carbocyclyl;
R^{d2} is (C₁-C₄)alkyl or (C₃-C₇)carbocyclyl;
each R^{a3} is independently hydrogen (C₁-C₄)alkyl or (C₃-C₇)carbocyclyl;
each R^{b3} and R³ is independently hydrogen (C₁-C₄)alkyl or (C₃-C₇)carbocyclyl; and
R^{d3} is (C₁-C₄)alkyl or (C₃-C₇)carbocyclyl.

6. The method of claim 1 wherein the bacterial efflux pump inhibitor is selected from the group consisting of: 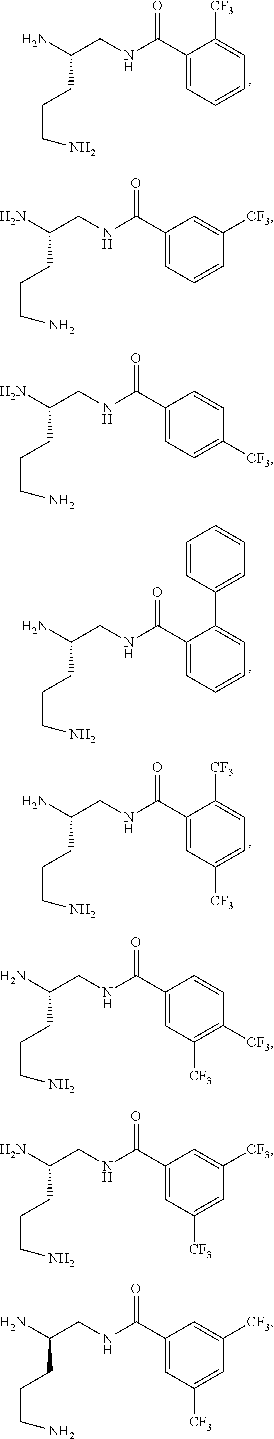 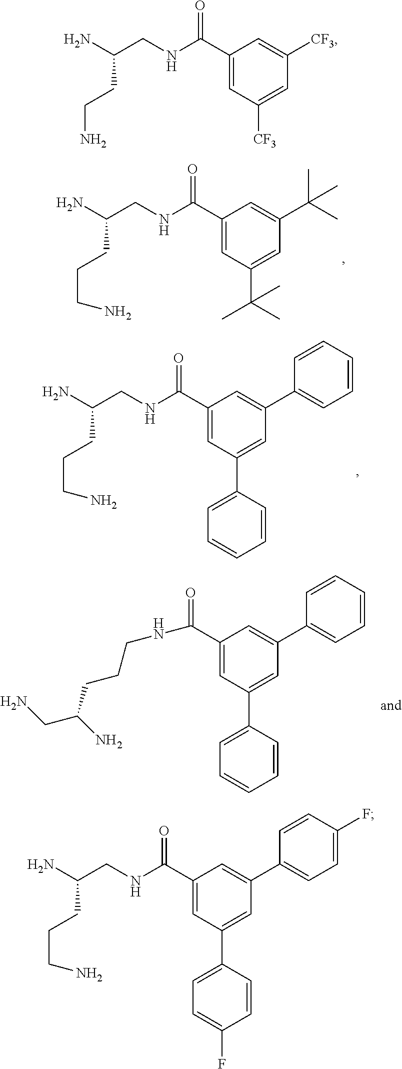
or a pharmaceutically acceptable salt thereof.

7. The method of claim 1 wherein the bacterial efflux pump inhibitor is selected from the group consisting of: 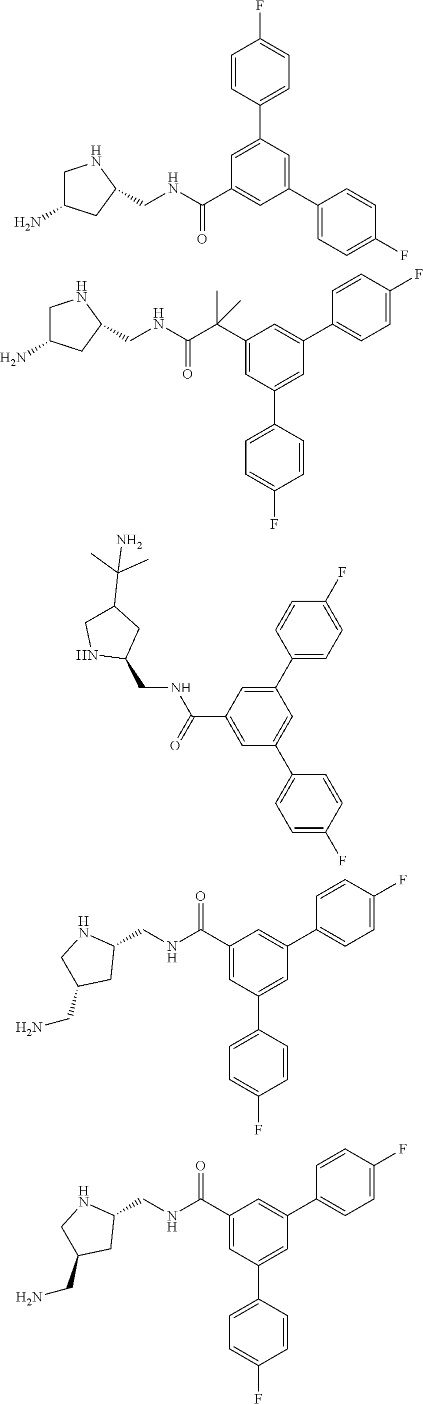 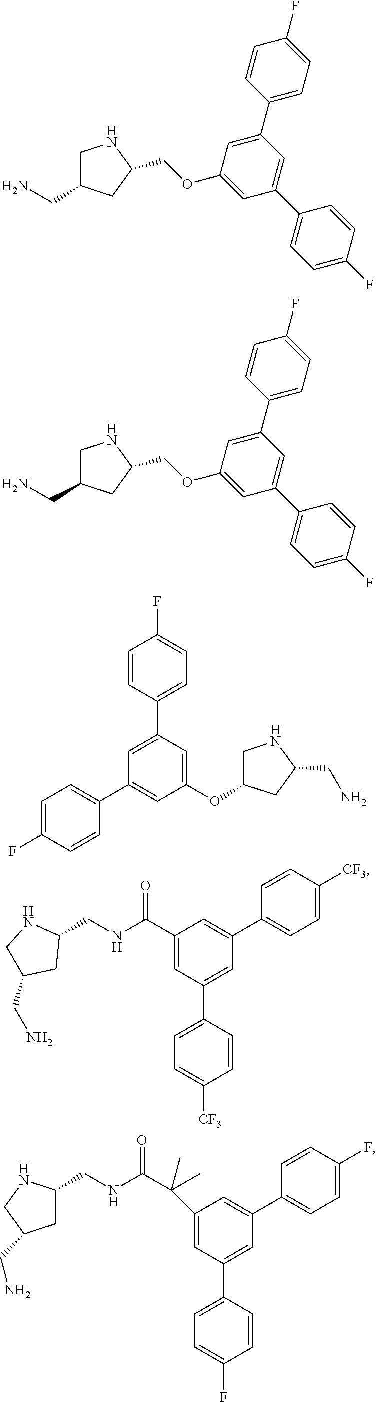 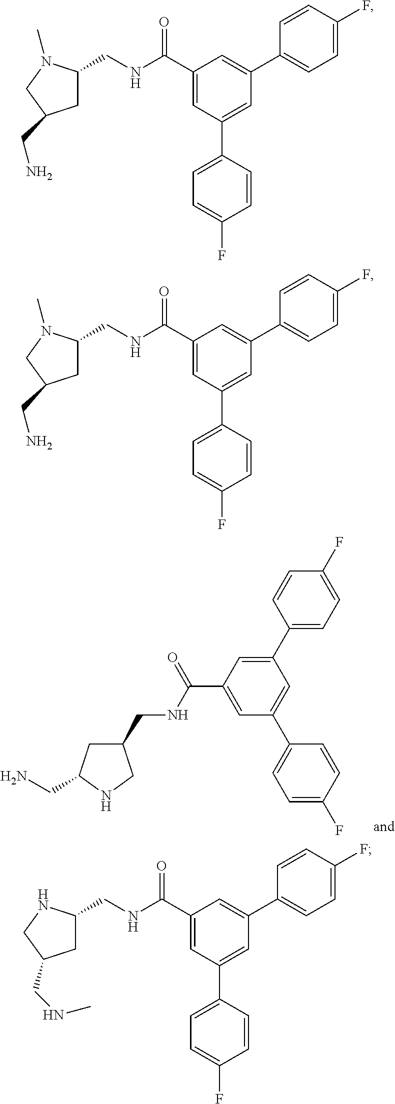
or a pharmaceutically acceptable salt thereof.

8. The method of claim 1 wherein the bacterial efflux pump inhibitor is selected from the group consisting of: 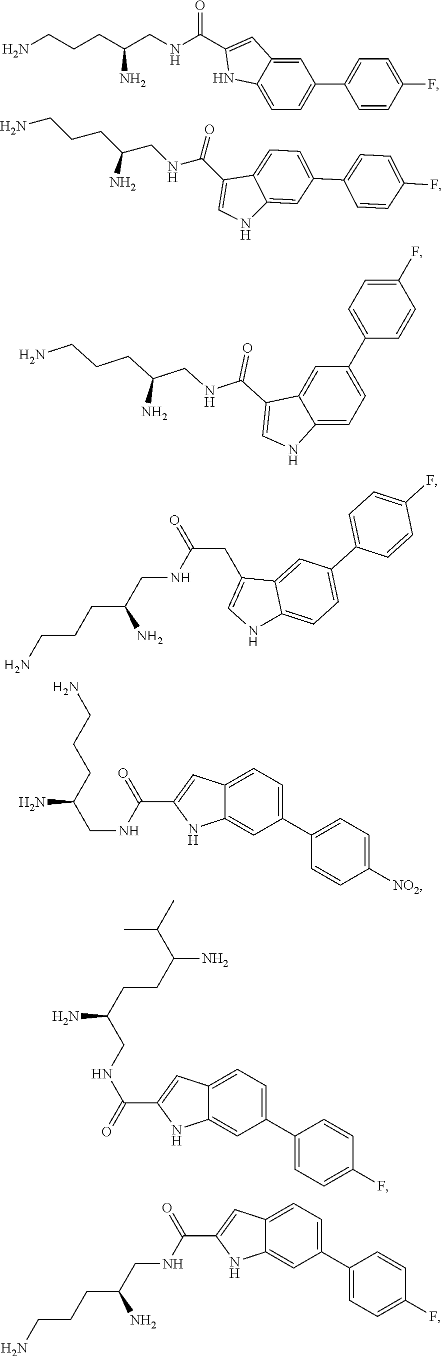 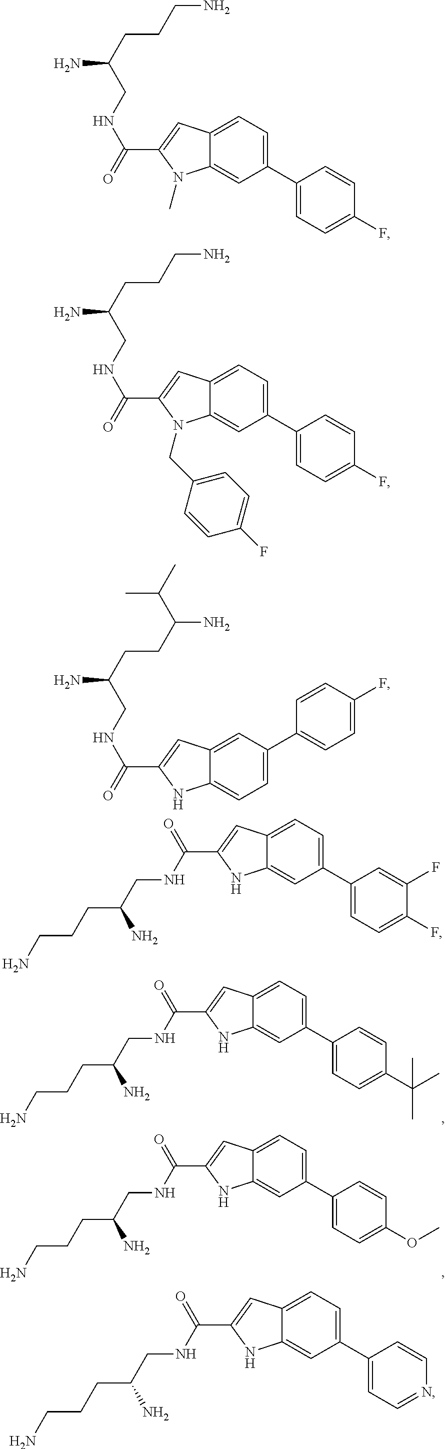 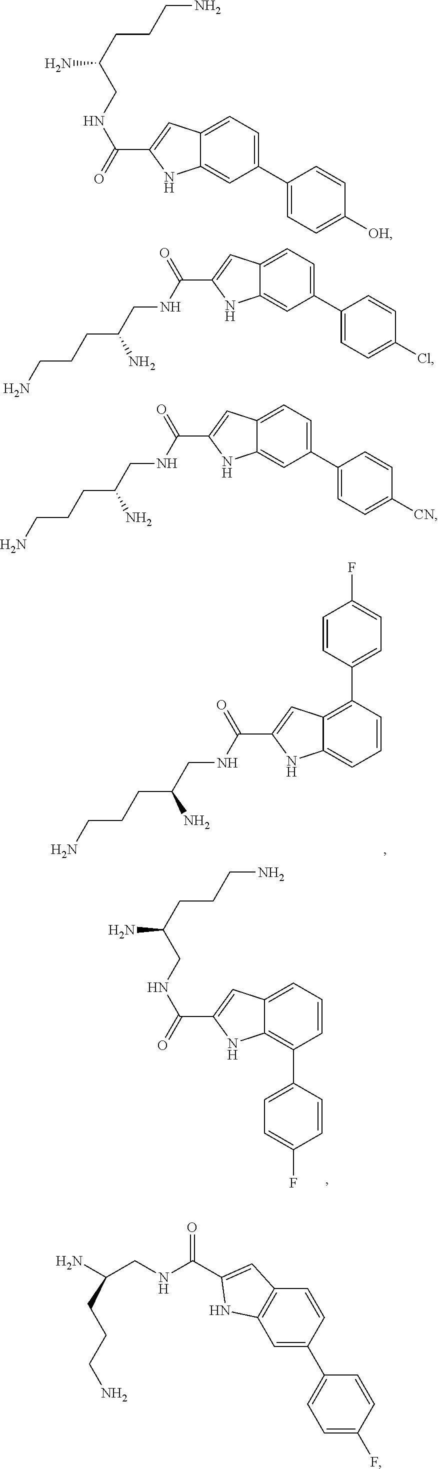 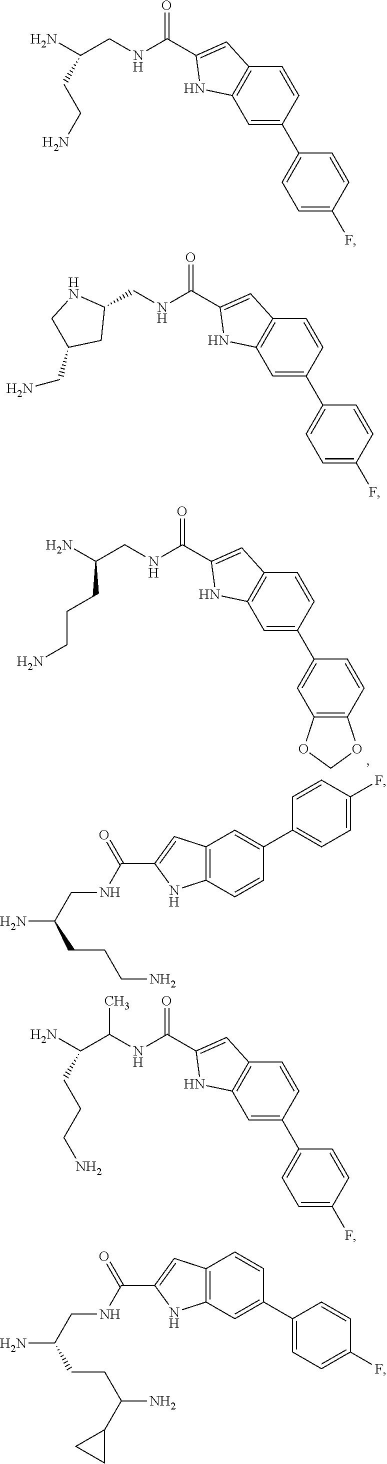 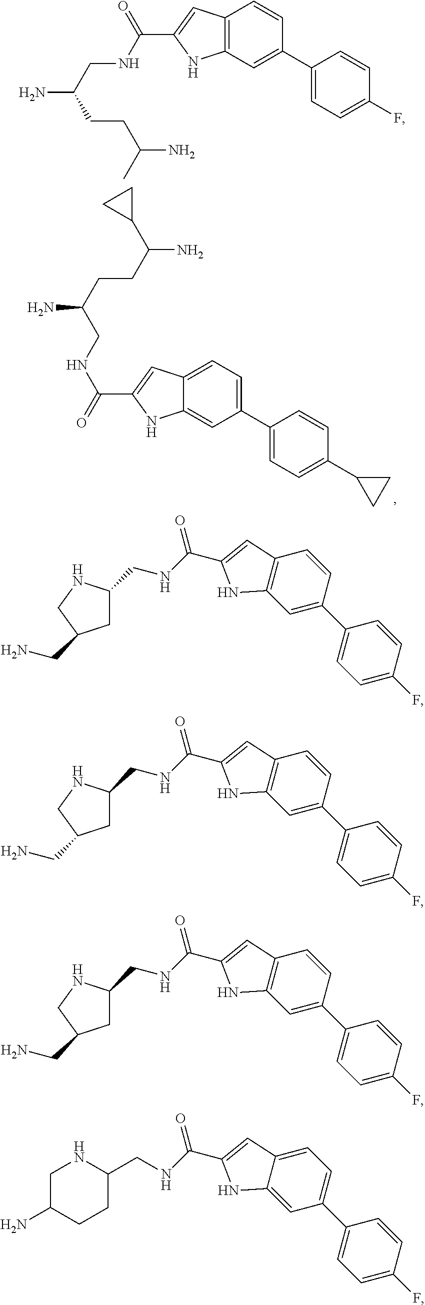 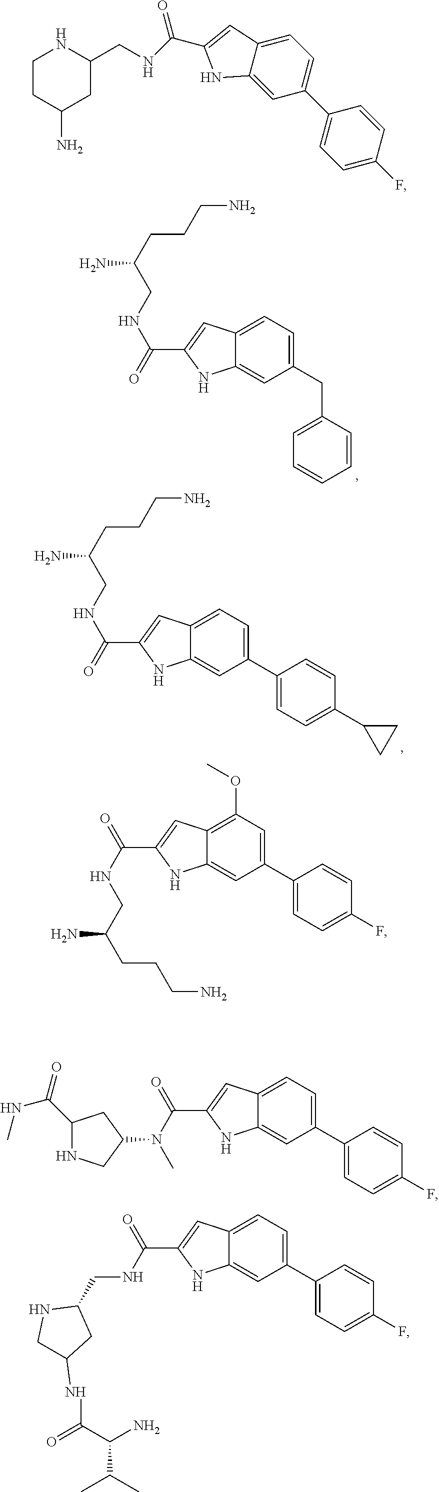 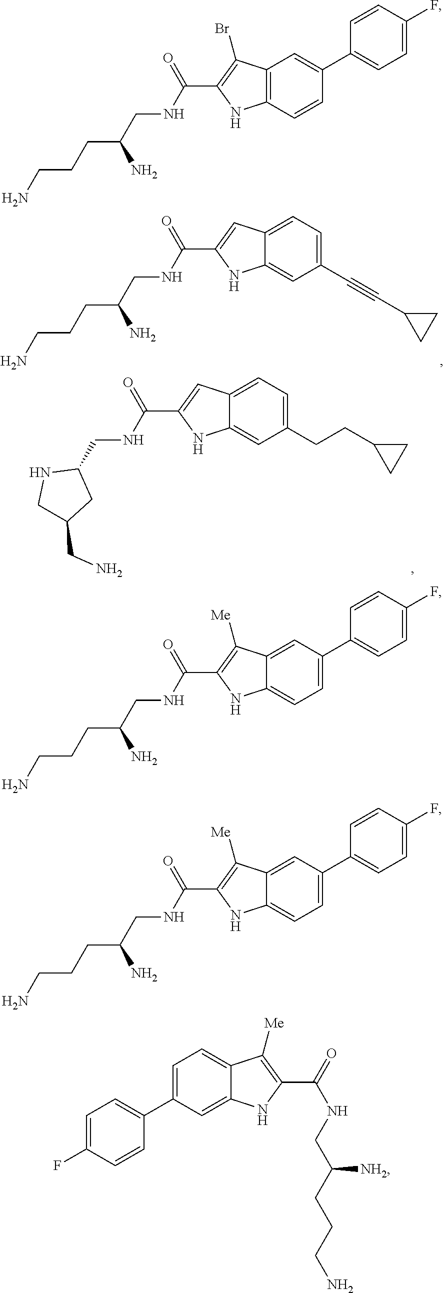 [SMILES: NCCC[C@H](N)CNC(=O)c1[nH]c2ccc(-c3ccc(F)cc3)cc2c1Br ; NCCC[C@H](N)CNC(=O)c1cc2ccc(C#CC3CC3)cc2[nH]1 ; NC[C@H]1CN[C@H](CNC(=O)c2cc3ccc(CCC4CC4)cc3[nH]2)C1 ; Cc1c(C(=O)NC[C@@H](N)CCCN)[nH]c2ccc(-c3ccc(F)cc3)cc12 ; Cc1c(C(=O)NC[C@@H](N)CCCN)[nH]c2ccc(-c3ccc(F)cc3)cc12 ; Cc1c(C(=O)NC[C@@H](N)CCCN)[nH]c2cc(-c3ccc(F)cc3)ccc12] 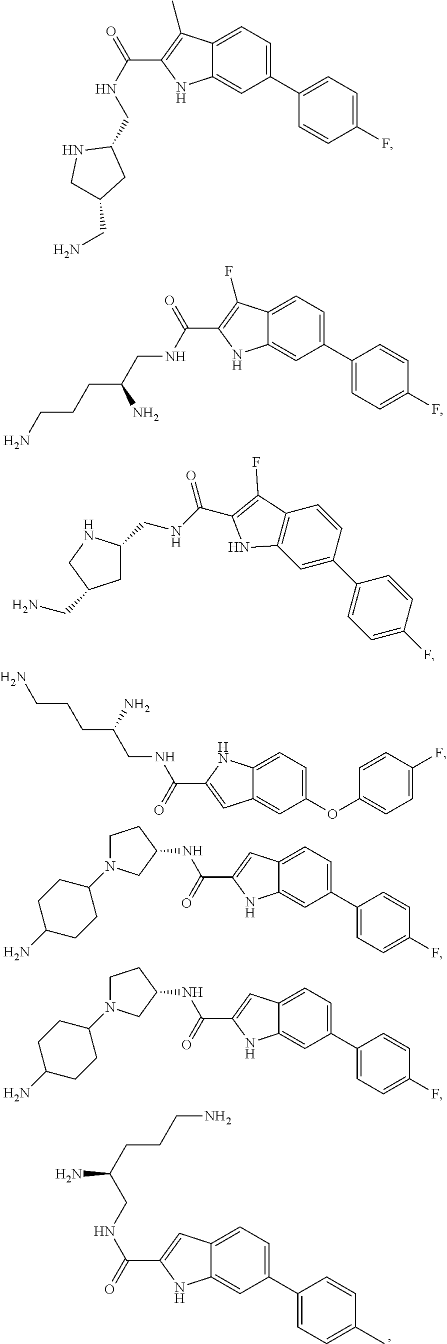 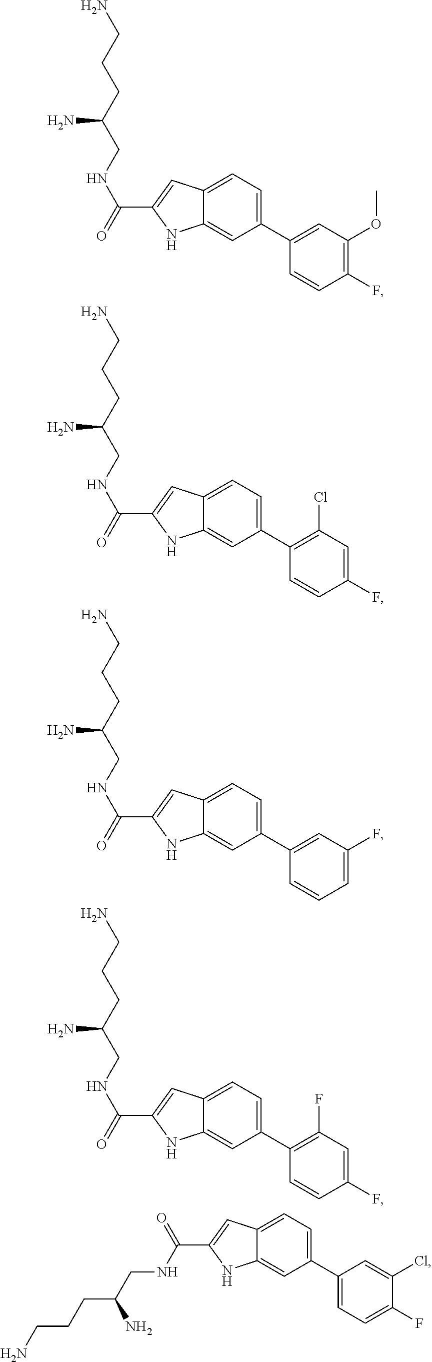 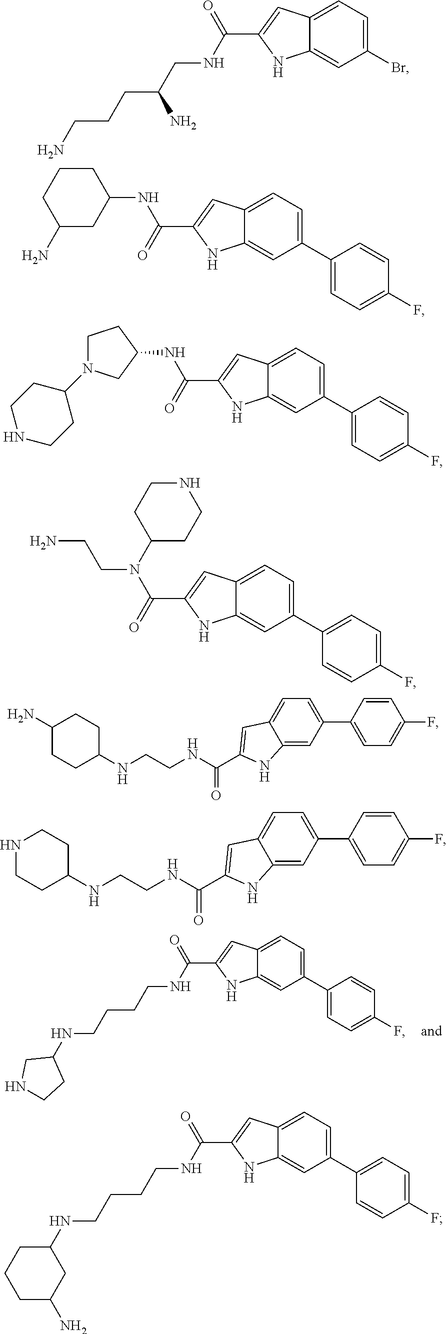
or a pharmaceutically acceptable salt thereof.

9. The method of claim 1 wherein the bacterial efflux pump inhibitor is selected from the group consisting of: 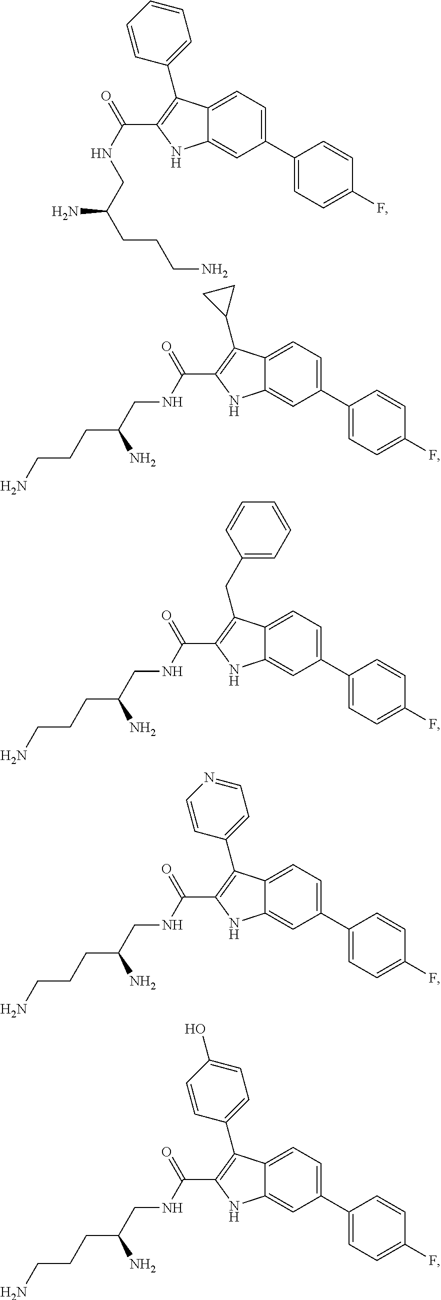 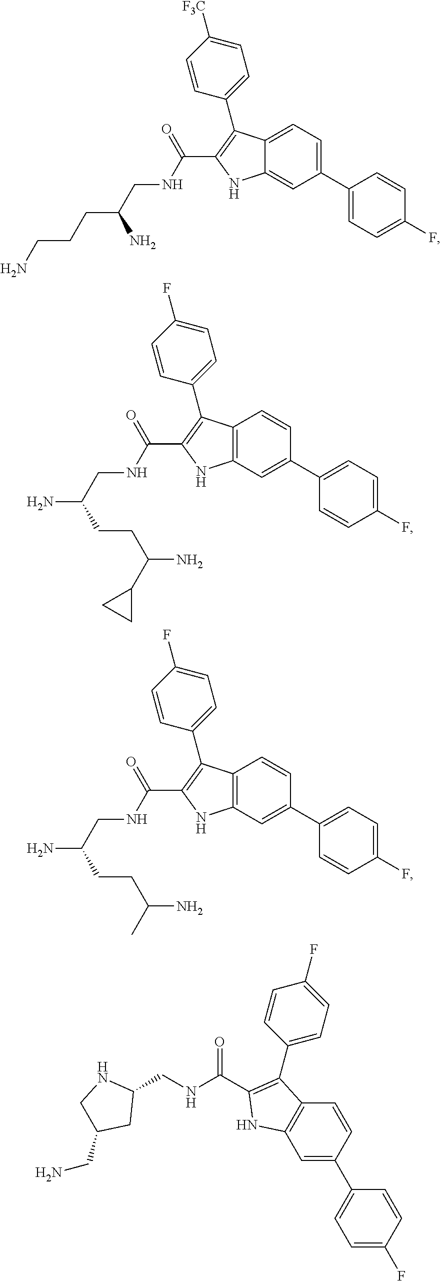 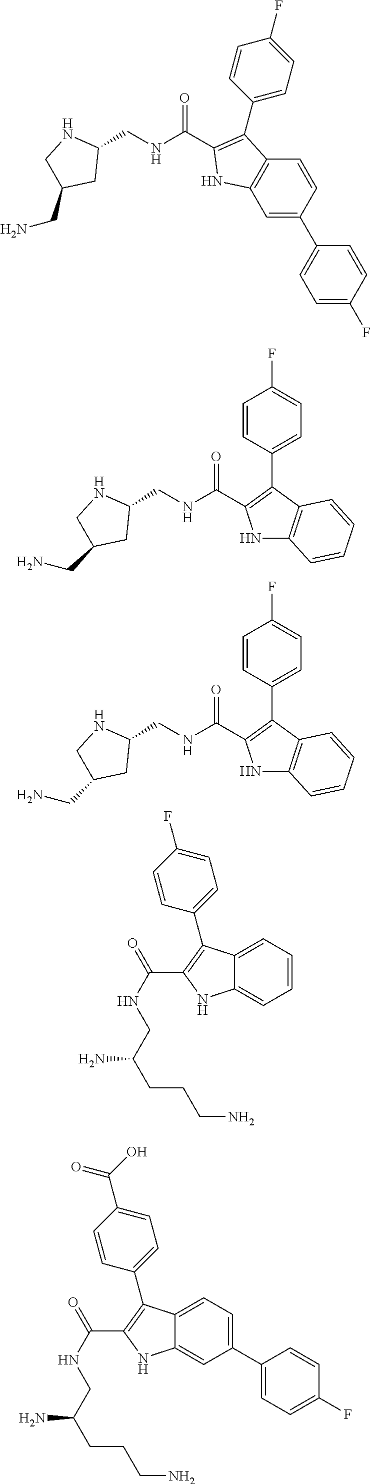 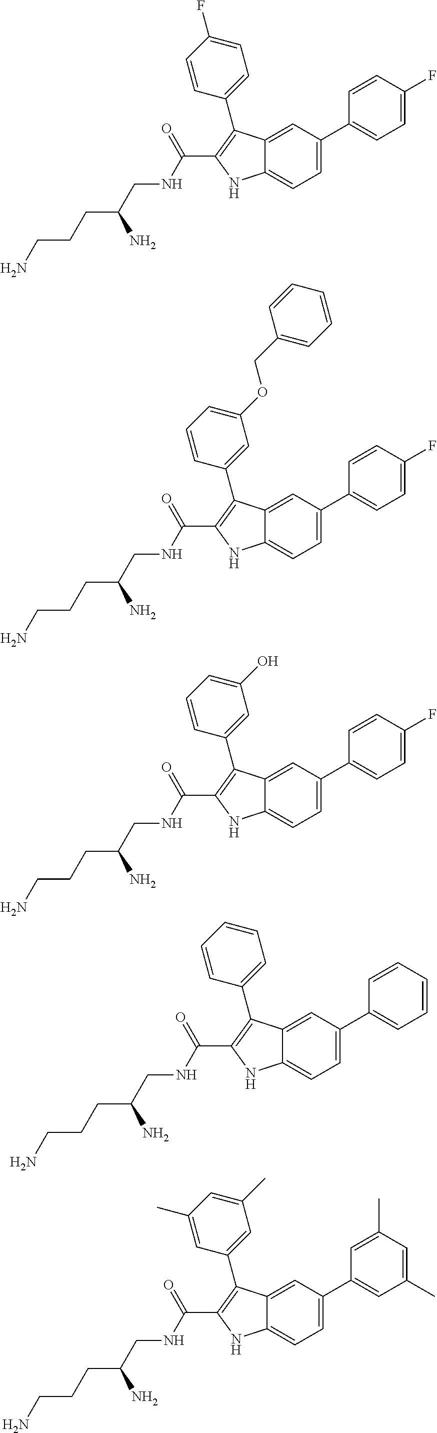 [SMILES: NCCC[C@H](N)CNC(=O)c1[nH]c2ccc(-c3ccc(F)cc3)cc2c1-c1ccc(F)cc1 ; NCCC[C@H](N)CNC(=O)c1[nH]c2ccc(-c3ccc(F)cc3)cc2c1-c1cccc(OCc2ccccc2)c1 ; NCCC[C@H](N)CNC(=O)c1[nH]c2ccc(-c3ccc(F)cc3)cc2c1-c1cccc(O)c1 ; NCCC[C@H](N)CNC(=O)c1[nH]c2ccc(-c3ccccc3)cc2c1-c1ccccc1 ; Cc1cc(C)cc(-c2ccc3[nH]c(C(=O)NC[C@@H](N)CCCN)c(-c4cc(C)cc(C)c4)c3c2)c1] 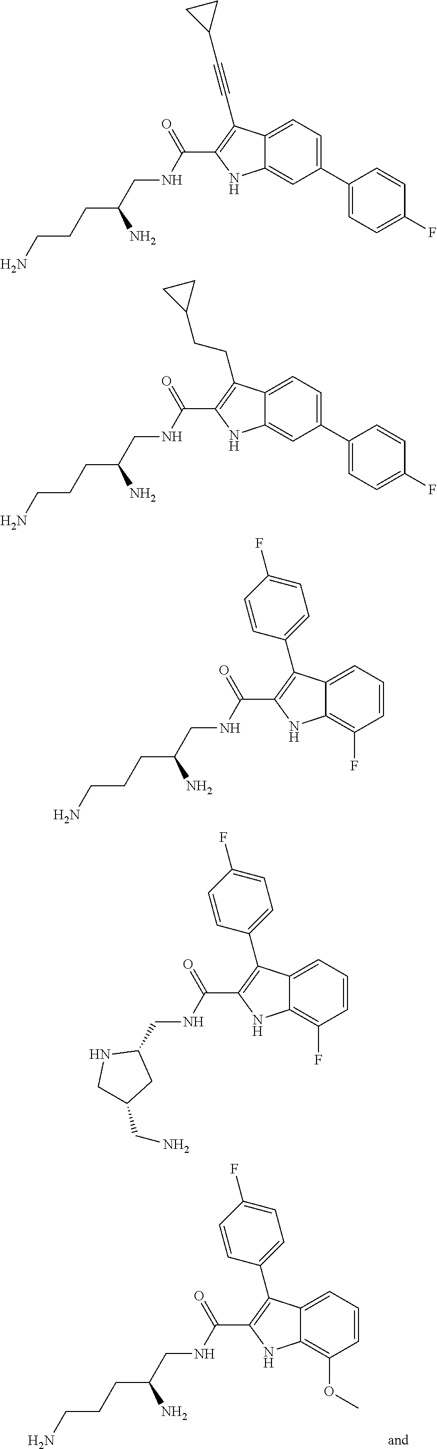 [SMILES: NCCC[C@H](N)CNC(=O)c1[nH]c2cc(-c3ccc(F)cc3)ccc2c1C#CC1CC1 ; NCCC[C@H](N)CNC(=O)c1[nH]c2cc(-c3ccc(F)cc3)ccc2c1CCC1CC1 ; NCCC[C@H](N)CNC(=O)c1[nH]c2c(F)cccc2c1-c1ccc(F)cc1 ; NC[C@@H]1CN[C@H](CNC(=O)c2[nH]c3c(F)cccc3c2-c2ccc(F)cc2)C1 ; COc1cccc2c(-c3ccc(F)cc3)c(C(=O)NC[C@@H](N)CCCN)[nH]c12] 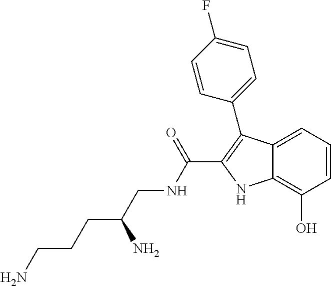 [SMILES: NCCC[C@H](N)CNC(=O)c1[nH]c2c(O)cccc2c1-c1ccc(F)cc1]
or a pharmaceutically acceptable salt thereof.

10. The method of claim 1, wherein the compound of formula I has the following formula Ia: 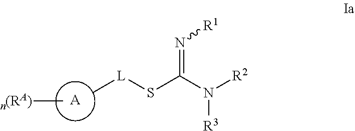
or a pharmaceutically acceptable salt thereof, wherein:
R¹ is hydrogen, (C₁-C₄)alkyl, or (C₁-C₄)haloalkyl;
R² is hydrogen, (C₁-C₄)alkyl, or (C₁-C₄)haloalkyl;
R³ is hydrogen, (C₁-C₄)alkyl, or (C₁-C₄)haloalkyl;
L is (C₁-C₈)alkylene that is optionally substituted with one or more R^{L} groups;
each R^{L} is independently selected from hydrogen, halo, hydroxy, nitro, cyano, or (C₁-C₄)alkoxy; or any two R^{L} groups that are attached to the same carbon taken together form a (C₃-C₆)carbocycle;
A is aryl or heteroaryl;
each R^{A} is independently selected from the group consisting of halo, cyano, nitro, (C₁-C₆)alkyl, (C₁-C₆)alkoxy, —NR^{X}R^{Y}, aryl or heteroaryl; wherein the (C₁-C₆)alkyl and (C₁-C₆)alkoxy is optionally substituted with one or more groups selected from oxo, halo, hydroxy, (C₁-C₄)alkoxy, nitro, cyano, or —NR^{X}R^{Y}; wherein the aryl and heteroaryl is optionally substituted with one or more groups selected from halo, hydroxy, (C₁-C₄)alkyl, (C₁-C₄)haloalkyl, (C₁-C₄)alkoxy, nitro, cyano, or —NR^{X}R^{Y};
each R^{X} and R^{Y} are independently hydrogen or (C₁-C₄)alkyl; or R^{X} and R^{Y} taken together with the nitrogen to which they are attached form pyrrolidinyl, piperidinyl, piperazinyl or morpholinyl; and
n is 0, 1, 2, 3, or 4.

11. The method of claim 1, wherein R¹ is hydrogen.

12. The method of claim 1, wherein R² is hydrogen.

13. The method of claim 1, wherein R³ is hydrogen or methyl.

14. The method of claim 1, wherein the compound of formula I has the following formula Ib: 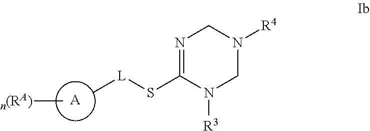
or a pharmaceutically acceptable salt thereof, wherein:
R³ is hydrogen, (C₁-C₄)alkyl or (C₁-C₄)haloalkyl;
R⁴ is hydrogen, (C₁-C₆)alkyl, aryl or heteroaryl, wherein the (C₁-C₆)alkyl is optionally substituted with one or more groups selected from halo, hydroxy, nitro, cyano, (C₁-C₄)alkoxy, —NR^{X}R^{Y}, aryl, heteroaryl, aryloxy, or heteroaryloxy, wherein any ary, heteroaryl, aryloxy and heteroaryloxy is optionally substituted with one or more groups selected from halo, hydroxy, (C₁-C₄)alkyl, (C₁-C₄)haloalkyl, (C₁-C₄)alkoxy, nitro, cyano, or —NR^{X}R^{Y};
L is (C₁-C₈)alkylene that is optionally substituted with one or more R^{L} groups;
each R^{L} is independently selected from hydrogen, halo, hydroxy, nitro, cyano, or (C₁-C₄)alkoxy; or any two R^{L} groups that are attached to the same carbon taken together form a (C₃-C₆)carbocycle;
A is aryl or heteroaryl;
each R^{A} is independently selected from the group consisting of halo, cyano, nitro, (C₁-C₆)alkyl, (C₁-C₆)alkoxy, —NR^{X}R^{Y}, aryl or heteroaryl; wherein the (C₁-C₆)alkyl and (C₁-C₆)alkoxy is optionally substituted with one or more groups selected from oxo, halo, hydroxy, (C₁-C₄)alkoxy, nitro, cyano, or —NR^{X}R^{Y}; wherein the aryl and heteroaryl is optionally substituted with one or more groups selected from halo, hydroxy, (C₁-C₄)alkyl, (C₁-C₄)haloalkyl, (C₁-C₄)alkoxy, nitro, cyano, or —NR^{X}R^{Y};
each R^{X} and R^{Y} are independently hydrogen or (C₁-C₄)alkyl; or R^{X} and R^{Y} taken together with the nitrogen to which they are attached form pyrrolidinyl, piperidinyl, piperazinyl or morpholinyl; and
n is 0, 1, 2, 3, or 4.

15. (canceled)

16. The method of claim 1, wherein R⁴ is (C₁-C₆)alkyl, wherein the (C₁-C₆)alkyl is optionally substituted with one or more groups selected from halo, hydroxy, halo, nitro, cyano, (C₁-C₄)alkoxy, or phenyl, wherein the phenyl is optionally substituted with one or more groups selected from halo, hydroxy, (C₁-C₄)alkyl, (C₁-C₄)haloalkyl, (C₁-C₄)alkoxy, nitro or cyano.

17-18. (canceled)

19. The method of claim 1, wherein A is phenyl, naphthyl, 5-6 membered monocyclic heteroary, or 9-10 membered bicyclic heteroaryl.

20-24. (canceled)

25. The method of claim 1 wherein the compound of formula I is selected from the group consisting of: 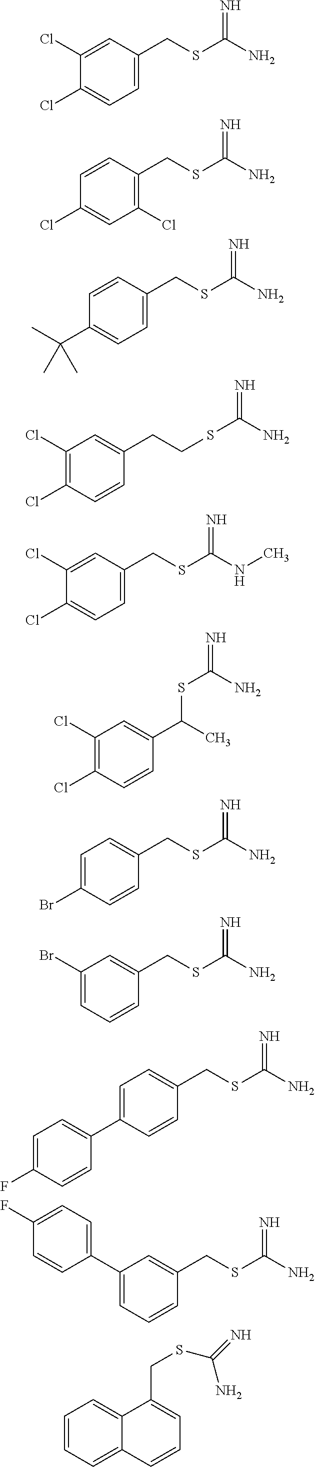 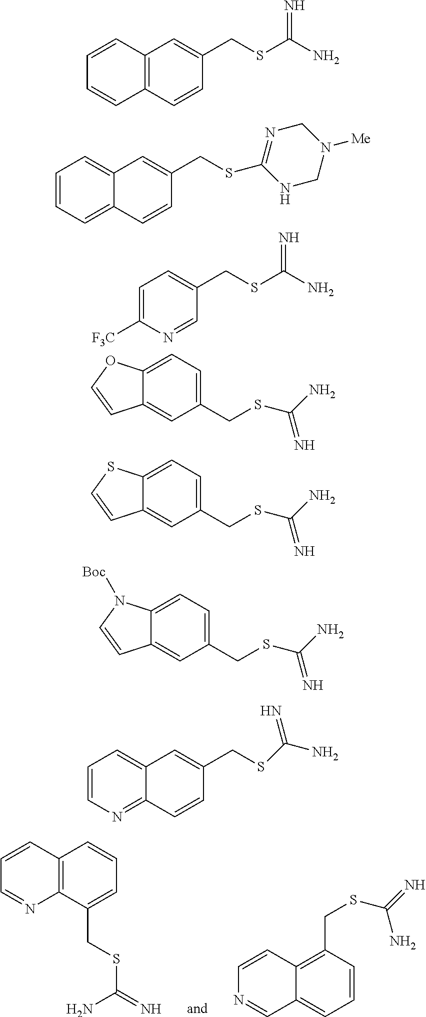
or a pharmaceutically acceptable salt thereof.

26-32. (canceled)

33. A compound of formula Ia or Ib: 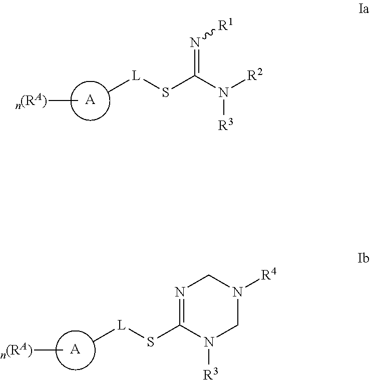
or a salt thereof, wherein:
R¹ is hydrogen, (C₁-C₄)alkyl, or (C₁-C₄)haloalkyl;
R² is hydrogen, (C₁-C₄)alkyl, or (C₁-C₄)haloalkyl;
R³ is hydrogen, (C₁-C₄)alkyl, or (C₁-C₄)haloalkyl;
R⁴ is hydrogen, (C₁-C₆)alkyl, aryl or heteroaryl, wherein the (C₁-C₆)alkyl is optionally substituted with one or more groups selected from halo, hydroxy, nitro, cyano, (C₁-C₄)alkoxy, —NR^{X}R^{Y}, aryl, heteroaryl, aryloxy, or heteroaryloxy, wherein any ary, heteroaryl, aryloxy and heteroaryloxy is optionally substituted with one or more groups selected from halo, hydroxy, (C₁-C₄)alkyl, (C₁-C₄)haloalkyl, (C₁-C₄)alkoxy, nitro, cyano, or —NR^{X}R^{Y};
L is (C₁-C₅)alkylene that is optionally substituted with one or more R^{L} groups;
each R^{L} is independently selected from hydrogen, halo, hydroxy, nitro, cyano, or (C₁-C₄)alkoxy; or any two R^{L} groups that are attached to the same carbon taken together form a (C₃-C₆)carbocycle;
A is aryl or heteroaryl;
each R^{A} is independently selected from the group consisting of halo, cyano, nitro, (C₁-C₆)alkyl, (C₁-C₆)alkoxy, —NR^{X}R^{Y}, aryl or heteroaryl; wherein the (C₁-C₆)alkyl and (C₁-C₆)alkoxy is optionally substituted with one or more groups selected from oxo, halo, hydroxy, (C₁-C₄)alkoxy, nitro, cyano, or —NR^{X}R^{Y}; wherein the aryl and heteroaryl is optionally substituted with one or more groups selected from halo, hydroxy, (C₁-C₄)alkyl, (C₁-C₄)haloalkyl, (C₁-C₄)alkoxy, nitro, cyano, or —NR^{X}R^{Y};
each R^{X} and R^{Y} are independently hydrogen or (C₁-C₄)alkyl; or R^{X} and R^{Y} taken together with the nitrogen to which they are attached form pyrrolidinyl, piperidinyl, piperazinyl, or morpholinyl; and
n is 0, 1, 2, 3, or 4;
provided that when the compound is a compound of formula Ia and A is phenyl, then the A is substituted with at least one phenyl group; and
provided that the compound of formula Ib is not 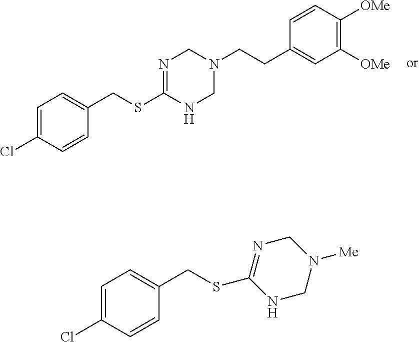 [SMILES: COc1ccc(CCN2CN=C(SCc3ccc(Cl)cc3)NC2)cc1OC ; "or" ; CN1CN=C(SCc2ccc(Cl)cc2)NC1]
or a salt thereof.

34-48. (canceled)

49. The compound of claim 33 which is selected from the group consisting of: 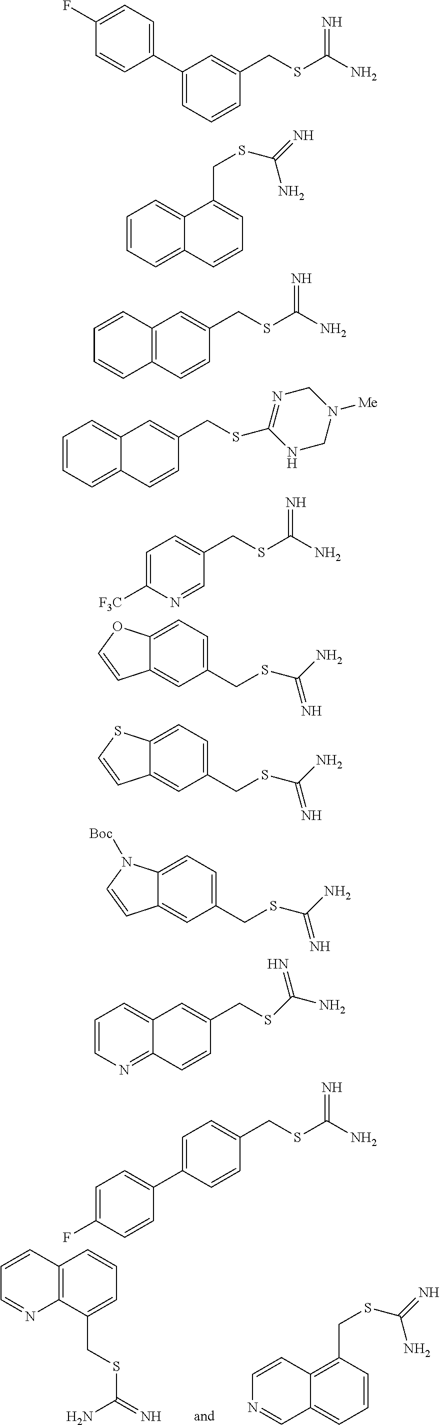
or a salt thereof.

50. A pharmaceutical composition comprising a compound of formula Ia or Ib as described in claim 33, or a pharmaceutically acceptable salt thereof, and a pharmaceutically acceptable vehicle.

51. A method of treating or preventing a bacterial infection in an animal comprising administering to the animal a compound of formula Ia or Ib, or a pharmaceutically acceptable salt thereof, as described in claim 33.

52-57. (canceled)
